SEQUENCE LISTING (106 sequences)

SEQ ID NO: 1 - protein, 8 aa, synthetic construct
LLMWITQC

SEQ ID NO: 2 - protein, 8 aa, synthetic construct
LAMWITQC

SEQ ID NO: 3 - protein, 9 aa, synthetic construct
NVLLWITAL

SEQ ID NO: 4 - protein, 9 aa, synthetic construct
EVDPIGHLY

SEQ ID NO: 5 - protein, 9 aa, synthetic construct
ESDPIVAQY

SEQ ID NO: 6 - protein, 9 aa, synthetic construct
DLLNHIATV

SEQ ID NO: 7 - protein, 9 aa, synthetic construct
SLLLWISGA

SEQ ID NO: 8 - protein, 9 aa, synthetic construct
VLIDWINDV

SEQ ID NO: 9 - protein, 10 aa, synthetic construct
HTWERMWMHV

SEQ ID NO: 10 - protein, 9 aa, synthetic construct
QVGMWVWEA

SEQ ID NO: 11 - protein, 9 aa, synthetic construct
LILSYIAGI

SEQ ID NO: 12 - protein, 10 aa, synthetic construct
EQKLISEEDL

SEQ ID NO: 13 - DNA, 31 bp, synthetic construct
  source 1..31: mol_type = other DNA
ggccgtattggccccgccacctgtgagcggg

SEQ ID NO: 14 - DNA, 26 bp, synthetic construct
  source 1..26: mol_type = other DNA
cgactcactatagggccgtattggcc

SEQ ID NO: 15 - DNA, 23 bp, synthetic construct
  source 1..23: mol_type = other DNA
agtgatttccggcctgtttggcc

SEQ ID NO: 16 - DNA, 46 bp, synthetic construct
  source 1..46: mol_type = other DNA
gacctccctcacgctgtttctagcactcttggccgtattggccccg

SEQ ID NO: 17 - DNA, 46 bp, synthetic construct
  source 1..46: mol_type = other DNA
ctttccctacacgacgctcttccgatctttggcctgtttggcctta

SEQ ID NO: 18 - DNA, 25 bp, synthetic construct
  source 1..25: mol_type = other DNA
aatgatacggcgaccaccgagatct

SEQ ID NO: 19 - DNA, 33 bp, synthetic construct
  source 1..33: mol_type = other DNA
gtgactggagttcagacgtgtgctcttccgatc

SEQ ID NO: 20 - DNA, 16 bp, synthetic construct
  source 1..16: mol_type = other DNA
gccacctgtgagcggg

SEQ ID NO: 21 - DNA, 21 bp, synthetic construct
  source 1..21: mol_type = other DNA
tctttggcctgtttggcctta

SEQ ID NO: 22 - RNA, 20 nt, synthetic construct
  source 1..20: mol_type = other RNA
cgccaggccgagccagttcc

SEQ ID NO: 23 - RNA, 20 nt, synthetic construct
  source 1..20: mol_type = other RNA
ggcgacacccggaactggct

SEQ ID NO: 24 - RNA, 20 nt, synthetic construct
  source 1..20: mol_type = other RNA
cacccggaactggctcggcc

SEQ ID NO: 25 - RNA, 20 nt, synthetic construct
  source 1..20: mol_type = other RNA
ccggaactggctcggcctgg

SEQ ID NO: 26 - protein, 9 aa, synthetic construct
AAGIGILTV

SEQ ID NO: 27 - protein, 9 aa, synthetic construct
ELSDWIHQL

SEQ ID NO: 28 - protein, 9 aa, synthetic construct
LVMQWLGQI

SEQ ID NO: 29 - protein, 9 aa, synthetic construct
VVNPWLTQV

SEQ ID NO: 30 - protein, 9 aa, synthetic construct
TQIQWATQV

SEQ ID NO: 31 - protein, 9 aa, synthetic construct
TLLLWLCQA

SEQ ID NO: 32 - protein, 9 aa, synthetic construct
SQCMWLMQA

SEQ ID NO: 33 - DNA, 708 bp, synthetic construct
  source 1..708: mol_type = other DNA
atggccttaccagtgaccgccttgctcctgccgctggccttgctgctccacgccgccaggccgagccagttccgggtgtcgccgctggatcggacctggaacctgggcgagacagtggagctgaagtgccaggtgctgctgtccaacccgacgtcgggctgctcgtggctcttccagccgcgcggcgccgccgccagtcccaccttcctcctatacctctcccaaaacaagcccaaggcggccgaggggctggacacccagcggttctcgggcaagaggttgggggacaccttcgtcctcaccctgagcgacttccgccgagagaacgagggctactatttctgctcggccctgagcaactccatcatgtacttcagccacttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaaccgaagacgtgtttgcaaatgtccccggcctgtggtcaaatcgggagacaagcccagcctttcggcgagatacgtctaa

SEQ ID NO: 34 - DNA, 708 bp, synthetic construct
  source 1..708: mol_type = other DNA
atggccttaccagtgaccgccttgctcctgccgctggccttgctgctccacgccgccaggccgagccagttccgggtgtcgccgctggatcggacctggaacctgggcgagacagtggagctgaagtgccaggtgctgggttccaacccgacgtcgggctgctcgtggctcttccagccgcgcggcgccgccgccagtcccaccttcctcctatacctctcccaaaacaagcccaaggcggccgaggggctggacacccagcggttctcgggcaagaggttgggggacaccttcgtcctcaccctgagcgacttccgccgagagaacgagggctactatttctgctcggccctgagcaactccatcatgtacttcagccacttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaaccgaagacgtgtttgcaaatgtccccggcctgtggtcaaatcgggagacaagcccagcctttcggcgagatacgtctaa

SEQ ID NO: 35 - DNA, 708 bp, synthetic construct
  source 1..708: mol_type = other DNA
atggccttaccagtgaccgccttgctcctgccgctggccttgctgctccacgccgccaggccgagccagttccgggtgtcgccgctggatcggacctggaacctgggcgagacagtggagctgaagtgccaggtgctgctgtccaacccgacgtcgggctgctcgtggctcttccagccgcgcggcgccgccgccagtcccaccttcctcctatacctctcccataacaagcccactgcggccgaggggctggacacccagcggttctcgggcaagaggttgggggacaccttcgtcctcaccctgagcgacttccgccgagagaacgagggctactatttctgctcggccctgagcaactccatcatgtacttcagccacttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaaccgaagacgtgtttgcaaatgtccccggcctgtggtcaaatcgggagacaagcccagcctttcggcgagatacgtctaa

SEQ ID NO: 36 - protein, 235 aa, synthetic construct
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 37 - protein, 235 aa, synthetic construct
MALPVTALLLPLALLLHAARPSQFRVSPLDRTWNLGETVELKCQVLGSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 38 - protein, 235 aa, synthetic construct
MALPVTALLLPLALLLHAARPSQFRVSPLDRTWNLGETVELKCQVLLSNPTSGCSWLFQPRGAAASPTFLLYLSHNKPTAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 39 - protein, 9 aa, synthetic construct
SAFEFLSSA

SEQ ID NO: 40 - protein, 9 aa, synthetic construct
SLLMWITQC

SEQ ID NO: 41 - RNA, 20 nt, synthetic construct
  source 1..20: mol_type = other RNA
tcagtaacatgcgcatctac

SEQ ID NO: 42 - RNA, 20 nt, synthetic construct
  source 1..20: mol_type = other RNA
ggcgcgccacgatgcggccg

SEQ ID NO: 43 - protein, 10 aa, synthetic construct
SMAGIGIVDV

SEQ ID NO: 44 - protein, 10 aa, synthetic construct
NLSNLGILPV

SEQ ID NO: 45 - protein, 10 aa, synthetic construct
SMLGIGIVPV

SEQ ID NO: 46 - protein, 10 aa, synthetic construct
IMEDVGWLNV

SEQ ID NO: 47 - protein, 214 aa, synthetic construct
SQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 48 - protein, 214 aa, synthetic construct
SQFRVSPLDRTWNLGETVELKCQVLGSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 49 - protein, 214 aa, synthetic construct
SQFRVSPLDRTWNLGETVELKCQVLLSNPTSGCSWLFQPRGAAASPTFLLYLSHNKPTAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 50 - protein, 6 aa, synthetic construct
HHHHHH

SEQ ID NO: 51 - DNA, 38 bp, synthetic construct
  source 1..38: mol_type = other DNA
cagaggctagaattctgcagacgttactggccgaagcc

SEQ ID NO: 52 - DNA, 39 bp, synthetic construct
  source 1..39: mol_type = other DNA
catggtggctattatcatcgtgtttttcaaaggaaaacc

SEQ ID NO: 53 - DNA, 24 bp, synthetic construct
  source 1..24: mol_type = other DNA
ccaggtgctgggttccaacccgac

SEQ ID NO: 54 - DNA, 18 bp, synthetic construct
  source 1..18: mol_type = other DNA
cacttcagctccactgtc

SEQ ID NO: 55 - DNA, 25 bp, synthetic construct
  source 1..25: mol_type = other DNA
gcccactgcggccgaggggctggac

SEQ ID NO: 56 - DNA, 36 bp, synthetic construct
  source 1..36: mol_type = other DNA
ttgttatgggagaggtataggaggaaggtgggactg

SEQ ID NO: 57 - DNA, 29 bp, synthetic construct
  source 1..29: mol_type = other DNA
gatgataatagccaccatggccttaccag

SEQ ID NO: 58 - DNA, 42 bp, synthetic construct
  source 1..42: mol_type = other DNA
atcccgggcccgcggtaccgttagacgtatctcgccgaaagg

SEQ ID NO: 59 - protein, 8 aa, synthetic construct
SLLWISGA

SEQ ID NO: 60 - protein, 9 aa, synthetic construct
ALLMLVTLT

SEQ ID NO: 61 - protein, 9 aa, synthetic construct
TLLMVLTRI

SEQ ID NO: 62 - protein, 9 aa, synthetic construct
VLLCWIKFL

SEQ ID NO: 63 - protein, 9 aa, synthetic construct
SLLICVAAV

SEQ ID NO: 64 - protein, 9 aa, synthetic construct
AMDLIISTL

SEQ ID NO: 65 - protein, 9 aa, synthetic construct
GILMVIDPL

SEQ ID NO: 66 - protein, 9 aa, synthetic construct
SVIMELLSV

SEQ ID NO: 67 - protein, 9 aa, synthetic construct
VVPNWLTQV

SEQ ID NO: 68 - protein, 9 aa, synthetic construct
ELDSWIHQL

SEQ ID NO: 69 - protein, 8 aa, synthetic construct
VNPWLTQV

SEQ ID NO: 70 - protein, 9 aa, synthetic construct
VMLAWLTGT

SEQ ID NO: 71 - protein, 8 aa, synthetic construct
VVNPWTQV

SEQ ID NO: 72 - protein, 10 aa, synthetic construct
FIAAMWGIFM

SEQ ID NO: 73 - protein, 9 aa, synthetic construct
MLAAALDAV

SEQ ID NO: 74 - protein, 9 aa, synthetic construct
SVITYVSSI

SEQ ID NO: 75 - protein, 9 aa, synthetic construct
MVPLWISSV

SEQ ID NO: 76 - protein, 9 aa, synthetic construct
FLHAAALAV

SEQ ID NO: 77 - protein, 8 aa, synthetic construct
ALAAALYV

SEQ ID NO: 78 - protein, 9 aa, synthetic construct
ALAAVALMV

SEQ ID NO: 79 - protein, 9 aa, synthetic construct
NLAAGLAAV

SEQ ID NO: 80 - protein, 9 aa, synthetic construct
OLAAAHLAV

SEQ ID NO: 81 - protein, 9 aa, synthetic construct
ALANAFLAV

SEQ ID NO: 82 - protein, 9 aa, synthetic construct
SITMWFSLA

SEQ ID NO: 83 - protein, 8 aa, synthetic construct
MILLIPQV

SEQ ID NO: 84 - protein, 10 aa, synthetic construct
ALAAETALAV

SEQ ID NO: 85 - protein, 9 aa, synthetic construct
ALAAGLAHV

SEQ ID NO: 86 - protein, 10 aa, synthetic construct
ALAEAWALAV

SEQ ID NO: 87 - protein, 10 aa, synthetic construct
ALAAANQLAV

SEQ ID NO: 88 - protein, 8 aa, synthetic construct
AVMWVWEA

SEQ ID NO: 89 - protein, 8 aa, synthetic construct
LLWMITQC

SEQ ID NO: 90 - protein, 9 aa, synthetic construct
TMIFMILGA

SEQ ID NO: 91 - protein, 9 aa, synthetic construct
RTLTYLSQV

SEQ ID NO: 92 - protein, 8 aa, synthetic construct
LLLMVSQL

SEQ ID NO: 93 - protein, 9 aa, synthetic construct
PLLQCITSV

SEQ ID NO: 94 - protein, 9 aa, synthetic construct
SLLDFIRPL

SEQ ID NO: 95 - protein, 10 aa, synthetic construct
SVFHFMWVAV

SEQ ID NO: 96 - protein, 9 aa, synthetic construct
SALCWLLCV

SEQ ID NO: 97 - protein, 9 aa, synthetic construct
SLIKGIARA

SEQ ID NO: 98 - protein, 9 aa, synthetic construct
QLLIWIPRL

SEQ ID NO: 99 - protein, 9 aa, synthetic construct
RLLDWFRSL

SEQ ID NO: 100 - protein, 4 aa, synthetic construct
TQIQ

SEQ ID NO: 101 - protein, 10 aa, synthetic construct
MMWDRGLGMM

SEQ ID NO: 102 - protein, 10 aa, synthetic construct
LMFDRGMSLL

SEQ ID NO: 103 - protein, 10 aa, synthetic construct
NMGGLGIMPV

SEQ ID NO: 104 - protein, 10 aa, synthetic construct
MMWDRGMGLL

SEQ ID NO: 105 - protein, 10 aa, synthetic construct
ILEDRGMGLL

SEQ ID NO: 106 - DNA, 16 bp, synthetic construct
  source 1..16: mol_type = other DNA
taaggccaaacaggcc

(19) United States
(12) Patent Application Publication
(10) Pub. No.: US 2025/0302956 A1
(43) Pub. Date: Oct. 2, 2025

(54) COMPOSITIONS AND METHODS FOR MODULATING TCR SPECIFICITY

(71) Applicants: MEMORIAL SLOAN-KETTERING CANCER CENTER, New York, NY (US); MEMORIAL HOSPITAL FOR CANCER AND ALLIED DISEASES, New York, NY (US); SLOAN-KETTERING INSTITUTE FOR CANCER AND ALLIED DISEASES, New York, NY (US)

(72) Inventors: David A. Scheinberg, New York, NY (US); Heather Jones, New York, NY (US); Zita Aretz, New York, NY (US)

(21) Appl. No.: 18/865,320

(22) PCT Filed: May 11, 2023
(86) PCT No.: PCT/US2023/021871

§ 371 Date: Nov. 12, 2024

Related U.S. Application Data
(60) Provisional application No. 63/341,684, filed on May 13, 2022.

(51) Int. Cl. A61K 40/11; A61K 40/32; A61K 40/42; C07K 14/705; C07K 14/725; C12N 5/0783

(52) U.S. Cl. CPC A61K 40/11; A61K 40/32; A61K 40/42; C07K 14/7051; C07K 14/70517; C12N 5/0638; C12N 2510/00

(57) ABSTRACT

The present technology provides compositions and methods for modulating T Cell Receptor (TCR) specificity as well as methods for treating cancer in a subject in need thereof. The present disclosure provides engineered cytotoxic T cells comprising a TCR and/or nucleic acid encoding the TCR and a mutant CD8 alpha polypeptide and/or nucleic acid encoding the mutant CD8 alpha polypeptide.

### CROSS-REFERENCE TO RELATED APPLICATIONS

This application is the U.S. National Stage Application of International Application No. PCT/US2023/021871, filed May 11, 2023, which claims the benefit of and priority to U.S. Provisional Patent Application No. 63/341,684, filed May 13, 2022, the contents of each of which are incorporated herein by reference in its entirety.

### STATEMENT OF GOVERNMENT INTEREST

This invention was made with government support under CA055349 and CA241894, awarded by the National Institutes of Health. The government has certain rights in the invention.

### SEQUENCE LISTING

The instant application contains a Sequence Listing which has been submitted electronically in XML file format and is hereby incorporated by reference in its entirety. Said XML copy, created on Nov. 11, 2024, is named 115872-2738_SL.xml and is 98,304 bytes in size.

### TECHNICAL FIELD

The present technology relates generally to compositions and methods for modulating T Cell Receptor (TCR) specificity as well as methods for treating cancer in a subject in need thereof. The present disclosure provides engineered cytotoxic T cells comprising a TCR and/or nucleic acid encoding the TCR and a mutant CD8 alpha polypeptide and/or nucleic acid encoding the mutant CD8 alpha polypeptide.

### BACKGROUND

The following description of the background of the present technology is provided simply as an aid in understanding the present technology and is not admitted to describe or constitute prior art to the present technology.

Adoptive cell therapies, in which T cells are exogenously engineered to express a chimeric antigen receptors (CAR), T cell receptors (TCR), or TCR-mimic (TCRm) single chain fragment variable (scFv), that redirect them to tumor associated antigens have been effective as cancer treatments. TCRs and TCRms recognize peptides presented on the cell surface in the context of major histocompatibility complexes (MHC). These peptides are derived from proteins from any cellular location and are therefore not limited to dysregulated, overexpressed, or lineage-specific proteins found on the cell surface, as is the case with CAR T cells and traditional antibodies, thus vastly expanding the repertoire of potentially targetable cancer antigens.

TCRs recognize amino acids as short linear peptides, typically 8-12 amino acids long for MHC class I (MHCI) restricted TCRs, buried in the groove of an MHC protein. Hence, cross-reactivities with presented peptides that have similar amino acid sequences are frequently observed. A major drawback of TCR-based therapies is that it is extremely challenging to predict off-target reactivities that can lead to toxicities or to modulate these detrimental cross reactions. This problem was made vividly evident by severe toxicities associated with some TCR therapies, for example the MAGE-A3 TCR T cells, which resulted in fatal cardiotoxicity due to unpredicted cross reactivity with a peptide derived from cardiac muscle titin protein.

Accordingly, there remains an urgent need for efficient methods to predict and prevent off-target reactivities and potential toxicities of highly promiscuous TCR-based agents. In addition, methods to mitigate the cross-reactions do not currently exist, even if identified.

### SUMMARY OF THE PRESENT TECHNOLOGY

In one aspect, the present disclosure provides an engineered cytotoxic T cell that comprises (a) a T cell receptor (TCR) that binds to a target antigen and/or a nucleic acid encoding the T cell receptor; (b) lacks detectable expression or activity of a wild-type CD8 alpha polypeptide comprising the amino acid sequence of SEQ ID NO: 36 or SEQ ID NO: 47; and (c) comprises a non-endogenous expression vector that includes a nucleic acid sequence encoding a mutant CD8 alpha polypeptide comprising the amino acid sequence of SEQ ID NO: 37, SEQ ID NO: 38, SEQ ID NO: 48, or SEQ ID NO: 49, optionally wherein the nucleic acid sequence of the mutant CD8 alpha polypeptide includes SEQ ID NO: 34 or SEQ ID NO: 35 and/or is operably linked to an expression control sequence. Examples of expression control sequences include, but are not limited to, inducible promoters, constitutive promoters, native promoters, or heterologous promoters.

In some embodiments of the engineered cytotoxic T cell of the present disclosure, the TCR is a native TCR, a non-native TCR, or a mimic TCR. Examples of mimic TCRs include but are not limited to, TCRm Abs, TCRm BITEs, TCRm CARs, and ImmTACs. Additionally or alternatively, in some embodiments, the TCR is IG4, DMF5, or A6. In certain embodiments, the engineered cytotoxic T cell of the present disclosure is derived from an autologous donor or an allogeneic donor.

Additionally or alternatively, in some embodiments of the engineered cytotoxic T cell disclosed herein, the mutant CD8 alpha polypeptide exhibits reduced binding to major histocompatibility complex (MHC) relative to the wild-type CD8 alpha polypeptide.

Additionally or alternatively, in some embodiments, the engineered cytotoxic T cell comprises a deletion, an inversion, a missense mutation, a nonsense mutation, or a frameshift mutation in a nucleic acid sequence encoding the wild-type CD8 alpha polypeptide, optionally wherein the nucleic acid sequence encoding the wild-type CD8 alpha polypeptide is SEQ ID NO: 33. In certain embodiments, the engineered cytotoxic T cell comprises an inhibitory nucleic acid that specifically targets and inhibits the expression of a nucleic acid sequence encoding the wild-type CD8 alpha polypeptide, optionally wherein the nucleic acid sequence encoding the wild-type CD8 alpha polypeptide is SEQ ID NO: 33. In some embodiments, the inhibitory nucleic acid is an antisense oligonucleotide, a siRNA, a sgRNA or a shRNA.

In another aspect, the present disclosure provides an engineered CD4+ helper T cell that comprises (a) a T cell receptor that binds to a target antigen and/or a nucleic acid encoding the T cell receptor; and (b) comprises a non-endogenous expression vector that includes a nucleic acid sequence encoding a CD8 alpha polypeptide comprising the amino acid sequence of SEQ ID NO: 36, SEQ ID NO: 37, SEQ ID NO: 38, SEQ ID NO: 47, SEQ ID NO: 48, or SEQ ID NO: 49, optionally wherein the nucleic acid sequence of the CD8 alpha polypeptide includes SEQ ID NO: 33, SEQ ID NO: 34 or SEQ ID NO: 35 and/or is operably linked to an expression control sequence. Examples of expression control sequences include, but are not limited to, inducible promoters, constitutive promoters, native promoters, or heterologous promoters.

In some embodiments of the engineered CD4+ helper T cell disclosed herein, the TCR is a native TCR, a non-native TCR, or a mimic TCR. Examples of mimic TCRs include but are not limited to, TCRm Abs, TCRm BITEs, TCRm CARs, and ImmTACs. In certain embodiments, the TCR is IG4, DMF5, or A6. In certain embodiments, the engineered CD4+ helper T cell is derived from an autologous donor or an allogeneic donor.

Additionally or alternatively, in some embodiments of the engineered cytotoxic T cell or the engineered CD4+ helper T cell described herein, the non-endogenous expression vector is a plasmid, a cosmid, a bacmid, a bacterial artificial chromosome (BAC), a yeast artificial chromosome (YAC), a viral vector, or a retroviral vector.

In any and all embodiments of the engineered cytotoxic T cell or the engineered CD4+ helper T cell disclosed herein, the target antigen comprises a tumor antigen. Examples of suitable tumor antigens include, but are not limited to, Tyrosinase, NY-ESO-1, CD277-mediated presentation, MAGE-A4, WT1, MAGE-A10, PRAME, EBV LMP2, MAGE-A1, HA-1, HERV-E, CMV pp65, HBV, TRAIL-DR4, HIV SL9, and AFP.

In one aspect, the present disclosure provides a composition comprising an effective amount of any and all embodiments of the engineered cytotoxic T cell or the engineered CD4+ helper T cell described herein, and a pharmaceutically acceptable carrier.

In another aspect, the present disclosure provides a kit comprising an expression vector that includes a nucleic acid sequence encoding a CD8 alpha amino acid sequence of one or more of SEQ ID NO: 36, SEQ ID NO: 37, SEQ ID NO: 38, SEQ ID NO: 47, SEQ ID NO: 48, or SEQ ID NO: 49, optionally wherein the nucleic acid sequence is any one of SEQ ID NO: 33, SEQ ID NO: 34, or SEQ ID NO: 35, and instructions for transducing CD4+ helper T cells with the expression vector. The kit may further comprise a vector encoding an engineered T-cell receptor (TCR) that binds to a target antigen.

In yet another aspect, the present disclosure provides a kit comprising an expression vector that includes a nucleic acid sequence encoding a mutant CD8 alpha amino acid sequence of one or more of SEQ ID NO: 37, SEQ ID NO: 38, SEQ ID NO: 48, or SEQ ID NO: 49, optionally wherein the nucleic acid sequence is SEQ ID NO: 34 or SEQ ID NO: 35, and instructions for transducing cytotoxic T cells with the expression vector. The kit may further comprise a vector encoding an engineered T-cell receptor (TCR) that binds to a target antigen.

Also disclosed herein are methods for treating cancer or inhibiting tumor growth in a subject in need thereof comprising administering to the subject an effective amount of any and all embodiments of the engineered cytotoxic T cell or the engineered CD4+ helper T cell described herein, or a composition comprising an effective amount of any and all embodiments of the engineered cytotoxic T cell or the engineered CD4+ helper T cell described herein, and a pharmaceutically acceptable carrier.

In one aspect, the present disclosure provides a method for mitigating off-target reactivity/toxicity in a subject receiving adoptive T cell therapy comprising administering to the subject an effective amount of any and all embodiments of the engineered cytotoxic T cell or the engineered CD4+ helper T cell described herein, or a composition comprising an effective amount of any and all embodiments of the engineered cytotoxic T cell or the engineered CD4+ helper T cell described herein, and a pharmaceutically acceptable carrier.

In any of the preceding embodiments of the methods disclosed herein, the subject suffers from or is diagnosed with cancer. In certain embodiments, the cancer or tumor is selected from the group consisting of adrenal cancers, bladder cancers, blood cancers, bone cancers, brain cancers, breast cancers, carcinoma, cervical cancers, colon cancers, colorectal cancers, corpus uterine cancers, ear, nose and throat (ENT) cancers, endometrial cancers, esophageal cancers, gastrointestinal cancers, head and neck cancers, Hodgkin's disease, intestinal cancers, kidney cancers, larynx cancers, acute and chronic leukemias, liver cancers, lymph node cancers, lymphomas, lung cancers, melanomas, mesothelioma, myelomas, nasopharynx cancers, neuroblastomas, non-Hodgkin's lymphoma, oral cancers, ovarian cancers, pancreatic cancers, penile cancers, pharynx cancers, prostate cancers, rectal cancers, sarcoma, seminomas, skin cancers, stomach cancers, teratomas, testicular cancers, thyroid cancers, uterine cancers, vaginal cancers, vascular tumors, and metastases thereof.

Additionally or alternatively, in some embodiments of the methods disclosed herein, the engineered cytotoxic T cell or engineered CD4+ helper T cell is administered pleurally, intravenously, subcutaneously, intranodally, intratumorally, intrathecally, intrapleurally or intraperitoneally.

In another aspect, the present disclosure provides, among other things, a method of preparing cytotoxic T cells for adoptive cell therapy comprising: isolating cytotoxic T cells from a donor subject; inactivating expression and/or activity of a wild-type CD8 alpha polypeptide comprising the amino acid sequence of SEQ ID NO: 36 or SEQ ID NO: 47 in the cytotoxic T cells; transducing the cytotoxic T cells with a non-endogenous expression vector that includes a nucleic acid sequence encoding a mutant CD8 alpha polypeptide comprising the amino acid sequence of SEQ ID NO: 37, SEQ ID NO: 38, SEQ ID NO: 48, or SEQ ID NO: 49, optionally wherein the nucleic acid sequence of the mutant CD8 alpha polypeptide includes SEQ ID NO: 34 or SEQ ID NO: 35; and administering the transduced cytotoxic T cells to a recipient subject. In yet another aspect, the present disclosure provides a method of preparing CD4+ helper T cells for adoptive cell therapy comprising: isolating CD4+ helper T cells from a donor subject; transducing the CD4+ helper T cells with a non-endogenous expression vector that includes a nucleic acid sequence encoding a CD8 alpha polypeptide comprising the amino acid sequence of SEQ ID NO: 36, SEQ ID NO: 37, SEQ ID NO: 38, SEQ ID NO: 47, SEQ ID NO: 48, or SEQ ID NO: 49, optionally wherein the nucleic acid sequence of the CD8 alpha polypeptide includes SEQ ID NO: 33, SEQ ID NO: 34 or SEQ ID NO: 35; and administering the transduced CD4+ helper T cells to a recipient subject. In some embodiments, a donor subject and a recipient subject are the same or different. In some embodiments of the methods disclosed herein, the transduced cytotoxic T cells or the transduced CD4+ helper T cells comprise a native T cell receptor (TCR), a non-native TCR, or a mimic TCR. Examples of mimic TCRs include but are not limited to, TCRm Abs, TCRm BITEs, TCRm CARs, and ImmTACs.

### BRIEF DESCRIPTION OF THE DRAWINGS

FIGS. 1A-1D show PresentER coculture cytotoxicity assays identify off targets of the 1G4 TCR. FIG. 1A shows a PresentER plasmid design. Minigene antigens are encoded down stream of an ER signaling sequence. Genes encoding GFP and puromycin resistance are separated by an IRES site. FIG. 1B demonstrates specific lysis of NY-ESO-1 or MART-1 expressing T2 cells by 1G4 TCR (2) or UNTD (1) T cells from three different HLA-A*02:01 negative donors after 48 hours of coculture. Target killing was detected by flow cytometry. FIG. 1C shows a method of coculture library screening: T2 cells expressing a library of peptides (multicolored) are cocultured with either TCR (black; 1) or UNTD (white; 2) T cells for four days. After four days cells expressing peptides targeted by the TCR are depleted relative to the UNTD T cell screens. DNA is then extracted and DNA encoding the minigene region is PCR amplified prior to NGS to compare minigene abundance. FIG. 1D shows a volcano plot representing data from three sets of screens using T cells from three different donors. P-values were calculated using a paired T test. Peptides above and to the left of the red dotted lines have P-values less than 0.05 and average log 2 fold changes (log 2FC) less than-1; these peptides are labeled; peptides with a position 5 tryptophan are in bold text. Figure discloses SEQ ID NOS 29, 2, 40, 32, 59, 1, 10, 60, 30-31, and 61-66 respectively, in order of appearance.

FIGS. 2A-2C demonstrate confirmation of 1G4 TCR peptide targets by ELISpot and coculture killing assays. FIG. 2A shows quantification of number of spots in an ELISpot assay for IFNγ to assess reactivity of 1G4 TCR (1), DMF5 (2), CD8 UNTD (3), and CD8-1G4 (4) T cells, using T2 cells pulsed with peptide at 20 μg/mL. All conditions were normalized to WT T2 cells. All conditions were done in triplicate. PHA is a positive control for T cell functionality. NY-ESO-1 was a positive control and MART-1 a negative control for 1G4 T cells. MART-1 is a positive control for DMF5 T cells (which recognize the epitope AAGIGILTV (SEQ ID NO: 26). The black dotted line is at y=1. Peptides in addition to NY-ESO-1, that are reactive with 1G4-T cells are marked with an asterisk (*). Figure discloses SEQ ID NOS 65, 61, 64, 60, 67, 31, 30, 7, 63, 32, 62 and 10, respectively, in order of appearance. FIG. 2B shows a list of peptides identified as cross reactive with the 1G4 TCR by coculture killing and ELISpot assays. NY-ESO-1 peptide is underlined at the top of the list. Positions 5 and 8 are highlighted by parentheses and brackets respectively. Figure discloses SEQ ID NOS 40, 7, 31, 30, 32, 29, 68, and 28, respectively, in order of appearance. FIG. 2C shows specific killing of T2/gluc cells pulsed with peptides and co-cultured with CD4/8 T cells transduced with the 1G4 TCR or mock transduced T cells. Figure discloses SEQ ID NOS 27, 30, 29, 7, 31, 32, and 28, respectively, in order of appearance.

FIGS. 3A-3E demonstrate ELISpot and coculture library screens to compare targets of CD8-1G4 and 1G4a95TS cells. FIG. 3A shows quantification of number of spots in an ELISpot assay for IFNγ to assess reactivity of UNTD CD8 (1), CD8-1G4 (2) and CD8-1G4a95TS (3), and CD4-1G4a95TS (4) cells, using T2 cells pulsed with peptide at 20 μg/mL. All conditions are normalized to MART-1 pulsed T2 cells. All conditions were done in triplicate. PHA was a positive control for T cell functionality. NY-ESO-1 was a positive control and MART-1 a negative control for CD8-1G4 and 1G4a95TS cells. Figure discloses SEQ ID NOS 7, 31, 30, 27, 32, 28, and 29, respectively, in order of appearance. FIGS. 3B-3D show volcano plots representing data from three sets of screens using T cells from three different donors. P-values were calculated using a paired T test. Peptides above and to the left of the red dotted lines have P-values less than 0.05 and average log 2 fold changes (log 2FCs) less than-1. Peptides with an average log 2FC less than-2 and p-value less than 0.05 are labeled. FIG. 3B utilized CD8 T cells expressing 1G4 TCR. Figure discloses SEQ ID NOS 59, 10, 2, 30, 1, 29, 32, respectively, in order of appearance. FIG. 3C utilized CD8 T cells expressing 1G4a95TS. Figure discloses SEQ ID NOS 2, 30, 29, 1, 9, respectively, in order of appearance. FIG. 3D utilized CD4 T cells expressing 1G4 TCR. NY-ESO-1 was not significantly depleted in CD8-1G4a95TS or CD4-1G4 screens is labeled in bold text. Figure discloses SEQ ID NOS 29, 2, 1, 7, 6, 11, and 10, respectively, in order of appearance. FIG. 3E shows the quantification of number of spots in an ELISpot assay for IFNγ to assess reactivity of untreated CD8, CD8-1G4 and CD8-native 1G4-α95: TS cells using T2 cells pulsed with peptide at 20 μg/ml. All conditions were normalized to wildtype (WT) T2 cells. All conditions were done in triplicate. PHA is a positive control for T cell functionality. NYESO-1 is a positive and MART-1 a negative control for 1G4 (affinity enhanced and native) T cells. SLLLWISGA (SEQ ID NO: 7) was previously identified as a CD8-dependent target of 1G4 and as not a target for native 1G4-α95: TS cells. The black dotted line is at y=1. Figure discloses SEQ ID NOS 7-9, respectively, in order of appearance.

FIGS. 4A-4J demonstrate ELISpot assays identify CD8-independent targets of 1G4 TCR. FIG. 4A shows quantification of number of spots in an ELISpot assay for IFNγ to assess reactivity of CD4/8-1G4 (1), CD4-1G4 (2), CD8-1G4 (3) and UNTD CD4/8 cells (4), using T2 cells pulsed with peptide at 20 μg/mL. All conditions were normalized to WT T2 cells. All conditions were done in triplicate. PHA was a positive control for T cell functionality. NY-ESO-1 was a positive control and MART-1 a negative control for 1G4 TCR expressing T cells. Figure discloses SEQ ID NOS 7, 31, 30, 27, 32, and 28-29, respectively, in order of appearance. FIG. 4B demonstrates flow cytometry of CD8 or CD4 cells with anti-CD8 and anti-CD4 antibodies, with and without blocking with anti-CD8 antibodies, 3B5 and OKT8, or anti-CD4 antibody, OKT4. FIG. 4C shows quantification of number of spots in an ELISpot assay performed as described for FIG. 4A to analyze cross-reactivity of UNTD CD8 (1), CD8-1G4 (2), CD8-1G4 plus 3B5 antibody (3) and CD8-aG4 plus OKT8 antibody (4) cells. Figure discloses SEQ ID NOS 7, 31, 30, 27, 32, and 28-29, respectively, in order of appearance. FIG. 4D shows quantification of number of spots in an ELISpot assay performed as described for FIG. 4A to analyze cross-reactivity of UNTD-CD4 (1), CD4-1G4 (2), and CD4-1G4 plus OKT4 (3) cells. Figure discloses SEQ ID NOS 7, 31, 30, 27, 32, and 28-29, respectively, in order of appearance. FIG. 4E demonstrates use of flow cytometry to analyze CD8 surface expression in CD8 T cells five days after electroporation with four different sgRNAs targeting CD8a (numbered 61, 65, 70 and 73) and Cas9 mRNA compared to mock, where cells were electroporated but without sgRNA or Cas9 mRNA. FIG. 4F shows histogram plots of flow cytometry comparing detected 1G4 expression with NY-ESO-1/A2 tetramer or anti-1G4 beta chain (vb13.1) antibody in CD4/8, CD8 mock, CD8^{-/-} sgRNA 61 and CD8^{-/-} sgRNA 70 cells. FIG. 4G shows quantification of number of spots in an ELISpot assay performed as described for FIG. 4A, comparing CD8-mock (1), CD8-mock 1G4 (2) cells, CD8^{-/-} sgRNA 61 (3), CD8^{-/-} sgRNA 61 1G4 (4), CD8^{-/-} sgRNA 71 (5), and CD8^{-/-} sgRNA 71 1G4 (6) cells. Figure discloses SEQ ID NOS 7, 31, 30, 27, 32, and 28-29, respectively, in order of appearance. FIG. 4H demonstrates flow cytometric analysis of 1G4 expression and CD8 expression in CD8^{-/-} cells. Histogram plots showing expression of CD8 (left) and 1G4 (right) by flow cytometry after CD8α-directed CRISPR/Cas9 knockout and transduction with the 1G4 TCR. CD8 expression (left) was assessed for CD8 UNTD, CD8 1G4, CD8 mock electroporated UNTD, CD8 mock electroporated 1G4, CD8^{-/-} sgRNA 61 UNTD, CD8^{-/-} sgRNA 61 1G4, CD8^{-/-} sgRNA 70 UNTD, and CD8^{-/-} sgRNA 70 1G4 cells. 1G4 expression (right) was assessed for CD8 mock electroporated UNTD, CD8 mock electroporated 1G4, CD8^{-/-} sgRNA 61 UNTD, CD8^{-/-} sgRNA 61 1G4, CD8^{-/-} sgRNA 70 UNTD, and CD8^{-/-} sgRNA 70 1G4 cells. FIG. 4I shows cytotoxicity of GFP/Luc T2 cells pulsed with 10-fold dilutions of CD8-independent peptides by CD8^{-/-} mock (1), CD8^{-/-} mock 1G4 (2), CD8^{-/-} sgRNA 61 (3), CD8^{-/-} sgRNA 61 1G4 (4), CD8^{-/-} sgRNA 70 (5) and CD8^{-/-} sgRNA 70 1G4 (6) cells, normalized to T2 cells only (7). Figure discloses SEQ ID NOS 27, 30, and 29, respectively, in order of appearance. FIG. 4J shows tetramer titration assays using the previously identified CD8-independent peptide, ELSDWIHQL (SEQ ID NO: 27), and the CD8-dependent peptide, SQCMWLMQA (SEQ ID NO: 32), as well as NY-ESO-1 and MART-1 peptides as positive and negative controls respectively. Figure discloses SEQ ID NOS 27 and 32, respectively, in order of appearance.

FIGS. 5A-5F show expression of wild type or mutant CD8a chains in CD4-1G4 cells increases cytotoxicity without increasing cross-reactivity. FIG. 5A shows diagrams of plasmid constructs for expression of 1G4 with CD8α chains. 1G4 TCRα and TCRβ chains are separated by a P2A sequence, CD8α WT and mutant chains are separated from the TCRb chain by an IRES sequence. FIG. 5B shows histograms depicting flow cytometric analysis of CD4 (top left), CD8 (top right) and 1G4 expression (bottom right), determined by staining for Vb13.1. CD4/8 cells are either untransduced or transduced with 1G4 constructs. CD4 cells are either untransduced or transduced with 1G4, 1G4 CD8α WT, 1G4 CD8α M2, or 1G4 CD8α M5 constructs. FIG. 5C demonstrates specific lysis of GFP/T2 cells pulsed with 10-fold dilutions of NY-ESO-1 peptide, by CD4-1G4 (1), CD4 1G4 CD8α WT (2), CD4-1G4 CD8α M2 (3), CD4-1G4 CD8α M5 (4), and CD4/8-1G4 (5) cells. FIGS. 5D-5E show quantification of number of spots in an ELISpot assay for IFNγ using T2 cells pulsed with peptide at 20 μg/mL. All conditions were normalized to WT T2 cells. All conditions were done in triplicate. PHA was a positive control for T cell functionality. NY-ESO-1 was a positive control and MART-1 a negative control for 1G4 expressing T cells. FIG. 5D shows quantification of T cell reactivity of UNTD CD4 (1), CD4-1G4 (2), CD4-1G4 CD8α WT (3), CD4-1G4 CD8α M2 (4) and CD4-1G4 CD8α M5 (5) cells. Figure discloses SEQ ID NOS 7, 31, 30, 27, 32, and 28-29, respectively, in order of appearance. FIG. 5E shows quantification of T cell reactivity of CD8 mock electroporate UNTD (1), CD8 mock electroporated 1G4 (2), CD8α^{-/-} 1G4 (4), CD8α^{-/-} UNTD (3), CD8α^{-/-} 1G4-CD8α WT (5), CD8α^{-/-} 1G4-CD8α M2 (6), and CD8α^{-/-} 1G4-CD8α M5 (7). Figure discloses SEQ ID NOS 7, 31, 30, 27, 32, 28, and 69, respectively, in order of appearance. FIG. 5F shows specific lysis of GFP/T2 cells pulsed with 10-fold dilutions of NY-ESO-1 or the SLLLWISGA (SEQ ID NO: 7) peptide respectively.

FIGS. 6A-6E show numerous peptides are depleted in coculture screens using the NY-ESO-1 biased library in T2 cells and T cells from three donors. FIG. 6A shows counts of individual peptides detected by NGS after cloning of the NY-ESO-1 library into the PresentER-GFP backbone. NY-ESO-1 biased library is library 1. FIG. 6B demonstrates flow cytometry showing the expression of the NY-ESO-1 biased library in T2 cells (right) compared to untransduced T2 cells (left). Cells expressing plasmid from the library were GFP positive. FIGS. 6C-6E show individual screen data using T cells from three different HLA-A02 negative donors. Fraction of each peptide in the 1G4 screen is on the x-axis and fraction in the UNTD screen is on the y-axis. The blue line has a slope of 1, peptides that are depleted in the 1G4 screens drop away from the blue line. Control peptides NY-ESO-1, LLMWITQC (SEQ ID NO: 1), and LAMWITQC (SEQ ID NO: 2) are labeled in bold text. Peptides with a log 2FC less than-1.5 and fraction over 1e-5 are labeled in regular text. FIG. 6C discloses SEQ ID NOS 10, 2, 70, 1, 32, 71, 7, 72, and 62, respectively, in order of appearance. FIG. 6D discloses SEQ ID NOS 10, 2, 70, 1, 32, 7, 29, and 62, respectively, in order of appearance. FIG. 6E discloses SEQ ID NOS 10, 1, 73, 2, 74, 32, 75-77, 7, 78-79, 29, 80-84, 62, 85-87, respectively, in order of appearance.

FIG. 7 shows previously identified off-target peptide of 1G4 T-cells was not validated by ELISpot. Quantification of number of spots in an ELISpot assay for IFNγ to assess reactivity of 1G4 TCR (1) and DMF5 (2) T cells, using T2 cells pulsed with peptide at 20 μg/mL. All conditions were normalized to WT T2 cells. All conditions were done in triplicate. PHA was a positive control for T cell functionality. NY-ESO-1 was a positive control and MART-1 was a negative control for 1G4 TCR T cells. MART-1 was a positive control for DMF5 T cells. Peptides boxed in were identified as off-targets in CD4/8-1G4 screens and by ELISpot. Peptide NVLLWITAL (SEQ ID NO: 3), boxed in with text bolded, was identified as an off-target of 1G4 TCR T cells in other studies but not in screens of the present disclosure. Figure discloses SEQ ID NOS 59, 63, 32, 62, 3, and 29, respectively, in order of appearance.

FIG. 8 shows a summary of peptides identified as ‘hits’ from the NY-ESO-1 biased library. Peptides identified as hits are listed. Binding affinity predictions to HLA*A02 were made using netMHC 4.0. P-values were calculated using a paired T test. Average Log 2FC and P-value from screens using CD4/8-1G4, CD8-1G4, CD4-1G4 and native CD8-1G4a95TS cells are listed. Both log 2FC and P-values are bolded if Log 2FC is less than-1 and p-value is less than 0.05. Values were marked as not detected (ND) if the peptide was not detected by PCR in screens from all three donors. ELISpot reactivity was assayed through pulsing synthetic peptide in excess onto the surface of T2 cells. If reactivity over background was detected it was marked as yes (Y) and if not detected as no (N). Peptides that were not assayed by ELISpot for the T cells was marked as not tested (NT). Figure discloses SEQ ID NOS 1, 2, 7, 31, 30, 32, 29, 62, 10, 60, 65, 61, 63, 66, 64, 27, 28, 9, 8, 6, and 11, respectively, in order of appearance.

FIGS. 9A-9F demonstrate more peptides are depleted in NY-ESO-1 biased library screens using 1G4 CD8 T cells than native 1G4a95TS CD8 T cells. Individual screen data using T cells from three different HLA-A02 negative donors. Fraction of each peptide in the screen using TCR (either 1G4 or native 1G4a95TS) T cells is on the x-axis and the UNTD T cells on the y-axis. The blue line has a slope of 1, peptides that were depleted in the 1G4 screens drop away from the blue line. Control peptides NY-ESO-1, LLMWITQC (SEQ ID NO: 1), and LAMWITQC (SEQ ID NO: 2) are labeled in bolded text. Peptides with a log 2FC less than-2 and fraction over 1e-5 are labeled in regular text. FIGS. 9A-9C show CD8 T cells transduced with 1G4 TCR. FIGS. 9D-9F show CD8 T cells transduced with native 1G4a95TS TCR. FIG. 9A discloses SEQ ID NOS 88, 2, 89, 7, 32, and 29, respectively, in order of appearance. FIG. 9B discloses SEQ ID NOS 7, 2, 10, 1, 90, 32, and 29, respectively, in order of appearance. FIG. 9C discloses SEQ ID NOS 10, 2, 7, 1, 32, and 29, respectively, in order of appearance. FIG. 9D discloses SEQ ID NOS 1, 2, 91, and 29, respectively, in order of appearance. FIG. 9E discloses SEQ ID NOS 2, 1, and 29, respectively, in order of appearance. FIG. 9F discloses SEQ ID NOS 1, 2, 90, 92-93, and 29, respectively, in order of appearance.

FIGS. 10A-10D show net cytotoxicity of NY-ESO-1 pulsed T2 cells by CD8-1G4 cells is decreased after blocking with anti-CD8 antibodies and Fab. Cytotoxicity of GFP/Luc T2 cells pulsed with 10-fold dilutions of NY-ESO-1 peptide by CD8-1G4 or CDB-UNTD cells with or without preincubation with CD8 blocking antibodies at 24 hours, normalized to T2 cells only. FIG. 10A shows total specific lysis of target cells by CD8-UNTD (1) and CD8-1G4 (2), or after preincubation with anti CD8 antibodies 3B5 (3 and 4) and OKT8 (5). FIG. 10B shows net cytotoxicity of data represented in FIG. 10A. Specific lysis of CD8-1G4 cells minus matched CD8-UNTD cells. Specific lysis was done without preincubation with antibodies (1), or after preincubation with anti-CD8 antibody 3B5 (2) or OKT8 (3). FIG. 10C shows total specific lysis by CD8-1G4 cells (2), CD8-UNTD cells (1), CD8-1G4 plus 3B5 antibody (4), CD8-UNTD plus 3B5 antibody (3), CD8-1G4 plus Fab (6) and CD8-UNTD plus Fab (5). FIG. 10D shows net cytotoxicity of data represented in FIG. 10C. Net specific lysis in the absence of blocking antibodies (1), or after preincubation with anti-CD8 antibody 3B5 (2) or fab (3).

FIG. 11A demonstrates CD8 is needed for maximal NY-ESO-1/HLA-A02 tetramer binding to 1G4 TCR. Flow cytometry of CD8-UNTD or CD8-1G4 stained with NY-ESO-1/HLA*A02 tetramer with and without preincubation with anti-CD8 antibody 3B5. Binding of 3B5 was confirmed with FITC conjugated anti-mouse secondary antibody. FIG. 11B demonstrates blocking CD3 does not affect 1G4 tetramer staining. Flow cytometry of CD8-UNTD (top) or CD8-1G4 (bottom) cells stained with NY-ESO-1/A02 tetramer with (middle) and without (left) preincubation with anti-CD3 antibody OKT3. Binding of OKT3 was confirmed with FITC conjugated anti-mouse secondary antibody (right).

FIGS. 12A-12C demonstrate CD4-1G4 cells from three donors deplete multiple peptides in coculture screens using the NY-ESO-1 biased library in T2. Individual screen data using CD4 T cells from three different HLA-A02 negative donors. Fraction of each peptide in the 1G4 screen is on the x-axis and fraction in the UNTD screen is on the y-axis. The blue line has a slope of 1, peptides that are depleted in the 1G4 screens drop away from the blue line. Control peptides NY-ESO-1, LLMWITQC (SEQ ID NO: 1), and LAMWITQC (SEQ ID NO: 2) are labeled in bold text. Peptides with a log 2FC less than-2 and fraction over 1e-5 are labeled in regular text. FIG. 12A discloses SEQ ID NOS 2, 7, 1, 10, and 94, respectively, in order of appearance. FIG. 12B discloses SEQ ID NOS 7, 10, 2, 95, 1, 96-97, and 29, respectively, in order of appearance. FIG. 12C discloses SEQ ID NOS 2, 1, and 29, respectively, in order of appearance.

FIG. 13 shows comparison of peptide ranking methods for identified peptide hits of 1G4 TCR. Table of peptides identified as hits for 1G4 T cells. Blosum62 scoring, using a gap penalty of 1 and extension penalty of 4, was performed retroactively for all peptides in the NY-ESO-1 biased library. PMBEC calculations presented here were only performed on 9mer peptides in the library while Blosum62 calculations were performed on all peptides of any length. Based on either PMBEC or Blosum62 score peptides were then ranked, with most similar peptides getting lower ranks i.e., NY-ESO-1 peptide ranks 1. Peptides with tied scores were given the same lowest rank. Percent was calculated by dividing rank, where most similar peptides are ranked highest, divided by total number of peptides (n) in the library. Figure discloses SEQ ID NOS 40, 7, 31, 30, 32, 29, 10, and 27-28, respectively, in order of appearance.

FIG. 14 shows MAGE-A3/HLA-A01 biased peptide library design includes previously unpredicted cross-reactive peptide from Titin. Binding affinity for all 9mer peptides in the human proteome to HLA-A01 was predicted with netMHC 4.0. Peptides with predicted binding below 500 nM were then scored for similarity to MAGE-A3 peptide (EVDPIGHLY, SEQ ID NO: 4) using either PMBEC scoring algorithm or Blosum62, with gap and extension penalties of 1 and 4 respectively, scoring algorithm. Peptides were then ranked, with most similar peptides getting lower ranks i.e., MAGE-A3 peptide ranks 1. Peptides with tied scores were given the same lowest rank. All peptides with rank below 5000 were included in library design. Percent was calculated by dividing rank, where most similar peptides are ranked highest divided by total number of peptides in the library. Figure discloses SEQ ID NOS 4 and 5, respectively, in order of appearance.

FIG. 15 shows ELISpot assay confirmation of insignificantly but robustly depleted peptide hits of the Affinity Enhanced 1G4 TCR. Quantification of number of spots in an ELISpot assay for IFNγ to assess reactivity of 1G4 (1) and DMF5 (2) T cells, using T2 cells pulsed with peptide at 20 μg/mL. All conditions were normalized to wildtype (WT T2) cells. All conditions were done in triplicate. PHA was a positive control for T cell functionality. NY-ESO-1 was a positive and MART-1 a negative control for 1G4 T cells. MART-1 was a positive control for DMF5 T cells. The black dotted line is at y=1. Peptides, in addition to NY-ESO-1, that are reactive with 1G4 T cells are boxed. Figure discloses SEQ ID NOS 27-28 and 98-99, respectively, in order of appearance.

FIG. 16 shows additional off target peptide hits of the native 1G4 TCR were not confirmed via ELISpot assay. Quantification of number of spots in an ELISpot assay for IFNγ to assess reactivity of UNTD CD8 (1), CD8-1G4 (2) and CD8-native 1G4-α95: TS (3) cells, using T2 cells pulsed with peptide at 20 μg/mL. All conditions were normalized to wildtype (WT) T2 cells. All conditions were done in triplicate. PHA was a positive control for T cell functionality. NY-ESO-1 was a positive and MART-1 a negative control for CD8-1G4 and native 1G4-α95: TS cells. SLLLWISGA (SEQ ID NO: 7) is a previously identified target of CD8-1G4 but not CD8-native 1G4-α95: TS cells. Peptides VLIDWINDV (SEQ ID NO: 8) and HTWERMWMHV (SEQ ID NO: 9) were identified as peptide hits in CD8-native 1G4-α95: TS but not CD8-1G4 coculture screens. The black dotted line is at y=1.

FIG. 17 shows additional off target peptide hits of the CD4-1G4 cells were not confirmed via ELISpot assay. Quantification of number of spots in an ELISpot assay for IFNγ to assess reactivity of CD8 UNTD (1), CD8-1G4 (2), CD4 UNTD (3), and CD4-1G4 (4) cells, using T2 cells pulsed with peptide at 20 μg/mL. All conditions were normalized to wildtype (WT) T2 cells. All conditions were done in triplicate. PHA was a positive control for T cell functionality. NY-ESO-1 was a positive and MART-1 a negative control for 1G4 T cells. SLLLWISGA (SEQ ID NO: 7) was previously identified CD8-dependent target of 1G4. QVGMWVWEA (SEQ ID NO: 10) was identified in 1G4/NY-ESO-1 library coculture screens but did not previously validate by ELISpot. Peptides LILSYIAGI (SEQ ID NO: 11) and DLLNHIATV (SEQ ID NO: 6) were identified as peptide hits in CD4-1G4 but not CD8-1G4 coculture screens.

FIG. 18 shows expression of 1G4 constructs with and without CD8α in CD8^{-/-} cells. Histograms depicting 1G4 (left) and CD8 (right) expression as determined by flow cytometry of CD8 cells mock electroporated (electroporated without sgRNA or Cas9) and UNTD (1) or transduced with 1G4 (2), and electroporated with sgRNA 61 and Cas9 and UNTD (3) or transduced with 1G4 (4), 1G4-CD8a WT (5), 1G4-CD8a M2 (6), and 1G4-CDa8 M5 (7). For cells electroporated with sgRNA and Cas9, CD8a−/− cells were isolated prior to transduction.

FIG. 19 shows mutant CD8α does not restore CD8^{-/-} 1G4 T cell cross-reactivity. Reactivity of CD8α^{-/-} 1G4 (1), CD8α^{-/-} 1G4-CD8α^{-/-} WT (2), CD8α 1G4-CD8α^{-/-} M2 (3), and CD8α^{-/-} 1G4-CD8α M5 (4) T cells is shown. The black dotted line is at y=1. Peptides in addition to NY-ESO-1, that are reactive with CD8α 1G4 cells are boxed. Figure discloses SEQ ID NOS 7, 31, 30, 27, 32, and 28-29, respectively, in order of appearance.

FIG. 20 shows INFγ ELISpot using T cells from donor 5 and T2 cells pulsed with peptide. Peptides that were previously shown to be CD8-independent are: SEQ ID NO: 30 (abbreviated as TQIQ (SEQ ID NO: 100)), SEQ ID NO: 29 (abbreviated as VVN) and SEQ ID NO: 27 (abbreviated as ELS). Peptides that were found to require CD8 are: SEQ ID NO: 7 (abbreviated as SLL), SEQ ID NO: 31 (abbreviated as TLL), SEQ ID NO: 32 (abbreviated as SQC) and SEQ ID NO: 28 (abbreviated as LVM). MART-1 was the control peptide. PHA was used for positive control of INFγ release.

FIG. 21 shows ELIspot close-up of peptides that were previously shown to require CD8 to elicit a response in T cells. In CD8 beta knock out T cells, reactivity to these peptides is reduced.

FIGS. 22A-22B show flow cytometry data to demonstrate successful knock out of the CD8β chain of CD4 negative T cells.

FIGS. 23A-23B show flow cytometry data to demonstrate MACS separation of guide 4 electroporated CD4 negative T cells in CD8αβ T cells and CD8αα expressing T cells (top row). Transduction efficiency of 1G4 was not affected by separating cells (bottom row).

FIGS. 24A-24E demonstrate that DMF5 expressing T cells have reduced off target reactivity in the absence of CD8 binding. FIG. 24A shows quantification of number of spots in an ELISpot assay for IFNγ to assess reactivity of untransduced CD4/8, CD4/8 DMF5, untransduced CD4, CD4 DMF5, untransduced CD8, and CD8 DMF5 cells using T2 cells pulsed with peptide 20 μg/ml. All conditions were normalized to WT T2 cells. All conditions were done in triplicate. PHA is a positive control for T cell functionality. MART-1 is a positive and NY-ESO-1 a negative control for DMF5 expressing T cells. The black dotted line is at y=1. Figure discloses SEQ ID NOS 43, 45, 101-103, 44, 46, and 104-105, respectively, in order of appearance. FIGS. 24B-24E show specific lysis of GFP/T2 cells pulsed with 10-fold dilutions of MART-1 (FIG. 24B) or the off target peptides SMAGIGIVDV (SEQ ID NO: 43) (FIG. 24C), NLSNLGILPV (SEQ ID NO: 44) (FIG. 24D), and SMLGIGIVPV (SEQ ID NO: 45) (FIG. 24E) respectively.

FIG. 25 shows a schematic overview of engineered cell types used in the Examples described herein. Cell types used include CD8 T cells expressing wild type CD8, CD8 T cells transduced with the 1G4 TCR, CD8αKO 1G4 T cells whereby CRISPR/Cas9 is used to knock out endogenous CD8α, CD8αKO T cells transduced with 1G4 and mutant CD8α and CD4 T cells expressing wildtype CD4.

### DETAILED DESCRIPTION

It is to be appreciated that certain aspects, modes, embodiments, variations and features of the present methods are described below in various levels of detail in order to provide a substantial understanding of the present technology.

In practicing the present methods, many conventional techniques in molecular biology, protein biochemistry, cell biology, immunology, microbiology and recombinant DNA are used. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology. Methods to detect and measure levels of polypeptide gene expression products (i.e., gene translation level) are well-known in the art and include the use of polypeptide detection methods such as antibody detection and quantification techniques. (See also, Strachan & Read, Human Molecular Genetics, Second Edition. (John Wiley and Sons, Inc., NY, 1999)).

Typical therapeutic anti-cancer monoclonal antibodies (mAb), like those that bind to CD19, and Chimeric Antigen Receptor T cells (CAR T cells) recognize cell surface proteins. Cell surface proteins constitute only a tiny fraction of the cellular protein content. Most mutated or oncogenic tumor associated proteins are typically nuclear or cytoplasmic, preventing use of canonical anti-cancer mAbs and CAR-T cells to target such mutated or oncogenic tumor associated proteins. In certain instances, these intracellular proteins can be degraded in the proteasome, processed, and presented on the cell surface by MHC class I molecules as T cell epitopes that are recognized by T-cell receptors (TCRs). These peptides can be derived from proteins from any cellular location, thus vastly expanding the repertoire of potentially targetable cancer antigens. However, TCR cross-reactivities with presented peptides that have similar amino acid sequences are frequently observed. One challenge of TCR-based therapies is that it is extremely difficult to predict off-target reactivities that can lead to toxicities or to modulate such detrimental cross-reactions. Therefore, methods to modulate TCR specificity are increasingly useful in, for example, the treatment of subjects in need thereof (e.g., subjects with cancer) using TCR-based therapies.

The present disclosure surprisingly demonstrates how CD4 and CD8 coreceptors influence TCR (e.g., 1G4 TCR) cross-reactivities and provides compositions and methods of leveraging of such activity for therapeutic benefit. The CD8 coreceptor can engage with MHC I, allowing MHC I restricted TCRs to respond to lower affinity interactions and at lower peptide-MHC (pMHC densities), leading to an increase in cross-reactivity risk in CD8 cells. However, CD4 expressing MHCI restricted TCRs require higher affinity interactions and CD8-independence to be activated. Recruitment of CD4 cells in addition to CD8 cells enhances the anticancer effect of T cell therapies.

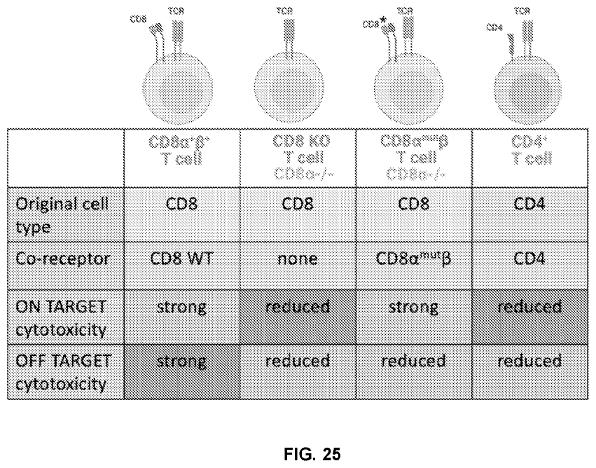

The present disclosure demonstrates, among other things, that through mutation of CD8α, off-target reactivity with certain CD8-dependent peptides in CD4 and CD8 cells could be reduced without sacrificing on-target cytotoxicity. For example, CD4 T cells expressing the affinity enhanced 1G4 TCR (CD4-1G4) killed target cells presenting the NY-ESO-1 peptide, as well as cells presenting a smaller subset of cross-reactive peptides recognized by CD8 T cells expressing the affinity enhanced 1G4 TCR (CD8-1G4). In addition, CD8α^{-/-} cells expressing a mutant CD8α chain had decreased cross-reactivity, while maintaining on target reactivity, providing a possible strategy to generate safer TCR therapies (FIG. 25). Finally, this phenomenon was not limited to the 1G4 TCR but could be successfully extrapolated to other CD8-independent therapeutic TCRs, such as DMF5.

Accordingly, technologies of the present disclosure provide, among other things, engineered immune cells with improved TCR specificity and decreased associated toxicity of TCR-T cells. The compositions and methods disclosed herein do not rely on altering the TCR sequence itself, and maintain the cytotoxic anti-cancer potential of these cells. Such an approach might be applied generally to other therapeutic TCRs without the need to change the underlying TCR therapeutic agent.

### Definitions

Unless defined otherwise, all technical and scientific terms used herein generally have the same meaning as commonly understood by one of ordinary skill in the art to which this technology belongs. As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, up to 10%, up to 5%, or up to 1% of a given value. Alternatively, particularly with respect to biological systems or processes, the term can mean within an order of magnitude, within 5-fold, or within 2-fold, of a value.

As used herein, the “administration” of an agent (e.g., engineered immune cells as described herein or drug) to a subject includes any route of introducing or delivering to a subject the agent to perform its intended function. Administration can be carried out by any suitable route, including but not limited to, orally, intranasally, parenterally (intravenously, intramuscularly, intraperitoneally, or subcutaneously), rectally, intrathecally, intratumorally or topically. Administration includes self-administration and the administration by another.

As used herein “adoptive cell therapeutic composition” refers to any composition comprising cells suitable for adoptive cell transfer. In exemplary embodiments, the adoptive cell therapeutic composition comprises a cell type including, for example, TCR (i.e., heterologous T-cell receptor) modified lymphocytes (e.g., eTCR T cells and caTCR T cells). In another embodiment, the adoptive cell therapeutic composition comprises T cells. In another embodiment, T-cells form the adoptive cell therapeutic composition.

The term “amino acid” refers to naturally occurring and non-naturally occurring amino acids, as well as amino acid analogs and amino acid mimetics that function in a manner similar to the naturally occurring amino acids. Naturally encoded amino acids are the 20 common amino acids (alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine) and pyrolysine and selenocysteine. Amino acid analogs refer to agents that have the same basic chemical structure as a naturally occurring amino acid, i.e., an a carbon that is bound to a hydrogen, a carboxyl group, an amino group, and an R group, such as, homoserine, norleucine, methionine sulfoxide, methionine methyl sulfonium. Such analogs have modified R groups (such as, norleucine) or modified peptide backbones, but retain the same basic chemical structure as a naturally occurring amino acid. In some embodiments, amino acids forming a polypeptide are in the D form. In some embodiments, the amino acids forming a polypeptide are in the L form. In some embodiments, a first plurality of amino acids forming a polypeptide are in the D form, and a second plurality of amino acids are in the L form.

Amino acids are referred to herein by either their commonly known three letter symbols or by the one-letter symbols recommended by the IUPAC-IUB Biochemical Nomenclature Commission. Nucleotides, likewise, are referred to by their commonly accepted single-letter code.

As used herein, the term “antibody” collectively refers to immunoglobulins or immunoglobulin-like molecules including by way of example and without limitation, IgA, IgD, IgE, IgG and IgM, combinations thereof, and similar molecules produced during an immune response in any vertebrate, for example, in mammals such as humans, goats, rabbits and mice, as well as non-mammalian species, such as shark immunoglobulins. As used herein, “antibodies” (includes intact immunoglobulins) and “antigen binding fragments” specifically bind to a molecule of interest (or a group of highly similar molecules of interest) to the substantial exclusion of binding to other molecules (for example, antibodies and antibody fragments that have a binding constant for the molecule of interest that is at least 10³ M-1 greater, at least 10⁴ M-1 greater or at least 10⁵ M-1 greater than a binding constant for other molecules in a biological sample). The term “antibody” also includes genetically engineered forms such as chimeric antibodies (for example, humanized murine antibodies), heteroconjugate antibodies (such as, bispecific antibodies). See also, Pierce Catalog and Handbook, 1994-1995 (Pierce Chemical Co., Rockford, Ill.); Kuby, J., Immunology, 3rd Ed., W.H. Freeman & Co., New York, 1997.

More particularly, an antibody refers to a polypeptide ligand comprising at least a light chain immunoglobulin variable region or heavy chain immunoglobulin variable region which specifically recognizes and binds an epitope of an antigen. Antibodies are composed of a heavy and a light chain, each of which has a variable region, termed the variable heavy (VH) region and the variable light (VL) region. Together, the VH region and the VL region are responsible for binding the antigen recognized by the antibody. Typically, an immunoglobulin has heavy (H) chains and light (L) chains interconnected by disulfide bonds. There are two types of light chain, lambda (λ) and kappa (κ). There are five main heavy chain classes (or isotypes) which determine the functional activity of an antibody molecule: IgM, IgD, IgG, IgA and IgE. Each heavy and light chain contains a constant region and a variable region, (the regions are also known as “domains”). In combination, the heavy and the light chain variable regions specifically bind the antigen. Light and heavy chain variable regions contain a “framework” region interrupted by three hypervariable regions, also called “complementarity-determining regions” or “CDRs”. The extent of the framework region and CDRs have been defined (see, Kabat et al., Sequences of Proteins of Immunological Interest, U.S. Department of Health and Human Services, 1991, which is hereby incorporated by reference). The Kabat database is now maintained online. The sequences of the framework regions of different light or heavy chains are relatively conserved within a species. The framework region of an antibody, that is the combined framework regions of the constituent light and heavy chains, largely adopt a β-sheet conformation and the CDRs form loops which connect, and in some cases form part of, the β-sheet structure. Thus, framework regions act to form a scaffold that provides for positioning the CDRs in correct orientation by inter-chain, non-covalent interactions.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a VH CDR3 is located in the variable domain of the heavy chain of the antibody in which it is found, whereas a VL CDR1 is the CDR1 from the variable domain of the light chain of the antibody in which it is found. Antibodies with different specificities (i.e., different combining sites for different antigens) have different CDRs. Although it is the CDRs that vary from antibody to antibody, only a limited number of amino acid positions within the CDRs are directly involved in antigen binding. These positions within the CDRs are called specificity determining residues (SDRs). An antibody or antigen binding fragment thereof specifically binds to an antigen.

As used herein, an “antigen” refers to a molecule to which an immunoglobulin-related composition (e.g., antibody or antigen binding fragment thereof or T Cell Receptor) can selectively bind. The target antigen may be a protein, carbohydrate, nucleic acid, lipid, hapten, or other naturally occurring or synthetic compound. In some embodiments, the target antigen may be a peptide/MHC complex. An antigen may also be administered to an animal to generate an immune response in the animal.

The term “antigen binding fragment” refers to a fragment of the whole immunoglobulin structure which possesses a part of a polypeptide responsible for binding to antigen. Examples of the antigen binding fragment include scFv, (scFv)2, scFvFc, Fab, Fab′ and F(ab′)2, but are not limited thereto.

By “binding affinity” is meant the strength of the total noncovalent interactions between a single binding site of a molecule (e.g., an immunoglobulin-related composition, TCR) and its binding partner (e.g., an antigen). The affinity of a molecule X for its partner Y can generally be represented by the dissociation constant (K_{d}). Affinity can be measured by standard methods known in the art, including those described herein. A low-affinity complex contains an immunoglobulin-related composition that generally tends to dissociate readily from the antigen, whereas a high-affinity complex contains an immunoglobulin-related composition that generally tends to remain bound to the antigen for a longer duration.

As used herein, the term “biological sample” means sample material derived from living cells. Biological samples may include tissues, cells, protein or membrane extracts of cells, and biological fluids (e.g., ascites fluid or cerebrospinal fluid (CSF)) isolated from a subject, as well as tissues, cells and fluids present within a subject. Biological samples of the present technology include, but are not limited to, samples taken from breast tissue, renal tissue, the uterine cervix, the endometrium, the head or neck, the gallbladder, parotid tissue, the prostate, the brain, the pituitary gland, kidney tissue, muscle, the esophagus, the stomach, the small intestine, the colon, the liver, the spleen, the pancreas, thyroid tissue, heart tissue, lung tissue, the bladder, adipose tissue, lymph node tissue, the uterus, ovarian tissue, adrenal tissue, testis tissue, the tonsils, thymus, blood, hair, buccal, skin, serum, plasma, CSF, semen, prostate fluid, seminal fluid, urine, feces, sweat, saliva, sputum, mucus, bone marrow, lymph, and tears. Biological samples can also be obtained from biopsies of internal organs or from cancers. Biological samples can be obtained from subjects for diagnosis or research or can be obtained from non-diseased individuals, as controls or for basic research. Samples may be obtained by standard methods including, e.g., venous puncture and surgical biopsy. In certain embodiments, the biological sample is a tissue sample obtained by needle biopsy.

As used herein, a “cancer” is a disease state characterized by the presence in a subject of cells demonstrating abnormal uncontrolled replication and in some aspects, the term may be used interchangeably with the term “tumor.” The term “cancer or tumor antigen” refers to an antigen known to be associated and expressed in a cancer cell or tumor cell or tissue, and the term “cancer or tumor targeting antibody” refers to an antibody that targets such an antigen. In some embodiments, the cancer or tumor antigen is not expressed in a non-cancer cell or tissue. In some embodiments, the cancer or tumor antigen is expressed in a non-cancer cell or tissue at a level significantly lower compared to a cancer cell or tissue.

In some embodiments, the cancer is selected from: circulatory system, for example, heart (sarcoma [angiosarcoma, fibrosarcoma, rhabdomyosarcoma, liposarcoma], myxoma, rhabdomyoma, fibroma, and lipoma), mediastinum and pleura, and other intrathoracic organs, vascular tumors and tumor-associated vascular tissue; respiratory tract, for example, nasal cavity and middle ear, accessory sinuses, larynx, trachea, bronchus and lung such as small cell lung cancer (SCLC), non-small cell lung cancer (NSCLC), bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; gastrointestinal system, for example, esophagus (squamous cell carcinoma, adenocarcinoma, leiomyosarcoma, lymphoma), stomach (carcinoma, lymphoma, leiomyosarcoma), gastric, pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma); gastrointestinal stromal tumors and neuroendocrine tumors arising at any site; genitourinary tract, for example, kidney (adenocarcinoma, Wilm's tumor [nephroblastoma], lymphoma, leukemia), bladder and/or urethra (squamous cell carcinoma, transitional cell carcinoma, adenocarcinoma), prostate (adenocarcinoma, sarcoma), testis (seminoma, embryonal carcinoma, teratocarcinoma, choriocarcinoma, sarcoma, interstitial cell carcinoma, fibroma, fibroadenoma, adenomatoid tumors, lipoma); liver, for example, hepatoma (hepatocellular carcinoma), cholangiocarcinoma, hepatoblastoma, angiosarcoma, hepatocellular adenoma, hemangioma, pancreatic endocrine tumors (such as pheochromocytoma, insulinoma, vasoactive intestinal peptide tumor, islet cell tumor and glucagonoma); bone, for example, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; nervous system, for example, neoplasms of the central nervous system (CNS), primary CNS lymphoma, skull cancer (osteoma, hemangioma, granuloma, xanthoma, osteitis deformans), meninges (meningioma, meningiosarcoma, gliomatosis), brain cancer (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma); reproductive system, for example, gynecological, uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), placenta, vagina (clear cell carcinoma, squamous cell carcinoma, botryoid sarcoma (embryonal rhabdomyosarcoma), fallopian tubes (carcinoma) and other sites associated with female genital organs, penis, prostate, testis, and other sites associated with male genital organs; hematologic system, for example, blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma]; oral cavity, for example, lip, tongue, gum, floor of mouth, palate, and other parts of mouth, parotid gland, and other parts of the salivary glands, tonsil, oropharynx, nasopharynx, pyriform sinus, hypopharynx, and other sites in the lip, oral cavity and pharynx; skin, for example, malignant melanoma, cutaneous melanoma, basal cell carcinoma, squamous cell carcinoma, Kaposi's sarcoma, moles dysplastic nevi, lipoma, angioma, dermatofibroma, and keloids; adrenal glands: neuroblastoma; and other tissues comprising connective and soft tissue, retroperitoneum and peritoneum, eye, intraocular melanoma, and adnexa, breast, head or neck, anal region, thyroid, parathyroid, adrenal gland and other endocrine glands and related structures, secondary and unspecified malignant neoplasm of lymph nodes, secondary malignant neoplasm of respiratory and digestive systems and secondary malignant neoplasm of other sites. In some embodiments, the cancer is a colon cancer, colorectal cancer or rectal cancer. In some embodiments, the cancer is a lung cancer. In some embodiments, the cancer is a pancreatic cancer. In some embodiments, the cancer is an adenocarcinoma, an adenocarcinoma, an adenoma, a leukemia, a lymphoma, a carcinoma, a melanoma, an angiosarcoma, or a seminoma.

In some embodiments, the cancer is a solid tumor. In other embodiments, the cancer is not a solid tumor. In some embodiments, the cancer is from a carcinoma, a sarcoma, a myeloma, a leukemia, or a lymphoma. In some embodiments, the cancer is a primary cancer or a metastatic cancer. In some embodiments, the cancer is a relapsed cancer. In some embodiments, the cancer reaches a remission, but can relapse. In some embodiments, the cancer is unresectable.

As used herein, the term “conservative sequence modification” refers to an amino acid modification that does not significantly affect or alter the binding characteristics of a particular polypeptide comprising the amino acid sequence. Conservative modifications can include amino acid substitutions, additions, and deletions. Modifications can be introduced into the presently disclosed technologies by standard techniques known in the art, such as site-directed mutagenesis and PCR-mediated mutagenesis. Amino acids can be classified into groups according to their physicochemical properties such as charge and polarity. Conservative amino acid substitutions are ones in which the amino acid residue is replaced with an amino acid within the same group. For example, amino acids can be classified by charge: positively-charged amino acids include lysine, arginine, histidine; negatively-charged amino acids include aspartic acid and glutamic acid; and neutral charge amino acids include alanine, asparagine, cysteine, glutamine, glycine, isoleucine, leucine, methionine, phenylalanine, proline, serine, threonine, tryptophan, tyrosine, and valine. In addition, amino acids can be classified by polarity: polar amino acids include arginine (basic polar), asparagine, aspartic acid (acidic polar), glutamic acid (acidic polar), glutamine, histidine (basic polar), lysine (basic polar), serine, threonine, and tyrosine; non-polar amino acids include alanine, cysteine, glycine, isoleucine, leucine, methionine, phenylalanine, proline, tryptophan, and valine. Thus, one or more amino acid residues within a certain region can be replaced with other amino acid residues from the same group and the altered protein can be tested for retained function (i.e., the functions set forth in (c) through (l) above) using the functional assays described herein. In certain embodiments, no more than one, no more than two, no more than three, no more than four, no more than five residues within a specified sequence are altered.

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease, a positive control (a compound or composition known to exhibit the desired therapeutic effect) and a negative control (a subject or a sample that does not receive the therapy or receives a placebo) are typically employed.

As used herein, the term “effective amount” refers to a quantity sufficient to achieve a desired therapeutic and/or prophylactic effect, e.g., an amount which results in the prevention of, or a decrease in a disease and/or condition described herein or one or more signs or symptoms associated with a disease and/or condition described herein. In the context of therapeutic or prophylactic applications, the amount of a composition administered to the subject will vary depending on the composition, the degree, type, and severity of the disease and on the characteristics of the individual, such as general health, age, sex, body weight and tolerance to drugs. The skilled artisan will be able to determine appropriate dosages depending on these and other factors. The compositions can also be administered in combination with one or more additional therapeutic compounds. In the methods described herein, the therapeutic compositions may be administered to a subject having one or more signs or symptoms of a disease or condition described herein. As used herein, a “therapeutically effective amount” of a composition refers to composition levels in which the physiological effects of a disease or condition are ameliorated or eliminated. A therapeutically effective amount can be given in one or more administrations.

As used herein, the term “epitope” means an antigenic determinant capable of specific binding to an immunoglobulin-related composition such as an antibody. Epitopes usually consist of chemically active surface groupings of molecules such as amino acids or sugar side chains and usually have specific three dimensional structural characteristics, as well as specific charge characteristics. Conformational and non-conformational epitopes are distinguished in that the binding to the former but not the latter is lost in the presence of denaturing solvents. In some embodiments, an “epitope” is a region of the target antigen to which TCR compositions of the present technology specifically bind. In some embodiments, the epitope is a conformational epitope or a non-conformational epitope. Epitope mapping can be performed by methods known in the art.

As used herein, “expression” includes one or more of the following: transcription of the gene into precursor mRNA; splicing and other processing of the precursor mRNA to produce mature mRNA; mRNA stability; translation of the mature mRNA into protein (including codon usage and tRNA availability); and glycosylation and/or other modifications of the translation product, if required for proper expression and function.

As used herein, an “expression control sequence” refers to polynucleotide sequences which are necessary to affect the expression of coding sequences to which they are operably linked. Expression control sequences are sequences which control the transcription, post-transcriptional events and translation of nucleic acid sequences. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (e.g., ribosome binding sites); sequences that enhance protein stability; and when desired, sequences that enhance protein secretion. The nature of such control sequences differs depending upon the host organism; in prokaryotes, such control sequences generally include promoter, ribosomal binding site, and transcription termination sequence. The term “control sequences” is intended to encompass, at a minimum, any component whose presence is essential for expression, and can also encompass an additional component whose presence is advantageous, for example, leader sequences.

As used herein, “F(ab)” refers to a fragment of an antibody structure that binds to an antigen but is monovalent and does not have a Fc portion, for example, an antibody digested by the enzyme papain yields two F(ab) fragments and an Fc fragment (e.g., a heavy (H) chain constant region; Fc region that does not bind to an antigen).

As used herein, “F(ab′)2” refers to an antibody fragment generated by pepsin digestion of whole IgG antibodies, wherein this fragment has two antigen binding (ab′) (bivalent) regions, wherein each (ab′) region comprises two separate amino acid chains, a part of a H chain and a light (L) chain linked by an S—S bond for binding an antigen and where the remaining H chain portions are linked together. A “F(ab′)2” fragment can be split into two individual Fab′ fragments.

As used herein, the term “gene” means a segment of DNA that contains all the information for the regulated biosynthesis of an RNA product, including promoters, exons, introns, and other untranslated regions that control expression.

The term “HLA-A2”, as used herein, representatively refers to the subtypes, examples of which include, but are not limited to, HLA-A*02:01, HLA-A*02:02, HLA-A*02:03, HLA-A*02:04, HLA-A*02:05, HLA-A*02:06, HLA-A*02:07, HLA-A*02:10, HLA-A*02:11, HLA-A*02:13, HLA-A*02:16, HLA-A*02:18, HLA-A*02:19, HLA-A*02:28 and HLA-A*02:50.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. A polynucleotide or polynucleotide region (or a polypeptide or polypeptide region) has a certain percentage (for example, at least 40%, 45%, 50%, 55%, 60%, 65%, 70%, 75%, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art. In some embodiments, default parameters are used for alignment. One alignment program is BLAST, using default parameters. In particular, programs are BLASTN and BLASTP, using the following default parameters: Genetic code=standard; filter-none; strand-both; cutoff=60; expect=10; Matrix=BLOSUM62; Descriptions-50 sequences; sort by=HIGH SCORE; Databases=non-redundant, GenBank+EMBL+DDBJ+PDB+GenBank CDS translations+SwissProtein+SPupdate+PIR. Details of these programs can be found at the National Center for Biotechnology Information. Biologically equivalent polynucleotides are those having the specified percent homology and encoding a polypeptide having the same or similar biological activity. Two sequences are deemed “unrelated” or “non-homologous” if they share less than 40% identity, or less than 25% identity, with each other.

As used herein, the terms “identical” or percent “identity”, when used in the context of two or more nucleic acids or polypeptide sequences, refer to two or more sequences or subsequences that are the same or have a specified percentage of amino acid residues or nucleotides that are the same (i.e., about 40%, 45% 50%, 55%, 60%, 65%, 70%, 75%, 80%, 85%, 90%, 91%, 92%, 93%, 94%, 95%, 96%, 97%, 98%, 99%, or higher identity over a specified region (e.g., nucleotide sequence encoding a TCR or CD8 molecule as described herein or amino acid sequence of a TCR or CD8 molecule as described herein)), when compared and aligned for maximum correspondence over a comparison window or designated region as measured using a BLAST or BLAST 2.0 sequence comparison algorithms with default parameters described below, or by manual alignment and visual inspection (e.g., NCBI web site). Such sequences are then said to be “substantially identical.” This term also refers to, or can be applied to, the complement of a test sequence. The term also includes sequences that have deletions and/or additions, as well as those that have substitutions. In some embodiments, identity exists over a region that is at least about 8, 9, 10, 11, 12, 15, 20, 25, 50, 75, 100, 150, 200, 250, 300, 350, 400, 450, 500, 550, 600, 650, 700, 750, 800, 850, 900, 950, 1000 or more amino acids in length. In some embodiments, identity exists over a region that is at least about 21, 24, 27, 30, 33, 36, 45, 60, 75, 150, 225, 300, 450, 600, 750, 900, 1050, 1200, 1350, 1500, 1650, 1800, 1950, 2100, 2250, 2400, 2550, 2700, 2850, 3000 or more nucleotides in length.

As used herein, the term “immune cell” refers to any cell that plays a role in the immune response of a subject. Immune cells are of hematopoietic origin, and include lymphocytes, such as B cells and T cells; natural killer cells; myeloid cells, such as monocytes, macrophages, dendritic cells, eosinophils, neutrophils, mast cells, basophils, and granulocytes. As used herein, the term “engineered immune cell” refers to an immune cell that is genetically modified, and in particular, wherein the immune cell is a T cell. As used herein, the term “native immune cell” refers to an immune cell that naturally occurs in the immune system.

As used herein, the term “increase” means to alter positively by at least about 5%, including, but not limited to, alter positively by about 5%, by about 10%, by about 25%, by about 30%, by about 50%, by about 75%, or by about 100%.

As used herein, the terms “individual”, “patient”, or “subject” can be an individual organism, a vertebrate, a mammal, or a human. In some embodiments, the individual, patient or subject is a human.

As used herein, the term “MHC” refers to the Major Histocompability Complex, which is defined as a set of gene loci specifying major histocompatibility antigens. The term “HLA” as used herein will be understood to refer to Human Leukocyte Antigens, which is defined as the histocompatibility antigens found in humans. As used herein, “HLA” is the human form of “MHC”.

As used herein, the terms “MHC light chain” and “MHC heavy chain” refer to portions of the MHC molecule. Structurally, class I molecules are heterodimers comprised of two noncovalently bound polypeptide chains, a larger “heavy” chain (a) and a smaller “light” chain (β₂-microglobulin or β₂m). The polymorphic, polygenic heavy chain (45 kDa), encoded within the MHC on chromosome six, is subdivided into three extracellular domains (designated 1, 2, and 3), one intracellular domain, and one transmembrane domain. The two outermost extracellular domains, 1 and 2, together form the groove that binds antigenic peptide. Thus, interaction with the TCR occurs at this region of the protein. The 3 domain of the molecule contains the recognition site for the CD8 protein on the CTL; this interaction serves to stabilize the contact between the T cell and the APC. The invariant light chain (12 kDa), encoded outside the MHC on chromosome 15, consists of a single, extracellular polypeptide. The terms “MHC light chain”, “β-2-microglobulin”, and “β₂m” may be used interchangeably herein.

The term “monoclonal antibody” as used herein refers to an antibody obtained from a population of substantially homogeneous antibodies, i.e., the individual antibodies comprising the population are identical except for possible naturally occurring mutations that may be present in minor amounts. For example, a monoclonal antibody can be an antibody that is derived from a single clone, including any eukaryotic, prokaryotic, or phage clone, and not the method by which it is produced. A monoclonal antibody composition displays a single binding specificity and affinity for a particular epitope. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. Monoclonal antibodies can be prepared using a wide variety of techniques known in the art including, e.g., but not limited to, hybridoma, recombinant, and phage display technologies. For example, the monoclonal antibodies to be used in accordance with the present methods may be made by the hybridoma method first described by Kohler et al., Nature 256:495 (1975), or may be made by recombinant DNA methods (See, e.g., U.S. Pat. No. 4,816,567). The “monoclonal antibodies” may also be isolated from phage antibody libraries using the techniques described in Clackson et al., Nature 352:624-628 (1991) and Marks et al., J. Mol. Biol. 222:581-597 (1991), for example.

As used herein, “operably linked” with reference to nucleic acid sequences, regions, elements or domains means that the nucleic acid regions are functionally related to each other. For example, a nucleic acid encoding a leader peptide can be operably linked to a nucleic acid encoding a polypeptide, whereby the nucleic acids can be transcribed and translated to express a functional fusion protein, wherein the leader peptide affects secretion of the fusion polypeptide. In some instances, the nucleic acid encoding a first polypeptide (e.g., a leader peptide) is operably linked to nucleic acid encoding a second polypeptide and the nucleic acids are transcribed as a single mRNA transcript, but translation of the mRNA transcript can result in one of two polypeptides being expressed. For example, an amber stop codon can be located between the nucleic acid encoding the first polypeptide and the nucleic acid encoding the second polypeptide, such that, when introduced into a partial amber suppressor cell, the resulting single mRNA transcript can be translated to produce either a fusion protein containing the first and second polypeptides, or can be translated to produce only the first polypeptide. In another example, a promoter can be operably linked to nucleic acid encoding a polypeptide, whereby the promoter regulates or mediates the transcription of the nucleic acid.

As used herein, the term “pharmaceutically-acceptable carrier” is intended to include any and all solvents, dispersion media, coatings, antibacterial and antifungal compounds, isotonic and absorption delaying compounds, and the like, compatible with pharmaceutical administration. Pharmaceutically-acceptable carriers and their formulations are known to one skilled in the art and are described, for example, in Remington's Pharmaceutical Sciences (20th edition, ed. A. Gennaro, 2000, Lippincott, Williams & Wilkins, Philadelphia, Pa.).

As used herein, the term “polyclonal antibody” means a preparation of antibodies derived from at least two (2) different antibody-producing cell lines. The use of this term includes preparations of at least two (2) antibodies that contain antibodies that specifically bind to different epitopes or regions of an antigen.

As used herein, the term “polynucleotide” or “nucleic acid” means any RNA or DNA, which may be unmodified or modified RNA or DNA. Polynucleotides include, without limitation, single- and double-stranded DNA, DNA that is a mixture of single- and double-stranded regions, single- and double-stranded RNA, RNA that is mixture of single- and double-stranded regions, and hybrid molecules comprising DNA and RNA that may be single-stranded or, more typically, double-stranded or a mixture of single- and double-stranded regions. In addition, polynucleotide refers to triple-stranded regions comprising RNA or DNA or both RNA and DNA. The term polynucleotide also includes DNAs or RNAs containing one or more modified bases and DNAs or RNAs with backbones modified for stability or for other reasons.

As used herein, the terms “polypeptide,” “peptide” and “protein” are used interchangeably herein to mean a polymer comprising two or more amino acids joined to each other by peptide bonds or modified peptide bonds, i.e., peptide isosteres. Polypeptide refers to both short chains, commonly referred to as peptides, glycopeptides or oligomers, and to longer chains, generally referred to as proteins. Polypeptides may contain amino acids other than the 20 gene-encoded amino acids. Polypeptides include amino acid sequences modified either by natural processes, such as post-translational processing, or by chemical modification techniques that are well known in the art. Such modifications are well described in basic texts and in more detailed monographs, as well as in a voluminous research literature.

As used herein, the term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the material is derived from a cell so modified. Thus, for example, recombinant cells express genes that are not found within the native (non-recombinant) form of the cell or express native genes that are otherwise abnormally expressed, under expressed or not expressed at all.

As used herein, “regulatory region” of a nucleic acid molecule means a cis-acting nucleotide sequence that influences expression, positively or negatively, of an operably linked gene. Regulatory regions include sequences of nucleotides that confer inducible (i.e., require a substance or stimulus for increased transcription) expression of a gene. When an inducer is present or at increased concentration, gene expression can be increased.

Regulatory regions also include sequences that confer repression of gene expression (i.e., a substance or stimulus decreases transcription). When a repressor is present or at increased concentration, gene expression can be decreased. Regulatory regions are known to influence, modulate or control many in vivo biological activities including cell proliferation, cell growth and death, cell differentiation and immune modulation. Regulatory regions typically bind to one or more trans-acting proteins, which results in either increased or decreased transcription of the gene.

Particular examples of gene regulatory regions are promoters and enhancers. Promoters are sequences located around the transcription or translation start site, typically positioned 5′ of the translation start site. Promoters usually are located within 1 Kb of the translation start site, but can be located further away, for example, 2 Kb, 3 Kb, 4 Kb, 5 Kb or more, up to and including 10 Kb. Enhancers are known to influence gene expression when positioned 5′ or 3′ of the gene, or when positioned in or a part of an exon or an intron. Enhancers also can function at a significant distance from the gene, for example, at a distance from about 3 Kb, 5 Kb, 7 Kb, 10 Kb, 15 Kb or more. Regulatory regions also include, but are not limited to, in addition to promoter regions, sequences that facilitate translation, splicing signals for introns, maintenance of the correct reading frame of the gene to permit in-frame translation of mRNA and, stop codons, leader sequences and fusion partner sequences, internal ribosome binding site (IRES) elements for the creation of multigene, or polycistronic, messages, polyadenylation signals to provide proper polyadenylation of the transcript of a gene of interest and stop codons, and can be optionally included in an expression vector.

As used herein, the term “sample” refers to clinical samples obtained from a subject. In certain embodiments, a sample is obtained from a biological source (i.e., a “biological sample”), such as tissue, bodily fluid, or microorganisms collected from a subject. Sample sources include, but are not limited to, mucus, sputum, bronchial alveolar lavage (BAL), bronchial wash (BW), whole blood, bodily fluids, cerebrospinal fluid (CSF), urine, plasma, serum, or tissue.

As used herein, the term “separate” therapeutic use refers to an administration of at least two active ingredients at the same time or at substantially the same time by different routes.

As used herein, the term “sequential” therapeutic use refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this case.

As used herein, “specifically binds” refers to a molecule (e.g., an immunoglobulin-related composition) which recognizes and binds another molecule (e.g., an antigen), but that does not substantially recognize and bind other molecules. The terms “specific binding,” “specifically binds to,” or is “specific for” a particular molecule, as used herein, can be exhibited, for example, by a molecule having a KD for the molecule to which it binds to of about 10⁻⁴ M, 10⁻⁵M, 10⁻⁶M, 10⁻⁷M, 10⁻⁸ M, 10⁻⁹ M, 10⁻¹⁰ M, 10⁻¹¹ M, or 10⁻¹² M. The term “specifically binds” may also refer to binding where a molecule (e.g., TCR) binds to a particular target molecule or complex (e.g., peptides presented on cell surfaces in the context of major histocompability complexes), without substantially binding to any other molecule or complex.

As used herein, the term “simultaneous” therapeutic use refers to the administration of at least two active ingredients by the same route and at the same time or at substantially the same time.

As used herein, “synthetic,” with reference to, for example, a synthetic nucleic acid molecule or a synthetic gene or a synthetic peptide refers to a nucleic acid molecule or polypeptide molecule that is produced by recombinant methods and/or by chemical synthesis methods. As used herein, “production by recombinant means by using recombinant DNA methods” means the use of the well-known methods of molecular biology for expressing proteins encoded by cloned DNA.

As used herein, the term “T-cell” includes naïve T cells, CD4+ T cells, CD8+ T cells, memory T cells (including central memory T cells, stem-cell-like memory T cells (or stem-like memory T cells), and two types of effector memory T cells: e.g., TEM cells and TEMRA cells), activated T cells, anergic T cells, tolerant T cells, chimeric B cells, Regulatory T cells (also known as suppressor T cells), Natural killer T cells, Mucosal associated invariant T cells, and γδ T cells, and antigen-specific T cells.

As used herein, “T cell receptor” or “TCR”, is a protein complex found on the surface of T cells that is responsible for recognizing fragments of antigen as peptides bound to major histocompatibility complex molecules. TCR is composed of two disulfide-linked protein chains. Cells expressing a TCR containing the highly variable alpha (a) and beta (B) chains are referred to as αβ T cells. Cells expressing an alternate TCR, formed by variable gamma (γ) and delta (δ) chains, are referred to as γδ T cells. When the TCR engages with antigenic peptide and MHC (peptide/MHC or pMHC), the T lymphocyte is activated through signal transduction, that is, a series of biochemical events mediated by associated enzymes, co-receptors, specialized adaptor molecules, and activated or released transcription factors. In some embodiments, a TCR is a native T cell receptor that is endogenous to the immune cells. In some embodiments, a TCR is an artificial receptor that mimics native TCR function, i.e., recognizing peptide antigens of key intracellular proteins in the context of MHC on the cell surface.

The term “TCR-associated signaling molecule” refers to a molecule having a cytoplasmic immunoreceptor tyrosine-based activation motif (ITAM) that is part of the TCR-CD3 complex. TCR-associated signaling molecules include CD3γε, CD3δε, and CD3ζ (also known as ζζ, CD3ζζ, or TCRζ).

As used herein, the terms “mimic TCR,” “TCRm” or “TCR-like” refers to an artificial receptor that mimics native TCR function (i.e., recognizing peptide antigens of key intracellular proteins in the context of MHC on the cell surface). Examples of different types mimic TCRs (e.g., TCRm Abs, TCRm BITEs, TCRm CARs, and ImmTACs) are described in detail in Jones et al., Front. Immunol., 25 Jan. 2021, doi.org/10.3389/fimmu.2020.585385, the contents of which are incorporated by reference herein in its entirety.

As used herein, the term “therapeutic agent” is intended to mean a compound that, when present in an effective amount, produces a desired therapeutic effect on a subject in need thereof.

“Treating” or “treatment” as used herein covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. In some embodiments, treatment means that the symptoms associated with the disease are, e.g., alleviated, reduced, cured, or placed in a state of remission.

It is also to be appreciated that the various modes of treatment of disorders as described herein are intended to mean “substantial,” which includes total but also less than total treatment, and wherein some biologically or medically relevant result is achieved. The treatment may be a continuous prolonged treatment for a chronic disease or a single, or few time administrations for the treatment of an acute condition.

As used herein, a “vector” is a replicable nucleic acid from which one or more heterologous proteins can be expressed when the vector is transformed into an appropriate host cell. Reference to a vector includes those vectors into which a nucleic acid encoding a polypeptide or fragment thereof can be introduced, typically by restriction digest and ligation. Reference to a vector also includes those vectors that contain nucleic acid encoding a polypeptide. The vector is used to introduce the nucleic acid encoding the polypeptide into the host cell for amplification of the nucleic acid or for expression/display of the polypeptide encoded by the nucleic acid. The vectors typically remain episomal, but can be designed to effect integration of a gene or portion thereof into a chromosome of the genome. Also contemplated are vectors that are artificial chromosomes, such as yeast artificial chromosomes and mammalian artificial chromosomes. Selection and use of such vehicles are well known to those of skill in the art. A vector also includes “virus vectors” or “viral vectors.” Viral vectors are engineered viruses that are operably linked to exogenous genes to transfer (as vehicles or shuttles) the exogenous genes into cells. As used herein, an “expression vector” includes vectors capable of expressing DNA that is operably linked with regulatory sequences, such as promoter regions, that are capable of effecting expression of such DNA fragments. Such additional segments can include promoter and terminator sequences, and optionally can include one or more origins of replication, one or more selectable markers, an enhancer, a polyadenylation signal, and the like. Expression vectors are generally derived from plasmid or viral DNA, or can contain elements of both. Thus, an expression vector refers to a recombinant DNA or RNA construct, such as a plasmid, a phage, recombinant virus or other vector that, upon introduction into an appropriate host cell, results in expression of the cloned DNA. Appropriate expression vectors are well known to those of skill in the art and include those that are replicable in eukaryotic cells and/or prokaryotic cells and those that remain episomal or those which integrate into the host cell genome.

### T-Cell Receptors (TCRs)

T cells are part of the adaptive immune system and target cancerous and/or infected cells through T-cell receptors (TCRs). T cell receptors are protein complexes found on the surface of T cells and are responsible for recognizing fragments of antigen as peptides bound to major histocompatibility complex molecules. TCRs are composed of two disulfide-linked protein chains. Cells expressing a TCR containing the highly variable alpha (α) and beta (β) chains are referred to as αβ T cells. Cells expressing an alternate TCR, formed by variable gamma (γ) and delta (δ) chains, are referred to as γδ T cells. Without wishing to be bound by any one theory, variable chains allows for the recognition of many different peptides (e.g., target antigens, including, for example, tumor antigens) presented on MHC molecules.

There are two major groups of MHC molecules, class I (MHC I) and class II (MHC II). MHC I molecules interact with CD8+ T cells and MHC II molecules interact with CD4+T cells. MHC I molecules present peptides, typically 8-12 amino acids long, derived from proteins in any cellular compartment. Presented peptides (e.g., target antigens, including, for example, tumor antigens) are generated as a result of protein degradation through the proteasome, cleavage by aminopeptidases, and transport to the Endoplasmic Reticulum (ER) via the transporter associated with antigen processing (TAP). Peptides are then loaded onto MHC I molecules, resulting in a peptide-MHC complex. These peptide-MHC (pMHC) complexes are then shuttled to the cell surface where they are presented to CD8+ T cells and are recognized by TCRs. When TCRs engage with antigenic peptide and MHC (peptide/MHC), the T cell is activated through signal transduction, that is, a series of biochemical events mediated by associated enzymes, co-receptors, specialized adaptor molecules, and activated or released transcription factors, ultimately resulting in immune-mediated cell death.

Accordingly, in some embodiments, a TCR as disclosed herein binds to a target antigen. In further embodiments, a target antigen is a tumor antigen presented in the context of a MHC I molecule. In some such embodiments, a tumor antigen is or comprises, for example, Tyrosinase, NY-ESO-1, CD277-mediated presentation, MAGE-A4, WT1, MAGE-A10, PRAME, EBV LMP2, MAGE-A1, HA-1, HERV-E, CMV pp65, HBV, TRAIL-DR4, HIV SL9, or AFP. In some embodiments, a MHC I molecule is a HLA-A, HLA-B, or HLA-C molecule. In some embodiments, a target antigen comprises a tumor antigen presented in the context of a HLA-A2 molecule.

In some embodiments, a TCR binds to a target antigen that is expressed by a tumor cell (e.g., a tumor antigen). In some embodiments, a TCR binds to a target antigen that is expressed on the surface of a tumor cell as part of the pMHC complex. In some such embodiments, a target antigen expressed on the surface of a tumor cell as part of the pMHC complex is expressed intracellularly when not as part of the pMHC complex. In some embodiments, the MHC protein is a MHC class I protein. In some embodiments, the MHC class I protein is a HLA-A, HLA-B, or HLA-C molecule. In some embodiments, a HLA-A molecule is a HLA-A2 molecule.

In some embodiments, a TCR is a native T cell receptor that is endogenous to an immune cell (e.g., T cell). In some such embodiments, a native TCR is or comprises, for example, 1G4-α95TS (also referred to as 1G4a95TS, native 1G4 TCR, and native 1G4; see, e.g., Robbins P F et al., Single and dual amino acid substitutions in TCR CDRs can enhance antigen-specific T cell functions. J Immunol. 2008; 180(9):6116-6131. doi:10.4049/jimmunol.180.9.6116).

In some embodiments, a TCR is a non-native TCR. In some such embodiments, a non-native TCR is an engineered TCR that binds to a target antigen (e.g., tumor antigen). In some embodiments, an engineered TCR is an affinity enhanced TCR. In some such embodiments, an affinity enhanced TCR is or comprises, for example, 1G4 TCR (also referred to as 1G4-α95LY and 1G4a95LY; see, e.g., Robbins P F et al., Single and dual amino acid substitutions in TCR CDRs can enhance antigen-specific T cell functions. J Immunol. 2008; 180(9):6116-6131. doi:10.4049/jimmunol.180.9.6116).

In some embodiments, a TCR is a mimic TCR. A mimic TCR is, for example, an artificial receptor that mimics native TCR function (i.e., recognizing peptide antigens of key intracellular proteins in the context of MHC on the cell surface).

In some embodiments, a TCR is 1G4, DMF5, or A6 TCR. In some embodiments, a TCR is an affinity enhanced IG4, DMF5, or A6 TCR.

In some embodiments, a TCR comprises a high binding specificity and/or high binding affinity to a target antigen. For example, in some embodiments, a TCR binds to a particular target antigen with a dissociation constant (K_{d}) of about 1×10⁻⁵ M or less. In certain embodiments, the K_{d} is about 5×10⁻⁶ M or less, about 1×10⁻⁶ M or less, about 5×10⁻⁷ M or less, about 1×10 7 M or less, about 5×10⁻⁸ M or less, about 1×10⁻⁸ M or less, about 5×10⁻⁹ or less, about 4×10⁻⁹ or less, about 3×10⁻⁹ or less, about 2×10⁻⁹ or less, or about 1×10⁻⁹ M or less. In certain non-limiting embodiments, the K_{d} is from about 3×10⁻⁹ M or less. In certain non-limiting embodiments, the K_{d} is from about 3×10⁻⁹ to about 2×10⁻⁷.

Binding of a TCR of the present disclosure can be assessed by, for example, enzyme-linked immunosorbent assay (ELISA), enzyme-linked immunosorbent spot (ELISpot) assay, radioimmunoassay (RIA), FACS analysis, bioassay (e.g., growth inhibition), or Western Blot assay. Each of these assays generally detect the presence of protein-antibody complexes of particular interest by employing a labeled reagent (e.g., an antibody, or an scFv) specific for the complex of interest. For example, the scFv can be radioactively labeled and used in a radioimmunoassay (RIA) (see, for example, Weintraub, B., Principles of Radioimmunoassays, Seventh Training Course on Radioligand Assay Techniques, The Endocrine Society, March 1986, which is incorporated by reference herein). The radioactive isotope can be detected by such means as the use of a γ counter or a scintillation counter or by autoradiography. In certain embodiments, a TCR is labeled with a fluorescent marker. Non-limiting examples of fluorescent markers include green fluorescent protein (GFP), blue fluorescent protein (e.g., EBFP, EBFP2, Azurite, and mKalamal), cyan fluorescent protein (e.g., ECFP, Cerulean, and CyPet), and yellow fluorescent protein (e.g., YFP, Citrine, Venus, and YPet).

The presently disclosed subject matter also provides nucleic acids encoding TCRs as described herein or a functional portion thereof. In some embodiments, nucleic acids encoding TCRs are isolated nucleic acid molecules. In certain embodiments, a nucleic acid molecule comprises a nucleic acid sequence that encodes a functional portion of a TCR construct. As used herein, the term “functional portion” refers to any portion, part or fragment of a TCR, which portion, part, or fragment retains the biological activity of the parent TCR. For example, functional portions encompass the portions, parts, or fragments of a TCR that retains the ability to recognize peptides presented on the cell surface in the context of a MHC (e.g., MHC I) to a similar, same, or even higher extent as the parent TCR. In certain embodiments, a nucleic acid molecule encoding a functional portion of a target-antigen specific TCR can encode a protein comprising, about 10%, about 20%, about 25%, about 30%, about 35%, about 40%, about 45%, about 50%, about 55%, about 60%, about 65%, about 70%, about 75%, about 80%, about 85%, about 90%, and about 95%, or more of the parent TCR.

Additionally or alternatively, in certain embodiments, a nucleic acid comprises a nucleic acid sequence encoding a TCR and a first reporter or selection marker (e.g., GFP, puromycin resistance). In some such embodiments, the nucleic acid encoding the TCR and first reporter or selection marker are separated by a nucleic acid sequence encoding an Internal Ribosomal Entry Site (IRES). In some such embodiments, the TCR and first reporter or selection marker are linked by a self-cleaving linker, such as a P2A linker. In certain embodiments, a heterologous TCR and a reporter or selection marker (e.g., GFP, puromycin resistance) are expressed as two separate polypeptides.

Additionally or alternatively, in certain embodiments, a nucleic acid comprises a nucleic acid sequence encoding a TCR and a CD8α polypeptide (as described elsewhere herein). In some such embodiments, the TCR and a CD8α polypeptide are linked by a self-cleaving linker, such as a P2A linker. In certain embodiments, a heterologous TCR and a CD8α polypeptide are expressed as two separate polypeptides. In some such embodiments, the nucleic acid sequence encoding a TCR and a CD8α polypeptide are separated by a nucleic acid sequence encoding an Internal Ribosomal Entry Site (IRES).

Additionally or alternatively, in some embodiments, a heterologous nucleic acid comprising a nucleic acid sequence encoding a CD8α polypeptide (as described elsewhere herein) and/or a TCR of the present disclosure is operably linked to an inducible promoter. In some embodiments, a heterologous nucleic acid comprising a nucleic acid sequence encoding a CD8α gene (as described elsewhere herein) and/or a TCR of the present disclosure is operably linked to a constitutive promoter.

Among other things, technologies of the present disclosure provide engineered immune cells (e.g., engineered cytotoxic T cells, engineered CD4+ helper T cells) that comprise a TCR that binds to a target antigen and/or a nucleic acid encoding the TCR. In some embodiments, engineered immune cells of the present disclosure express CD8α (as described elsewhere herein) and/or a TCR (e.g., a native TCR, a non-native TCR, mimic TCR). In certain embodiments, engineered immune cells can be transduced with a TCR construct such that the cells express the TCR. In some embodiments, a coding sequence of a TCR is endogenously present in an engineered immune cell. In some embodiments, a coding sequence of a TCR is exogenously provided to an engineered immune cell via a nucleic acid vector, such as a retroviral vector. Technologies of the present disclosure also provide, among other things, methods of using such cells for the treatment of cancer.

### CD8α

As used herein, the terms “CD8 alpha”, “CD8α”, “CD8α polypeptide” “Leu2”, and “P32” refer to a cell surface glycoprotein found on most cytotoxic T cells that mediates efficient cell-cell interactions within the immune system. The encoded protein also acts as a co-receptor with TCRs on the T cell to recognize antigens displayed by an antigen presenting cell in the context of class I MHC molecules. The CD8 co-receptor functions as either a homodimer, comprised of two alpha (α) chains, or as a heterodimer, comprised of one alpha (α) and one beta (β) chain. Non-limiting examples of this polypeptide or underlying gene may be found under the Gene Cards IDs: GC02M086907 GC02M087305, GC02M086986, GC02M086923, GC02M086865 (retrieved from https://www.genecards.org/cgi-bin/carddisp.pl?gene-CD8A #summaries), HGNC:1706 (https://www.genenames.org/data/gene-symbol-report/#!/hgnc_id/1706), NCBI Entrez Gene: 925 (https://www.ncbi.nlm.nih.gov/gene/925), Ensembl: ENSG00000153563 (https://useast.ensembl.org/Homo_sapiens/Gene/Summary?g=ENSG00000153563;r=2:86784 610-86808396), OMIM®: 186910 (https://omim.org/entry/186910), or UniProtKB/Swiss-Prot: P01732 (https://www.uniprot.org/uniprot/P01732), which are incorporated by reference herein.

The presently disclosed subject matter also provides nucleic acids encoding CD8α polypeptides as described herein or a functional portion thereof. As used herein, the term “functional portion” refers to any portion, part or fragment of a CD8α polypeptide, which portion, part, or fragment retains the biological activity of the parent CD8α polypeptide. For example, functional portions encompass the portions, parts, or fragments of a CD8α polypeptide that retains the ability to bind to MHC relative to the wild-type CD8α polypeptide to a similar, same, or even higher extent as the parent CD8α polypeptide. In certain embodiments, a nucleic acid molecule encoding a functional portion of a CD8α polypeptide can encode a polypeptide comprising, about 10%, about 20%, about 25%, about 30%, about 35%, about 40%, about 45%, about 50%, about 55%, about 60%, about 65%, about 70%, about 75%, about 80%, about 85%, about 90%, and about 95%, or more of the parent CD8α polypeptide. In some embodiments, nucleic acids encoding CD8α polypeptides are isolated nucleic acids. In some embodiments, nucleic acids encoding CD8α polypeptide encode a mutant CD8α polypeptide. Exemplary nucleotide sequences of CD8α polypeptides are set forth in Table 1. Accordingly, in some embodiments, a nucleic acid sequence encoding a wild-type (WT) CD8α polypeptide is or comprises the nucleic acid sequence of SEQ ID NO: 33. In some embodiments, a nucleic acid sequence encoding a mutant (M2) CD8α polypeptide is or comprises the nucleic acid sequence of SEQ ID NO: 34. In some embodiments, a nucleic acid sequence encoding a mutant (M5) CD8α polypeptide is or comprises the nucleic acid sequence of SEQ ID NO: 35.

Nucleic acids of the present disclosure encode, for example, WT or mutant CD8α polypeptides. In some embodiments, mutant CD8α polypeptides exhibit reduced activity (e.g., reduced binding to MHC) relative to the wild-type CD8α polypeptide. In some embodiments, mutant CD8α polypeptides exhibit increased activity (e.g., increased binding to MHC) relative to the wild-type CD8α polypeptide. Exemplary amino acid sequences of CD8α are set forth in Table 2. Accordingly, in some embodiments, a wild-type (WT) CD8α polypeptide is or comprises the amino acid sequence of SEQ ID NO: 36 or SEQ ID NO: 47. In some embodiments, a mutant (M2) CD8α polypeptide is or comprises the amino acid sequence of SEQ ID NO: 37 or SEQ ID NO: 48. In some embodiments, a mutant (M5) CD8α polypeptide is or comprises the amino acid sequence of SEQ ID NO: 38 or SEQ ID NO: 49.

Additionally or alternatively, in certain embodiments, a nucleic acid comprises a nucleic acid sequence encoding a WT or mutant CD8α polypeptide (e.g., M2 CD8α, M5 CD8α) and a first reporter or selection marker (e.g., GFP, puromycin resistance). In some such embodiments, the WT or mutant CD8α polypeptide and the first reporter or selection marker are linked by a self-cleaving linker, such as a P2A linker. In certain embodiments, a heterologous WT or mutant CD8α polypeptide and a reporter or selection marker (e.g., GFP, puromycin resistance) are expressed as two separate polypeptides. In some such embodiments, the nucleic acid encoding a WT or mutant CD8α polypeptide and the first reporter or selection marker are separated by a nucleic acid sequence encoding an Internal Ribosomal Entry Site (IRES).

Additionally or alternatively, in certain embodiments, a nucleic acid comprises a nucleic acid sequence encoding a WT or mutant CD8α polypeptide (e.g., M2 CD8α, M5 CD8α) and a TCR (as described elsewhere herein). In some such embodiments, the CD8α and TCR are linked by a self-cleaving linker, such as a P2A linker. In certain embodiments, a heterologous CD8α and a TCR are expressed as two separate polypeptides. In some such embodiments, the nucleic acid sequence encoding a CD8α and a TCR are separated by a nucleic acid sequence encoding an Internal Ribosomal Entry Site (IRES).

Additionally or alternatively, in some embodiments, a heterologous nucleic acid comprising a nucleic acid sequence encoding a CD8α (e.g., M2 CD8α, M5 CD8α) and/or a TCR of the present disclosure is operably linked to an inducible promoter. In some embodiments, a heterologous nucleic acid comprising a nucleic acid sequence encoding a CD8α gene (e.g., M2 CD8α, M5 CD8α) and/or a TCR of the present disclosure is operably linked to a constitutive promoter.

Among other things, technologies of the present disclosure provide engineered immune cells (e.g., engineered cytotoxic T cells, engineered CD4+ helper T cells) that comprise a non-endogenous expression vector that includes a nucleic acid sequence encoding a WT or mutant CD8α polypeptide, e.g., M2 CD8α, M5 CD8α. In some embodiments, engineered immune cells of the present disclosure express WT or mutant CD8α and a TCR (e.g., a native TCR, a non-native TCR, a mimic TCR). In certain embodiments, engineered immune cells can be transduced with a nucleic acid sequence encoding a CD8α polypeptide (e.g., WT or mutant CD8α) polypeptide. In some embodiments, a coding sequence of a CD8α polypeptide is endogenously present in an engineered immune cell (e.g., engineered cytotoxic T cells). In some embodiments, a coding sequence of a WT or mutant CD8α is exogenously provided to an engineered immune cell via a nucleic acid vector, such as a retroviral vector. Technologies of the present disclosure also provide, among other things, methods of using such cells for the treatment of cancer.

### Vectors

Many expression vectors are available and known to those of skill in the art and can be used for expression of polypeptides (e.g., TCRs, CD8α polypeptides) provided herein. The choice of expression vector will be influenced by the choice of host expression system. Such selection is well within the level of skill of the skilled artisan. In general, expression vectors can include transcriptional promoters and optionally enhancers, translational signals, and transcriptional and translational termination signals. Expression vectors that are used for stable transformation typically have a selectable marker which allows selection and maintenance of the transformed cells. Selectable markers include, for example, fluorescent markers (e.g., green fluorescent protein, mCherry, etc.) or antibiotic resistance markers (e.g., puromycin resistance, ampicillin resistance, etc.) In some cases, an origin of replication can be used to amplify the copy number of the vector in the cells.

Vectors also can contain additional nucleotide sequences operably linked to the ligated nucleic acid molecule, such as, for example, an epitope tag such as for localization (e.g., signal sequences, including, for example, ER signaling sequences), e.g., a hexa-his tag (SEQ ID NO: 50) or a myc tag (e.g., EQKLISEEDL (SEQ ID NO: 12)), hemagglutinin tag or a tag for purification, for example, a GST fusion, and a sequence for directing protein secretion and/or membrane association.

Expression of polypeptides, including, for example, T cell Receptors and CD8α polypeptides, can be controlled by any promoter/enhancer known in the art. In some embodiments, a promoter is an inducible promoter, a constitutive promoter, a native promoter (e.g., CD8 or CD4 promoter), or a heterologous promoter. Suitable bacterial promoters are well known in the art and described herein below. Other suitable promoters for mammalian cells, yeast cells and insect cells are well known in the art and some are exemplified below. Selection of the promoter used to direct expression of a heterologous nucleic acid depends on the particular application and is within the level of skill of the skilled artisan. Promoters which can be used include but are not limited to eukaryotic expression vectors containing the SV40 early promoter (Bernoist and Chambon, Nature 290:304-310 (1981)), the promoter contained in the 3′ long terminal repeat of Rous sarcoma virus (Yamamoto et al., Cell 22:787-797(1980)), the herpes thymidine kinase promoter (Wagner et al., Proc. Natl. Acad. Sci. USA 75:1441-1445 (1981)), the regulatory sequences of the metallothionein gene (Brinster et al., Nature 296:39-42 (1982)); prokaryotic expression vectors such as the β-lactamase promoter (Jay et al., Proc. Natl. Acad. Sci. USA 75:5543 (1981)) or the tac promoter (DeBoer et al., Proc. Natl. Acad. Sci. USA 50:21-25(1983)); see also “Useful Proteins from Recombinant Bacteria”: in Scientific American 242:79-94 (1980)); plant expression vectors containing the nopaline synthetase promoter (Herrera-Estrella et al., Nature 505:209-213(1984)) or the cauliflower mosaic virus 35S RNA promoter (Gardner et al., Nucleic Acids Res. 9:2871(1981)), and the promoter of the photosynthetic enzyme ribulose bisphosphate carboxylase (Herrera-Estrella et al., Nature 510:1 15-120(1984)); promoter elements from yeast and other fungi such as the Gal4 promoter, the alcohol dehydrogenase promoter, the phosphoglycerol kinase promoter, the alkaline phosphatase promoter, and the following animal transcriptional control regions that exhibit tissue specificity and have been used in transgenic animals: elastase I gene control region which is active in pancreatic acinar cells (Swift et al., Cell 55:639-646 (1984); Ornitz et al., Cold Spring Harbor Symp. Quant. Biol. 50:399-409(1986); MacDonald, Hepatology 7:425-515 (1987)); insulin gene control region which is active in pancreatic beta cells (Hanahan et al., Nature 515:115-122 (1985)), immunoglobulin gene control region which is active in lymphoid cells (Grosschedl et al., Cell 55:647-658 (1984); Adams et al., Nature 515:533-538 (1985); Alexander et al., Mol. Cell Biol. 7:1436-1444 (1987)), mouse mammary tumor virus control region which is active in testicular, breast, lymphoid and mast cells (Leder et al., Cell 15:485-495 (1986)), albumin gene control region which is active in liver (Pinckert et al., Genes and Devel. 1:268-276 (1987)), alpha-fetoprotein gene control region which is active in liver (Krumlauf et al., Mol. Cell. Biol. 5:1639-403 (1985)); Hammer et al., Science 255:53-58 (1987)), alpha-1 antitrypsin gene control region which is active in liver (Kelsey et al., Genes and Devel. 7:161-171 (1987)), beta globin gene control region which is active in myeloid cells (Magram et al., Nature 515:338-340 (1985)); Kollias et al., Cell 5:89-94 (1986)), myelin basic protein gene control region which is active in oligodendrocyte cells of the brain (Readhead et al., Cell 15:703-712 (1987)), myosin light chain-2 gene control region which is active in skeletal muscle (Shani, Nature 514:283-286 (1985)), and gonadotrophic releasing hormone gene control region which is active in gonadotrophs of the hypothalamus (Mason et al., Science 254:1372-1378 (1986)).

In addition to the promoter, the expression vector typically contains a transcription unit or expression cassette that contains all the additional elements required for the expression of an antibody, or antigen binding fragment thereof, in host cells. A typical expression cassette contains a promoter operably linked to the nucleic acid sequence encoding the polypeptide chains of interest and signals required for efficient polyadenylation of the transcript, ribosome binding sites and translation termination. Additional elements of the cassette can include enhancers. In addition, the cassette typically contains a transcription termination region downstream of the structural gene to provide for efficient termination. The termination region can be obtained from the same gene as the promoter sequence or can be obtained from different genes.

Some expression systems have markers that provide gene amplification such as thymidine kinase and dihydrofolate reductase. Alternatively, high yield expression systems not involving gene amplification are also suitable, such as using a baculovirus vector in insect cells, with a nucleic acid sequence encoding a germline antibody chain under the direction of the polyhedron promoter or other strong baculovirus promoter.

Any methods known to those of skill in the art for the insertion of DNA fragments into a vector can be used to construct expression vectors containing a nucleic acid encoding any of the polypeptides provided herein. These methods can include in vitro recombinant DNA and synthetic techniques and in vivo recombinants (genetic recombination). The insertion into a cloning vector can, for example, be accomplished by ligating the DNA fragment into a cloning vector which has complementary cohesive termini. If the complementary restriction sites used to fragment the DNA are not present in the cloning vector, the ends of the DNA molecules can be enzymatically modified. Alternatively, any site desired can be produced by ligating nucleotide sequences (linkers) onto the DNA termini; these ligated linkers can contain specific chemically synthesized nucleic acids encoding restriction endonuclease recognition sequences.

Exemplary plasmid vectors useful to produce the polypeptides provided herein contain a strong promoter, such as the HCMV immediate early enhancer/promoter or the MHC class I promoter, an intron to enhance processing of the transcript, such as the HCMV immediate early gene intron A, and a polyadenylation (poly A) signal, such as the late SV40 polyA signal.

Genetic modification of engineered immune cells (e.g., T cells) can be accomplished by transducing a substantially homogeneous cell composition with a recombinant DNA or RNA construct. The vector can be a retroviral vector (e.g., gamma retroviral), which is employed for the introduction of the DNA or RNA construct into the host cell genome. For example, a polynucleotide encoding a certain TCR (e.g., 1G4 TCR) and/or a certain CD8α polypeptides (e.g., CD8α WT, CD8α M2, CD8α M5) can be cloned into a retroviral vector and expression can be driven from its endogenous promoter, from the retroviral long terminal repeat, or from an alternative internal promoter.

Non-viral vectors or RNA may be used as well. Random chromosomal integration, or targeted integration (e.g., using a nuclease, transcription activator-like effector nucleases (TALENs), Zinc-finger nucleases (ZFNs), and/or clustered regularly interspaced short palindromic repeats (CRISPRs), or transgene expression (e.g., using a natural or chemically modified RNA) can be used.

For initial genetic modification of the cells to provide certain TCR (e.g., 1G4 TCR) and/or CD8α polypeptides (e.g., CD8α WT, CD8α M2, CD8α M5), a retroviral vector is generally employed for transduction, however any other suitable viral vector or non-viral delivery system can be used. In some embodiments, for example, non-endogenous expression vectors are utilized. Non-limiting examples of non-endogenous expression vectors include a plasmid, a cosmid, a bacmid, a bacterial artificial chromosome (BAC), a yeast artificial chromosome (YAC), a viral vector, or a retroviral vector. For subsequent genetic modification of the cells to provide cells comprising an antigen presenting complex comprising at least two co-stimulatory ligands, retroviral gene transfer (transduction) likewise proves effective. Combinations of retroviral vector and an appropriate packaging line are also suitable, where the capsid proteins will be functional for infecting human cells.

Various amphotropic virus-producing cell lines are known, including, but not limited to, PA12 (Miller, et al., Mol. Cell. Biol. 5:431-437 (1985)); PA317 (Miller, et al., Mol. Cell. Biol. 6:2895-2902 (1986)); and CRIP (Danos, et al. Proc. Natl. Acad. Sci. USA 85:6460-6464 (1988)). Non-amphotropic particles are suitable too, e.g., particles pseudotyped with VSVG, RD114 or GALV envelope and any other known in the art.

Possible methods of transduction also include direct co-culture of the cells with producer cells, e.g., by the method of Bregni, et al., Blood 80:1418-1422 (1992), or culturing with viral supernatant alone or concentrated vector stocks with or without appropriate growth factors and polycations, e.g., by the method of Xu, et al., Exp. Hemat. 22:223-230 (1994); and Hughes, et al., J. Clin. Invest. 89:1817 (1992).

Transducing viral vectors can be used to express a co-stimulatory ligand and/or secrete a cytokine (e.g., 4-1BBL and/or IL-12) in an engineered immune cell. In some embodiments, the chosen vector exhibits high efficiency of infection and stable integration and expression (see, e.g., Cayouette et al., Human Gene Therapy 8:423-430 (1997); Kido et al., Current Eye Research 15:833-844 (1996); Bloomer et al., Journal of Virology 71:6641-6649, 1997; Naldini et al., Science 272:263 267 (1996); and Miyoshi et al., Proc. Natl. Acad. Sci. U.S.A. 94:10319, (1997)). Other viral vectors that can be used include, for example, adenoviral, lentiviral, and adeno-associated viral vectors, vaccinia virus, a bovine papilloma virus, or a herpes virus, such as Epstein-Barr Virus (also see, for example, the vectors of Miller, Human Gene Therapy 15-14, (1990); Friedman, Science 244:1275-1281 (1989); Eglitis et al., BioTechniques 6:608-614, (1988); Tolstoshev et al., Current Opinion in Biotechnology 1:55-61(1990); Sharp, The Lancet 337:1277-1278 (1991); Cornetta et al., Nucleic Acid Research and Molecular Biology 36:311-322 (1987); Anderson, Science 226:401-409 (1984); Moen, Blood Cells 17:407-416 (1991); Miller et al., Biotechnology 7:980-990 (1989); Le Gal La Salle et al., Science 259:988-990 (1993); and Johnson, Chest 107:77S-83S (1995)). Retroviral vectors are particularly well developed and have been used in clinical settings (Rosenberg et al., N. Engl. J. Med 323:370 (1990); Anderson et al., U.S. Pat. No. 5,399,346).

In certain non-limiting embodiments, the vector expressing a presently disclosed TCR (e.g., 1G4 TCR) and/or CD8α polypeptide (e.g., CD8α WT, CD8α M2, CD8α M5) is a retroviral vector, e.g., an oncoretroviral vector.

Non-viral approaches can also be employed for the expression of a protein in a cell. For example, a nucleic acid molecule can be introduced into a cell by administering the nucleic acid in the presence of lipofection (Feigner et al., Proc. Nat'l. Acad. Sci. U.S.A. 84:7413, (1987); Ono et al., Neuroscience Letters 17:259 (1990); Brigham et al., Am. J. Med. Sci. 298:278, (1989); Staubinger et al., Methods in Enzymology 101:512 (1983)), asialoorosomucoid-polylysine conjugation (Wu et al., Journal of Biological Chemistry 263:14621 (1988); Wu et al., Journal of Biological Chemistry 264:16985 (1989)), or by micro-injection under surgical conditions (Wolff et al., Science 247:1465 (1990)). Other non-viral means for gene transfer include transfection in vitro using calcium phosphate, DEAE dextran, electroporation, and protoplast fusion. Liposomes can also be potentially beneficial for delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically. Recombinant receptors can also be derived or obtained using transposases or targeted nucleases (e.g., Zinc finger nucleases, meganucleases, or TALE nucleases). Transient expression may be obtained by RNA electroporation.

cDNA expression for use in engineered immune cells, as described elsewhere herein, can be directed from any suitable promoter (e.g., the human cytomegalovirus (CMV), simian virus 40 (SV40), or metallothionein promoters), and regulated by any appropriate mammalian regulatory element or intron (e.g., the elongation factor 1a enhancer/promoter/intron structure). For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

The resulting cells can be grown under conditions similar to those for unmodified cells, whereby the modified cells can be expanded and used for a variety of purposes.

### Engineered Immune Cells of the Present Technology

Technologies of the present disclosure provide, among other things, engineered immune cells (e.g., engineered cytotoxic T cells, engineered CD4+ helper T cells) comprising a CD8α polypeptide (e.g., CD8α MWT, CD8α M2, or CD8α M5) and a T cell receptor that binds to a target antigen (e.g., 1G4 TCR). In certain embodiments, engineered immune cells can be transduced with a vector comprising a nucleic acid sequence that encodes a CD8α polypeptide (e.g., CD8α MWT, CD8α M2, or CD8α M5) and/or a vector comprising nucleic acid sequence that encodes a TCR that binds to a target antigen (e.g., 1G4 TCR).

The presently disclosed subject matter provides, among other things, engineered immune cells and methods of using such cells for the treatment of cancer. The engineered immune cells of the presently disclosed subject matter are T cells.

The lymphoid lineage, comprising B, T, and natural killer (NK) cells, provides for the production of antibodies, regulation of the cellular immune system, detection of foreign agents in the blood, detection of cells foreign to the host, and the like. Non-limiting examples of immune cells of the lymphoid lineage include T cells, Natural Killer (NK) cells, embryonic stem cells, and pluripotent stem cells (e.g., those from which lymphoid cells can be differentiated). T cells can be lymphocytes that mature in the thymus and are chiefly responsible for cell-mediated immunity. T cells are involved in the adaptive immune system. The T cells of the presently disclosed subject matter can be any type of T cells, including, but not limited to, T helper cells, cytotoxic T cells, memory T cells (including central memory T cells, stem-cell-like memory T cells (or stem-like memory T cells), and two types of effector memory T cells: e.g., TEM cells and TEMRA cells, Regulatory T cells (also known as suppressor T cells), Natural killer T cells, and Mucosal associated invariant T cells. Cytotoxic T cells (CTL or killer T cells) are a subset of T lymphocytes capable of inducing the death of infected somatic or tumor cells. In certain embodiments, T cells of the presently disclosed subject matter comprise engineered cytotoxic T cells or engineered CD4+ helper T cells.

In some embodiments, engineered immune cells of the present disclosure comprise an engineered cytotoxic T cell or an engineered CD4+ helper T cell that comprises a TCR that binds to a target antigen and/or a nucleic acid encoding the T cell receptor. In some embodiments, a TCR is a native TCR, a non-native TCR, or a mimic TCR. Examples of mimic TCRs include but are not limited to, TCRm Abs, TCRm BITEs, TCRm CARs, and ImmTACs. In some embodiments, the TCR is 1G4, DMF5, or A6 TCR or an affinity enhanced version thereof.

In some embodiments, engineered immune cells of the present disclosure comprises an engineered cytotoxic T cell that lacks detectable expression or activity of a wild-type CD8α polypeptide. In some such embodiments, the wild-type CD8α polypeptide comprises the amino acid sequence of SEQ ID NO: 36 or SEQ ID NO: 47. In some embodiments, the engineered cytotoxic T cell comprises a deletion, an inversion, a missense mutation, a nonsense mutation, or a frameshift mutation in a nucleic acid sequence encoding the wild-type CD8α polypeptide (i.e., wherein the nucleic acid sequence encoding the wild-type CD8α polypeptide is SEQ ID NO: 33) or comprises one or more disruptions in endogenous genes encoding the wild-type CD8α polypeptide (e.g., CRISPR knockouts). By way of example only, one or more endogenous genes may be knocked out using an endonuclease selected from the group consisting of a CRISPR system (e.g., a Cas endonuclease), TALEN, Zinc Finger, transposon-based, ZEN, meganuclease, Mega-TAL, and any combination thereof.

Gene suppression can be performed in a number of ways. For example, gene expression can be suppressed by knock out, altering a promoter of a gene, and/or by inhibiting transcriptional or translational activity. This can be done at an organism level or at a tissue, organ, and/or cellular level. Gene suppression methods may comprise overexpressing a dominant negative protein. This method can result in overall decreased function of a functional wild-type gene. Additionally, expressing a dominant negative gene can result in a phenotype that is similar to that of a knockout and/or knockdown. Sometimes a stop codon can be inserted or created (e.g., by nucleotide replacement), in one or more genes, which can result in a nonfunctional transcript or protein (sometimes referred to as knockout). For example, if a stop codon is created within the middle of one or more genes, the resulting transcription and/or protein can be truncated, and can be nonfunctional. However, in some cases, truncation can lead to an active (a partially or overly active) protein. If a protein is overly active, this can result in a dominant negative protein.

In some embodiments, the engineered cytotoxic T cell comprises an inhibitory nucleic acid that specifically targets and inhibits the expression of a nucleic acid sequence encoding the wild-type CD8α polypeptide (i.e., wherein the nucleic acid sequence encoding the wild-type CD8α polypeptide is SEQ ID NO: 33). In some such embodiments, the inhibitory nucleic acid is an antisense oligonucleotide, a siRNA, a sgRNA or a shRNA.

In some embodiments, engineered immune cells of the present disclosure comprise an engineered cytotoxic T cell that comprises a non-endogenous expression vector that includes a nucleic acid sequence encoding a mutant CD8α polypeptide (i.e., comprising the amino acid sequence of SEQ ID NO: 37, SEQ ID NO: 38, SEQ ID NO: 48, or SEQ ID NO: 49). In some embodiments, the nucleic acid sequence encoding a mutant CD8α polypeptide includes, for example, SEQ ID NO: 34 or SEQ ID NO: 35. In some embodiments, the mutant CD8α polypeptide exhibits reduced binding to MHC relative to the wild-type CD8α polypeptide. In some embodiments, the mutant CD8α polypeptide exhibits increased binding to MHC relative to the wild-type CD8α polypeptide.

In some embodiments, engineered immune cells of the present disclosure comprise an engineered CD4+ helper T cell that comprises a non-endogenous expression vector that includes a nucleic acid sequence encoding a CD8α polypeptide (i.e., comprising the amino acid sequence of SEQ ID NO: 36, SEQ ID NO: 37, SEQ ID NO: 38, SEQ ID NO: 47, SEQ ID NO: 48, or SEQ ID NO: 49). In some embodiments, the nucleic acid sequence encoding a CD8α polypeptide, includes, for example, SEQ ID NO: 33, SEQ ID NO: 34, or SEQ ID NO: 35.

In some embodiments, engineered immune cells, as described herein, express a heterologous amino acid sequence that is at least 80%, 85%, 90%, 95%, 96%, 97%, 98%, or 99% identical to SEQ ID NO: 36, SEQ ID NO: 37, SEQ ID NO: 38, SEQ ID NO: 47, SEQ ID NO: 48, or SEQ ID NO: 49 or a biological equivalent thereof. In further embodiments, a biological equivalent of SEQ ID NO: 36, SEQ ID NO: 37, SEQ ID NO: 38, SEQ ID NO: 47, SEQ ID NO: 48, or SEQ ID NO: 49 comprises one or more conservative amino acid substitutions relative to SEQ ID NO: 36, SEQ ID NO: 37, SEQ ID NO: 38, SEQ ID NO: 47, SEQ ID NO: 48, or SEQ ID NO: 49 respectively. Additionally or alternatively, in some embodiments, the biological equivalent comprises CD8α activity (e.g., binding to MHC) substantially similar or significantly more compared to the polypeptide of SEQ ID NO: 36 or SEQ ID NO: 47.

In some embodiments, engineered immune cells, as described herein, express a heterologous nucleic acid sequence that is at least 80%, 85%, 90%, 95%, 96%, 97%, 98%, or 99% identical to SEQ ID NO: 33, SEQ ID NO: 34, or SEQ ID NO: 35. Additionally or alternatively, in some embodiments, the expression levels and/or activity of CD8α polypeptide in an engineered immune cells is at least 2, at least 3, at least 4, at least 5, at least 6, at least 7, at least 8, at least 9, at least 10, at least 20, at least 30, at least 40, at least 50, at least 60, at least 70, at least 80, at least 90, at least 100, at least 200, at least 300, at least 400, at least 500, at least 600, at least 700, at least 800, at least 900, or at least 1000 times higher compared to that observed in a native immune cell, wherein the engineered immune cell is of the same lineage as the native immune cell.

In certain embodiments, the presently disclosed engineered immune cells (e.g., T cells) expresses from about 1 to about 5, from about 1 to about 4, from about 2 to about 5, from about 2 to about 4, from about 3 to about 5, from about 3 to about 4, from about 4 to about 5, from about 1 to about 2, from about 2 to about 3, from about 3 to about 4, or from about 4 to about 5 vector copy numbers per cell of a CD8α heterologous nucleic acid and/or TCR heterologous nucleic acid.

In some embodiments, engineered immune cells of the presently disclosed technologies express non-endogenous levels of CD8α (including, e.g., a mutant CD8α) and/or levels of a TCR for the treatment of cancer, e.g., for treatment of tumor. In some such embodiments, engineered immune cells are administered to a subject (e.g., a human subject) in need thereof for the treatment of cancer.

In some embodiments, the present disclosure provides a composition comprising an effective amount of engineered cytotoxic T cell or engineered CD4+ helper T cells and a pharmaceutically acceptable carrier.

In some embodiments, technologies of the present disclosure provide a method for mitigating off-target reactivity and/or toxicity in a subject receiving adoptive T cell therapy comprising administering to a subject in need thereof, an effective amount of engineered cytotoxic T cells, engineered CD4+ helper T cell, or engineered cytotoxic T cells or engineered CD4+ helper T cell and a pharmaceutically acceptable carrier.

In some embodiments, engineered immune cells of the present disclosure may further include at least one recombinant or exogenous co-stimulatory ligand. For example, the presently disclosed engineered immune cells can be further transduced with at least one co-stimulatory ligand, such that the engineered immune cells co-expresses or is induced to co-express mutant CD8α and/or a TCR (e.g., 1G4 TCR) and the at least one co-stimulatory ligand. Co-stimulatory ligands include, but are not limited to, members of the tumor necrosis factor (TNF) superfamily, and immunoglobulin (Ig) superfamily ligands. TNF is a cytokine involved in systemic inflammation and stimulates the acute phase reaction. Its primary role is in the regulation of immune cells. Members of TNF superfamily share a number of common features. The majority of TNF superfamily members are synthesized as type II transmembrane proteins (extracellular C-terminus) containing a short cytoplasmic segment and a relatively long extracellular region. TNF superfamily members include, without limitation, nerve growth factor (NGF), CD40L (CD40L)/CD 154, CD137L/4-1BBL, TNF-α, CD134L/OX40L/CD252, CD27L/CD70, Fas ligand (FasL), CD30L/CD153, tumor necrosis factor beta (TNFP)/lymphotoxin-alpha (LTa), lymphotoxin-beta O-TB), CD257/B cell-activating factor (B AFF)/Bly s/THANK/Tall-1, glucocorticoid-induced TNF Receptor ligand (GITRL), and T F-related apoptosis-inducing ligand (TRAIL), LIGHT (TNFSF14). The immunoglobulin (Ig) superfamily is a large group of cell surface and soluble proteins that are involved in the recognition, binding, or adhesion processes of cells. These proteins share structural features with immunoglobulins—they possess an immunoglobulin domain (fold). Immunoglobulin superfamily ligands include, but are not limited to, CD80 and CD86, both ligands for CD28, PD-L1/(B7-H1) that ligands for PD-1. In certain embodiments, the at least one co-stimulatory ligand is selected from the group consisting of 4-1BBL, CD80, CD86, CD70, OX40L, CD48, TNFRSF14, PD-L1, and combinations thereof. In certain embodiments, the engineered immune cell comprises one recombinant co-stimulatory ligand (e.g., 4-1BBL). In certain embodiments, the engineered immune cell comprises two recombinant co-stimulatory ligands (e.g., 4-1BBL and CD80).

Furthermore, the presently disclosed engineered immune cells can, in some embodiments, further comprise at least one exogenous cytokine. For example, a presently disclosed engineered immune cell can be further transduced with at least one cytokine, such that the engineered immune cells secrete the at least one cytokine as well as express mutant CD8α. In certain embodiments, the at least one cytokine is selected from the group consisting of IL-2, IL-3, IL-6, IL-7, IL-11, IL-12, IL-15, IL-17, and IL-21. In certain embodiments, the cytokine is IL-12.

The engineered immune cells (e.g., T cells) can be generated from peripheral donor lymphocytes, e.g., those disclosed in Sadelain, M., et al., Nat Rev Cancer 3:35-45 (2003), in Morgan, R. A. et al. (2006) Science 314:126-129; in Dupont et al. (2005) Cancer Res 65:5417-5427; Papanicolaou et al. (2003) Blood 102:2498-2505. The engineered immune cells (e.g., T cells) can be autologous, non-autologous (e.g., allogeneic), or derived in vitro from engineered progenitor or stem cells.

The unpurified source of immune cells can be any known in the art, such as the bone marrow, fetal, neonate or adult or other hematopoietic cell source, e.g., fetal liver, peripheral blood or umbilical cord blood. Various techniques can be employed to separate the cells. For instance, negative selection methods can remove non-immune cell initially. Monoclonal antibodies are particularly useful for identifying markers associated with particular cell lineages and/or stages of differentiation for both positive and negative selections.

A large proportion of terminally differentiated cells can be initially removed by a relatively crude separation. For example, magnetic bead separations can be used initially to remove large numbers of irrelevant cells. In some embodiments, at least about 80%, usually at least 70% of the total hematopoietic cells will be removed prior to cell isolation.

Procedures for separation include, but are not limited to, density gradient centrifugation; resetting; coupling to particles that modify cell density; magnetic separation with antibody-coated magnetic beads; affinity chromatography; cytotoxic agents joined to or used in conjunction with a mAb, including, but not limited to, complement and cytotoxins; and panning with antibody attached to a solid matrix, e.g., plate, chip, elutriation or any other convenient technique.

Techniques for separation and analysis include, but are not limited to, flow cytometry, which can have varying degrees of sophistication, e.g., a plurality of color channels, low angle and obtuse light scattering detecting channels, impedance channels.

The cells can be selected against dead cells, by employing dyes associated with dead cells such as propidium iodide (PI). In some embodiments, the cells are collected in a medium comprising 2% fetal calf serum (FCS) or 0.2% bovine serum albumin (BSA) or any other suitable, preferably sterile, isotonic medium.

In some embodiments, the engineered immune cells comprise one or more additional modifications. For example, in some embodiments, the engineered immune cells comprise and express (is transduced to express) a chimeric co-stimulatory receptor (CCR). CCR is described in Krause et al. (1998) J. Exp. Med. 188 (4): 619-626, and US20020018783, the contents of which are incorporated by reference in their entireties. CCRs mimic co-stimulatory signals, but unlike, engineered receptors, do not provide a T-cell activation signal, e.g., CCRs lack a CD3ζ polypeptide. CCRs provide co-stimulation, e.g., a CD28-like signal, in the absence of the natural co-stimulatory ligand on the antigen-presenting cell. A combinatorial antigen recognition, i.e., use of a CCR in combination with an engineered receptor, can augment T-cell reactivity against the dual-antigen expressing T cells, thereby improving selective tumor targeting.

In some embodiments, the engineered immune cells are further modified to suppress expression of one or more genes. In some embodiments, the engineered immune cells are further modified via genome editing. Various methods and compositions for targeted cleavage of genomic DNA have been described. Such targeted cleavage events can be used, for example, to induce targeted mutagenesis, induce targeted deletions of cellular DNA sequences, and facilitate targeted recombination at a predetermined chromosomal locus. See, for example, U.S. Pat. Nos. 7,888,121; 7,972,854; 7,914,796; 7,951,925; 8,110,379; 8,409,861; 8,586,526; U.S. Patent Publications 20030232410; 20050208489; 20050026157; 20050064474; 20060063231; 201000218264; 20120017290; 20110265198; 20130137104; 20130122591; 20130177983 and 20130177960, the disclosures of which are incorporated by reference in their entireties. These methods often involve the use of engineered cleavage systems to induce a double strand break (DSB) or a nick in a target DNA sequence such that repair of the break by an error born process such as non-homologous end joining (NHEJ) or repair using a repair template (homology directed repair or HDR) can result in the knock out of a gene or the insertion of a sequence of interest (targeted integration). Cleavage can occur through the use of specific nucleases such as engineered zinc finger nucleases (ZFN), transcription-activator like effector nucleases (TALENs), or using the CRISPR/Cas system with an engineered crRNA/tracr RNA (‘single guide RNA’) to guide specific cleavage. In some embodiments, the engineered immune cells are modified to disrupt or reduce expression of an endogenous T-cell receptor gene (see, e.g., WO 2014153470, which is incorporated by reference in its entirety). In some embodiments, the engineered immune cells are modified to result in disruption or inhibition of PD1, PDL-1 or CTLA-4 (see, e.g. U.S. Patent Publication 20140120622), or other immunosuppressive factors known in the art (Wu et al. (2015) Oncoimmunology 4(7): e1016700, Mahoney et al. (2015) Nature Reviews Drug Discovery 14, 561-584).

### Administration

Engineered immune cells expressing TCRs and/or CD8α polypeptides of the presently disclosed subject matter can be provided systemically or directly to a subject for treating a disease or condition (e.g., cancer). In some embodiments, a subject suffers from or is diagnosed with cancer. In some embodiments, the cancer or tumor is selected from the group consisting of adrenal cancers, bladder cancers, blood cancers, bone cancers, brain cancers, breast cancers, carcinoma, cervical cancers, colon cancers, colorectal cancers, corpus uterine cancers, ear, nose and throat (ENT) cancers, endometrial cancers, esophageal cancers, gastrointestinal cancers, head and neck cancers, Hodgkin's disease, intestinal cancers, kidney cancers, larynx cancers, acute and chronic leukemias, liver cancers, lymph node cancers, lymphomas, lung cancers, melanomas, mesothelioma, myelomas, nasopharynx cancers, neuroblastomas, non-Hodgkin's lymphoma, oral cancers, ovarian cancers, pancreatic cancers, penile cancers, pharynx cancers, prostate cancers, rectal cancers, sarcoma, seminomas, skin cancers, stomach cancers, teratomas, testicular cancers, thyroid cancers, uterine cancers, vaginal cancers, vascular tumors, and metastases thereof.

In some embodiments, technologies of the present disclosure provide a method for treating cancer or inhibiting tumor growth in a subject in need thereof comprising administering to the subject an effective amount of an engineered cytotoxic T cell, an engineered CD4+ helper T cell, or a composition comprising an effective amount of an engineered cytotoxic T cell or an engineered CD4+ helper T cell and a pharmaceutically acceptable carrier as disclosed herein.

In certain embodiments, engineered immune cells or compositions comprising engineered immune cells are directly injected into an organ of interest (e.g., a tissue affected by cancer). Additionally or alternatively, engineered immune cells or compositions thereof are provided indirectly to the organ of interest, for example, by administration into the circulatory system or into the tissue of interest. In certain embodiments, engineered immune cells or compositions thereof are injected intratumorally. Expansion and differentiation agents can be provided prior to, during or after administration of cells and compositions to increase production of T cells in vitro or in vivo.

Engineered immune cells of the presently disclosed subject matter or compositions thereof can be administered in any physiologically acceptable vehicle, systemically or regionally, normally intravascularly, intraperitoneally, intrathecally, or intrapleurally, although they may also be introduced into bone or other convenient site where the cells may find an appropriate site for regeneration and differentiation (e.g., thymus). In certain embodiments, at least 1×10⁵ cells can be administered, eventually reaching 1×10¹⁰ or more. In certain embodiments, at least 1×10⁶ cells can be administered. A cell population comprising engineered immune cells can comprise a purified population of cells. Those skilled in the art can readily determine the percentage of engineered immune cells in a cell population using various well-known methods, such as fluorescence activated cell sorting (FACS). The ranges of purity in cell populations comprising engineered immune cells can be from about 50% to about 55%, from about 55% to about 60%, about 60% to about 65%, from about 65% to about 70%, from about 70% to about 75%, from about 75% to about 80%, from about 80% to about 85%; from about 85% to about 90%, from about 90% to about 95%, or from about 95 to about 100%. Dosages can be readily adjusted by those skilled in the art (e.g., a decrease in purity may require an increase in dosage). The engineered immune cells or compositions thereof can be introduced by injection, catheter, or the like. If desired, factors can also be included, including, but not limited to, interleukins, e.g., IL-2, IL-3, IL 6, IL-11, IL-7, IL-12, IL-15, IL-21, as well as the other interleukins, the colony stimulating factors, such as G-, M- and GM-CSF, interferons, e.g., γ-interferon.

In certain embodiments, compositions of the presently disclosed subject matter comprise pharmaceutical compositions comprising engineered immune cells expressing certain TCRs and/or CD8α polypeptides as described herein with a pharmaceutically acceptable carrier. Administration can be autologous or non-autologous. For example, engineered immune cells expressing TCRs and/or CD8α polypeptides of the present disclosure and compositions comprising the same can be obtained from one subject (e.g., a donor subject), and administered to the same subject or a different, compatible subject (e.g., a recipient subject). Peripheral blood derived T cells of the presently disclosed subject matter or their progeny (e.g., in vivo, ex vivo or in vitro derived) can be administered via localized injection, including catheter administration, systemic injection, localized injection, intravenous injection, or parenteral administration. When administering a pharmaceutical composition of the presently disclosed subject matter (e.g., a pharmaceutical composition comprising engineered immune cells expressing TCRs and/or CD8 molecules of the present disclosure, it can be formulated in a unit dosage injectable form (solution, suspension, emulsion).

### Formulations

Engineered immune cells of the present disclosure and compositions comprising the same can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Sterile injectable solutions can be prepared by incorporating the compositions of the presently disclosed subject matter, e.g., a composition comprising engineered immune cells, in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin. According to the presently disclosed subject matter, however, any vehicle, diluent, or additive used would have to be compatible with the engineered immune cells of the presently disclosed subject matter.

The compositions can be isotonic, i.e., they can have the same osmotic pressure as blood and lacrimal fluid. The desired isotonicity of the compositions of the presently disclosed subject matter may be accomplished using sodium chloride, or other pharmaceutically acceptable agents such as dextrose, boric acid, sodium tartrate, propylene glycol or other inorganic or organic solutes. Sodium chloride is suitable particularly for buffers containing sodium ions.

Viscosity of the compositions, if desired, can be maintained at the selected level using a pharmaceutically acceptable thickening agent. Methylcellulose can be used because it is readily and economically available and is easy to work with. Other suitable thickening agents include, for example, xanthan gum, carboxymethyl cellulose, hydroxypropyl cellulose, carbomer, and the like. The concentration of the thickener can depend upon the agent selected. The important point is to use an amount that will achieve the selected viscosity. Obviously, the choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form).

Those skilled in the art will recognize that the components of the compositions should be selected to be chemically inert and will not affect the viability or efficacy of the engineered immune cells as described in the presently disclosed subject matter. This will present no problem to those skilled in chemical and pharmaceutical principles, or problems can be readily avoided by reference to standard texts or by simple experiments (not involving undue experimentation), from this disclosure and the documents cited herein.

One consideration concerning the therapeutic use of the engineered immune cells of the presently disclosed subject matter is the quantity of cells necessary to achieve an optimal effect. The quantity of cells to be administered will vary for the subject being treated. In certain embodiments, from about 10² to about 10¹², from about 10³ to about 10¹¹, from about 10⁴ to about 10¹⁰, from about 10⁵ to about 10⁹, or from about 10⁶ to about 10⁸ engineered immune cells of the presently disclosed subject matter are administered to a subject. More effective cells may be administered in even smaller numbers. In some embodiments, at least about 1×10⁸, about 2×10⁸, about 3×10⁸, about 4×10⁸, about 5×10⁸, about 1×10⁹, about 5×10⁹, about 1×10¹⁰, about 5×10¹⁰, about 1×10¹¹, about 5×10¹¹, about 1×10¹² or more engineered immune cells of the presently disclosed subject matter are administered to a human subject. The precise determination of what would be considered an effective dose may be based on factors individual to each subject, including their size, age, sex, weight, and condition of the particular subject. Dosages can be readily ascertained by those skilled in the art from this disclosure and the knowledge in the art. Generally, engineered immune cells are administered at doses that are nontoxic or tolerable to the patient.

The skilled artisan can readily determine the amount of cells and optional additives, vehicles, and/or carrier in compositions to be administered in methods of the presently disclosed subject matter. Typically, any additives (in addition to the active cell(s) and/or agent(s)) are present in an amount of from about 0.001% to about 50% by weight) solution in phosphate buffered saline, and the active ingredient is present in the order of micrograms to milligrams, such as from about 0.0001 wt % to about 5 wt %, from about 0.0001 wt % to about 1 wt %, from about 0.0001 wt % to about 0.05 wt %, from about 0.001 wt % to about 20 wt %, from about 0.01 wt % to about 10 wt %, or from about 0.05 wt % to about 5 wt %. For any composition to be administered to an animal or human, and for any particular method of administration, toxicity should be determined, such as by determining the lethal dose (LD) and LD50 in a suitable animal model e.g., rodent such as mouse; and, the dosage of the composition(s), concentration of components therein and timing of administering the composition(s), which elicit a suitable response. Such determinations do not require undue experimentation from the knowledge of the skilled artisan, this disclosure and the documents cited herein. And, the time for sequential administrations can be ascertained without undue experimentation.

### Combination Therapy

Also provided are methods for treating cancer in a subject in need thereof comprising administering to the subject an effective amount of any of the engineered immune cells provided herein. In some embodiments of the methods disclosed herein, the engineered immune cell(s) are administered systemically, intranasally, intrapleurally, intravenously, intraperitoneally, subcutaneously, or intramuscularly. In some embodiments, the subject in need thereof is human.

Methods for treating cancer may further comprise sequentially, separately, or simultaneously administering to the subject at least one additional therapy selected from among chemotherapy, radiation, and/or immune modulators.

In some embodiments, immune modulators comprise immune checkpoint modulators. In some embodiments, immune checkpoint modulators comprise, for example, anti-CTLA4 antibodies (e.g., ipilimumab), anti-PD-1 and/or anti-PD-L1 antibodies (e.g., atezolizumab, avelumab, cemiplimab, dostarlimab, durvalumab, nivolumab, pembrolizumab), and/or anti-LAG-3 antibodies (e.g., Relatlimab).

In some embodiments, immune modulators comprise cytokine-based therapies. In some such embodiments, cytokine-based therapies comprise, for example, aldesleukin, Granulocyte-macrophage colony-stimulating factor (GM-CSF), interferon-α2a, interferon-α2b, and/or peginterferon alfa-2b.

In some embodiments, immune modulators comprise adjuvants. In some such embodiments, adjuvants comprise, for example, imiquimod and/or poly ICLC.

Additionally or alternatively, in some embodiments, methods for treating cancer in a subject in need thereof comprises administering engineered immune cells as described herein to a subject that has received or is receiving radiation therapy, chemotherapy, or a combination of radiation therapy and chemotherapy.

Methods for treating cancer may further comprise sequentially, separately, or simultaneously administering to the subject at least one additional therapy selected from among bevacizumab, irinotecan hydrochloride, capecitabine, cetuximab, ramucirumab, fluorouracil, ipilimumab, pembrolizumab, leucovorin calcium, trifluridine and tipiracil Hydrochloride, nivolumab, oxaliplatin, panitumumab, regorafenib, and ziv-aflibercept.

In any case, the multiple therapeutic agents may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may vary from more than zero weeks to less than four weeks. In addition, the combination methods, compositions and formulations are not to be limited to the use of only two agents.

### Kits

In one aspect, the kits of the present technology comprise a therapeutic composition including any of the engineered immune cells disclosed herein in unit dosage form, and/or vectors comprising any of the nucleic acids disclosed herein. In some embodiments, the kit comprises a sterile container which contains therapeutic compositions including the engineered immune cells disclosed herein; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

In some embodiments of the kits, the engineered immune cells of the present technology can be provided together with instructions for administering the engineered immune cell to a subject. In some embodiments, the subject is diagnosed with or suffers from cancer. In certain embodiments of the kits, the vectors comprising any of the nucleic acids disclosed herein can be provided together with instructions for using immune cells transduced with said vectors to treat or mitigate any disease or condition described herein. In certain embodiments of the kits, the vectors comprising any of the nucleic acids disclosed herein can be provided together with instructions for transducing CD4+ helper T cells or cytotoxic T cells with expression vector.

The instructions will generally include information about the use of the composition for the treatment of any disease or condition described herein. In other embodiments, the instructions include at least one of the following: description of the therapeutic agent; dosage schedule and administration for treatment of any disease or condition described herein or symptoms thereof; precautions; warnings; indications; counter-indications; overdose information; adverse reactions; animal pharmacology; clinical studies; and/or references. The instructions may be printed directly on the container (when present), or as a label applied to the container, or as a separate sheet, pamphlet, card, or folder supplied in or with the container.

The at least one engineered immune cell of the present technology may be provided in the form of a prefilled syringe or autoinjection pen containing a sterile, liquid formulation or lyophilized preparation (e.g., Kivitz et al., Clin. Ther. 28:1619-29 (2006)).

A device capable of delivering the kit components through an administrative route may be included. Examples of such devices include syringes (for parenteral administration) or inhalation devices.

The kit components may be packaged together or separated into two or more containers. In some embodiments, the containers may be vials that contain sterile, lyophilized formulations of engineered immune cell compositions of the present technology that are suitable for reconstitution. A kit may also contain one or more buffers suitable for reconstitution and/or dilution of other reagents. Other containers that may be used include, but are not limited to, a pouch, tray, box, tube, or the like. Kit components may be packaged and maintained sterilely within the containers.

### EXAMPLES

### Example 1: General Experimental Methods

Cell lines and cell culture: The human lymphoblast cell line T2 was previously obtained from ATCC. This cell line was cultured in IMDM with penicillin/streptomycin, NEAA, 10% FBS and 1% Glutamine. T2 cells are a suspension cell line and were cultured in vented flasks. The HEK-Ampho cell line was cultured in either IMDM or DMEM, both with penicillin/streptomycin, NEAA, 10% FBS and 1% glutamine. HEK-Ampho cells are an adherent cell line and were cultured in tissue culture treated plates.

PresentER construct generation, single minigene: Minigene constructs were ordered from IDT with the following sequence, GGCCGTATTGGCCCCGCCACCTGTGAGCGGGPEPTIDETAAGGCCAAACAGGCC (“GGCCGTATTGGCCCCGCCACCTGTGAGCGGG” disclosed as SEQ ID NO: 13 and “TAAGGCCAAACAGGCC” disclosed as SEQ ID NO: 106). Minigenes were amplified with 2×Q5 master mix from New England Bio Labs (catalog #M0494) with the following primers: CGACTCACTATAGGGCCGTATTGGCC (SEQ ID NO: 14) and AGTGATTTCCGGCCTGTTTGGCC (SEQ ID NO: 15). PCR sequences were digested with SfiI enzyme at 50° C. Minigenes were then purified with the Qiagen MinElute kit (catalog #28004). PresentER vector was digested with SfiI enzyme at 50° C. and then CIP enzyme at 37° C. and purified by gel extraction with the Qiagen Gel Purification kit (catalog #28706). Minigenes were ligated to digested backbone at 5 moles: 1 mole ratio of insert to backbone with T4 ligase at 16° C. overnight.

PresentER construct generation, libraries: Pooled oligos were ordered with the sequence as described for single minigenes with the addition of DNA extensions where library specific forward and reverse primers can bind. Each library was amplified over twelve 50 μL PCRs with library specific outer primers with 2×Q5 master mix from New England Bio Labs (catalog #M0494). PCR products were purified by ethanol precipitation. 2 ng of PCR product in nuclease free water was amplified with the following primers, GACCTCCCTCACGCTGTTTCTAGCACTCTTGGCCGTATTGGCCCCG (SEQ ID NO: 16) and CTTTCCCTACACGACGCTCTTCCGATCTTTGGCCTGTTTGGCCTTA (SEQ ID NO: 17), over two 50 μL PCRs with 2×Q5 master mix. PCR product was purified with Qiagen MinElute kit (catalog #28004). Five 20 μL ligation reactions were set up with 20-40 ng of PCR product and 100 ng digested PresentER backbone (generation described above in single minigene generation) with 2×Gibson Assembly master mix from New England BioLabs (catalog #E2611). Ligation reactions were pooled and DNA extracted via phenol extraction and resuspended in 3 μL Elution Buffer (EB). DNA was mixed with 50 μL MegaX DH10B™ TIR Electrocompetant Cells from Thermo Fischer Scientific (catalog #C640003) and electroporated in 1 mm cuvettes at 2 KV for 6 msec. Bacteria were transferred to SOC and incubated in shaker at 37° C. for one hour before being plated over four 15 cm ampicillin plates and incubated overnight. Bacteria were scraped off plates and moved to 350 mL terrific broth with ampicillin and allowed to shake at 37° C. before the DNA was extracted with the PureLink™ HiPure Plasmid Filter Maxiprep Kit from Thermo Fischer Scientific (catalog #K210017). Minigene sequences were amplified as described below. PCR amplicons were analyzed by Illumina next generation sequencing to validate library generation.

PresentER Library Design: A02 binding affinity of 9mer peptides from the human proteome (UniProtKB/Swiss-Prot. Downloaded on May 7, 2017) were predicted using NetMHCpan-4.0. Peptides with a binding affinity below 500 nM were then scored for similarity to NY-ESO-1 peptide using the PMBEC amino acid similarity scoring matrix, excluding positions 2 and 9. Peptides were ranked by similarity to determine inclusion in the library, with low molecular weight peptides prioritized to choose 5,000 peptides for library inclusion. An additional ˜1,000 peptides were included. 9mer and 10mer peptides from the human proteome with a MW motif flanked by three peptides on either side (for 9mers: XXXMWXXXX or XXXXMWXXX; for 10mers: XXXMWXXXXX, XXXXMWXXXX, or XXXXXMWXXX) were prioritized by low molecular weight for inclusion in the library. Only unique peptides were included in the final library design.

PresentER Library coculture screens: Library cells were assessed for percent library positive (GFP positive) by flow cytometry. Enough cells for 1000× library coverage or 10e6 target cells, whichever was higher, was used in each library screen. Target library cells were mixed 1:1 with effector T cells, either transduced or untransduced with the TCR of interest, in 20 mL of RPMI final volume. The cell mixture was distributed in a 96-well round bottom plate (˜200 μL/well) (day 0). On days 1, 2, and 3 the cells were removed, pooled and replated. When the media yellowed, usually on day 1 or 2, the cells were removed, pooled and 60 mL of additional RPMI was added. The cells were replated over four 96-well plates (˜200 μL/well). On day 4 the cells were harvested and pooled. Cells were spun down and immediately frozen or DNA extracted. DNA was extracted with Qiagen Gentra Puregene Tissue Kit (catalog #158689).

Library POR for next generation sequencing: Extracted DNA from screens or library generation was amplified by nested PCR. DNA was first amplified with AATGATACGGCGACCACCGAGATCT (SEQ ID NO: 18) and GTGACTGGAGTTCAGACGTGTGCTCTTCCGATC (SEQ ID NO: 19) primers using 2×Q5 master mix from NEB in 50 μL reactions. All DNA extracted from screens was amplified using 5-10 μg extracted DNA per reaction. 2-3 PCR reactions using Ing of DNA per reaction was done for library sequence validation. PCR products were purified via MinElute. Samples were pooled and brought to 2 ng/μL. 2 ng of PCR product were then amplified per reaction in two 50 μL PCRs using GCCACCTGTGAGCGGG (SEQ ID NO: 20) and TCTTTGGCCTGTTTGGCCTTA (SEQ ID NO: 21) primers and 2×Q5 master mix. PCRs were purified by MinElute and sent for library preparation and Illumina next generation sequencing by the Memorial Sloan Kettering Cancer Center IGO Genomics Core Facility.

Single Antigen Coculture Screens: T cells were washed twice to remove IL2 from medium. Equal numbers of T2 cells and T cells were mixed together at 1e6 cells/mL and plated in triplicate across three wells of a 96-well round bottom plate. Each well contained 200 μL total volume. Cytotoxicity was analyzed by flow cytometry, detecting GFP cells, which were antigen positive at 24-72 hours.

Elispot: MultiScreen-IP filter plates (Millipore Sigma #S2EM004M99) were pre-wet with 15 μL/well 70% ethanol. Anti-IFN clone 1-DIK (Mabtech #3420-3-1000) was diluted to 10 μg/mL with PBS, 100 μL was added to each well of plate and incubated at 4° C. overnight. The plates were washed with PBS and then blocked with 150 μL complete RPMI for 2 hours at 37° C. T cells were washed two times to remove cytokines and resuspended at 0.5e6 cells/mL in complete RPMI. 100 μL of T cells were added to each well. Target cells were pulsed with peptide at 20 μg/mL for 2 hours at 37° C. Target cells were washed and resuspended at 0.1e6 cells/mL in complete RPMI. 100 μL target cells were added on top of T cells. 100 μL of media was added to control wells. Phytohemagglutinin (PHA) was added to wells for a final concentration of 10 μg/mL. Plate was incubated at 37° C. for >20 hours. Plate was then washed six times with PBS/0.05% Tween20 and three times with PBS. Anti-IFN-gamma-biotin clone 7-B6-1 (Mabtech #3420-6-250) was diluted in PBS/0.5% FBS to 2 μg/mL, 100 μL was added to each well and incubated for 2 hours at 37° C. Plate was washed three times with PBS/0.05% Tween20 and three times with PBS. 100 μL of Vectastain Elite ABC HRP Detection Kit (Vector Laboratories #PK-6100) was prepared in PBS/0.1% Tween20 and was added to each well and incubated for 1 hour at room temperature. The plate was then washed three times with PBS/0.05% Tween20 and three times with PBS. The following substrates were prepared, substrates 1:23.4 mL deionized (DI) water, 2.3 mL 0.1N acetic acid, 5.5 mL 0.1N sodium acetate; substrate 2:2.5 mL dimethylformamide and one 3-Amino-9-ethylcarbazole tablet (Sigma #A6926). 23.75 mL of solution 1 of the HRP kit was mixed with 1.25 mL of solution 2 of the HRP kit, 12.5 μL 30% H₂O₂ was added to the mixture. 100 μL of substrate mixture was added to each well and incubated for 4 minutes at room temperature. Reaction was stopped with water. Plates were allowed to dry completely before spots were counted.

ELISpot Assays with Blocking Antibodies: ELISpot was performed as described previously except T cells were preincubated with anti-CD8 or anti-CD4 antibodies for at least 1 hour at 5 μg/mL prior to plating. Anti-CD8 antibodies 3B5 (Thermo scientific #MHCD0800) and OKT8 (ThermoFisher Scientific #14-0086-80) and anti-CD4 antibody OKT4 (ThermoFisher Scientific #14-0048-82) were used.

T cell processing: Whole blood processing: Whole blood was donated by healthy donors. Lymphocyte separation medium (Fischer Scientific #MT25072CI) was added to 50 mL Accuspin tubes (Sigma-Aldrich #A2055) and spun to move medium to the bottom chamber of the tube. Whole blood was added to upper tube chamber. Tubes were spun at 2000 rpm for 20 minutes with no break. Buffy coat was removed. Cells were washed with PBS one time. Cells were resuspended in 2 mL ACK lysis buffer (Thermo Fischer Scientific #A10492-01) and incubated for 5 minutes at room temperature. Samples were washed with PBS one time and resuspended in complete RPMI. T cells were stimulated with 50 ng/ml OKT3 and 200 IU/mL IL2. Fresh IL2 at 200 IU/mL was added every two days.

CD4 and CD8 cell Isolation Depletion: To separate CD4 and CD8 cells through negative selection, magnetic microbeads were used. CD8 beads (Miltenyi Biotec #130-045-201) were used to isolate CD4 cells (and remove CD8-expressing cells after CD8 knockout via CRISPR/Cas9) and CD4 beads (Miltenyi Biotec #130-045-101) were used to isolate CD8 cells. Microbeads were used as described in the manufacturer's protocol. Briefly, T cells were counted and resuspended in PBS/0.5% FBS/2 mM EDTA at 10e7 cells/mL. Cells were incubated with 20 μL microbeads per 10e7 cells for 15 minutes at 4° C. LD columns were put in a MACS separator, cells were added to the column and allowed to flow through by gravity. Columns were washed two times with 1 mL of PBS/0.5% FBS/2 mM EDTA. Cells were spun and resuspended in RPMI. T cells were separated prior to stimulation with OKT3 and IL2.

MACS separation: MACS buffer: PBS/0.5% FBS/2 mM EDTA To separate CD4 negative T cells, CD4 beads (Miltenyi Biotec #130-045-101) were used. Beads were used as described in the manufacturer's protocol. To separate CD8β negative T cell, 2 μL CD8β antibody Biotin REAfinity (Miltenyi Biotec #130-110-508) was incubated with 10e6 washed T cells resuspended in 98 μL MACS buffer for 10 minutes at 4° C. After washing with MACS buffer, the cells were resuspended in 90 μL MACS buffer and incubated with 10 μL Streptavidin MicroBeads (Miltenyi Biotec #130-048-101) for 15 minutes at 4° C. LD columns were put in a MACS separator, cells were added to the column and allowed to flow through by gravity. Columns are washed two times with 1 mL of MACS buffer. Cells were spun and resuspended in RPMI.

T cell transduction: Producer cell line (Galv-9) were transduced using 1.5 mL of 1G4 retrovirus. T cells were transduced using 6-well non-tissue culture treated plates coated in retronectin for 30 minutes at 37° C. or overnight at 4° C. Plates were washed one time with PBS. 1 mL filtered supernatant of Galv-9-1G4 cells was added to each well. 2e6 T cells (2-3 days post stimulation) were added to each well. Plates are centrifuged at 2000 g for 90 minutes at 32° C.

Primary T cell Transduction: 6 well non-tissue culture treated plates were coated in retronectin for 30 minutes to 2 hours at 37° C. or overnight at 4° C. Plates were washed one time with PBS. 1 mL virus was added to each well. 1-2e6 T cells (2-3 days post stimulation) were added to each well. Plates were centrifuged at 2000 g for 90 minutes at 32° C.

T2 cell Transduction: T2 cells were transduced in non-tissue culture treated 6 well plates. For single PresentER construct transductions, 1 mL of virus was added to each well with 1 mL T2 cells at 1-2e6 cells/mL. For PresentER library transductions, virus was first titered and the volume of virus needed to achieve ⅓ max transduction was calculated. This volume of virus was added to each well, 2e6 T2 cells were added, and the final volume was brought to 2 mL for each well. Enough T2 cells were transduced to ensure 1000× library coverage post transduction. For all T2 cell transductions, polybrene at 0.4 μg/mL was added to each well and cells were spun at 2000 g for 2 hours at 32° C. Transduced T2 cells were selected with puromycin lug/mL and confirmed by flow cytometry.

T cell stimulation: Initial rounds of T cell stimulation were performed as described elsewhere herein. Additional rounds of T cell stimulation cells were stimulated with anti-CD2 LT2 (Miltenyi Biotec #130-093-376) and CD28 antibody 15E8 (Miltenyi Biotec #130-093-375) in addition to OKT3 and IL2.

CRISPR Cas9 gene knockout in primary T cells: Isolated and OKT3 stimulated T cells were washed three times with BTX Cytoporation medium T (catalog #47-0002). 3e6 T cells were resuspended in 90 μL of BTX medium. Samples were mixed with 5 μg modified sgRNA from Synthego and 5 μg modified CleanCap Cas9 mRNA (TriLink Biotechnologies #L7206). SgRNA sequences are as follows: 61:CGCCAGGCCGAGCCAGUUCC (SEQ ID NO: 22), 65: GGCGACACCCGGAACUGGCU (SEQ ID NO: 23), 70: CACCCGGAACUGGCUCGGCC (SEQ ID NO: 24), and 73: CCGGAACUGGCUCGGCCUGG (SEQ ID NO: 25). Samples were electroporated in 2 mm cuvette. Samples were immediately transferred to prewarmed RPMI media with IL2.

CD8 knock-out with CRISPR Cas9: Isolated and OKT3 stimulated CD4 negative T cells were washed three times with BTX Cytoporation medium T (catalog #47-0002). 3e6 T cells were resuspended in 90 μL of BTX medium. Samples were mixed with 5 μg modified sgRNA from Synthego and 5 μg modified Trilink CleanCap Cas9 mRNA (catalog #L7206). Samples were electroporated in 2 mm cuvette. Samples were immediately transferred to prewarmed RPMI media with IL2.

CD8 beta knock-out with CRISPR Cas9: sgRNAs to knock out CD8β were ordered from Synthego (CRIPSRevolution sgRNA EZ Kit). Sequences: guide sgRNA 3: ucaguaacaugcgcaucuac (SEQ ID NO: 41), guide sgRNA 4: ggcgcgccacgaugeggccg (SEQ ID NO: 42). The Lonza P3 Primary Cell 4D×Kit S (32 RCT) (Lonza #V4XP-3032) was used to prepare T cells for electroporation with the Lonza 4D Nucleofactor X Unit electroporator (Lonza #AAF-1003X). Briefly, 2e6 OKT3 stimulated CD4 negative T cells were resuspended in 15.1 μL P3 buffer. 50 pM sgRNA (Synthego) and 20 pM CleanCap Cas9 mRNA (TriLink Biotechnologies #L-2706) were mixed together with the T cells. For electroporation program EC115 was chosen. After electroporation cells were resuspended in 1 mL of RPMI containing 100 IU/mL of IL-2.

Flow Cytometry: Samples were washed with FACS buffer. Samples were stained for 30 minutes on ice, protected from light. Exemplary antibodies utilized included: anti-CD3 FITC (eBioscience #11-0037-42) stained at 1:500; Anti-CD4 FITC (eBioscience #11-0048-42) stained at 1:400 or 1:500; Anti-CD8 APC (BioLegend #344722) stained at 1:500; APC conjugated NYESO-1/A02:01 tetramer provided by the NIH; stained at 1:100 to 1:400; Anti-Vb13.1 FITC clone H131 stained at 1:50 (Biolegend #362404); Anti CD8α-APC stained at 1:400 (Invitrogen #17-0088-42); Anti-CD8B-PE stained at 1:400 (Invitrogen #12-5273-42). Samples were washed and analyzed with Guava easyCyte 11HT flow cytometer (Millipore Sigma #0500-4020). For flow assays with preincubation with anti-CD8 (3B5) (Thermo scientific #MHCD0800) or anti-CD3 (OKT3) (Miltenyi Biotec #130-093-387) antibody, antibody was added at 5 μg/mL for 30 minutes on ice, prior to being washed with FACS buffer and used for further staining.

Viral Production: For viral production of 1G4 and single minigene PresentER constructs, HEK-Ampho cells were plated and grown to ˜50% confluency on 10 cm round tissue culture plates prior to transfection with plasmids. 12.5 μg of vector and 2.5 μg of pCL-ampho packaging vector were mixed with 45 μg PEI (stock at lug/μL) in 1 mL optimum and allowed to sit for 15 minutes prior to being added dropwise to the HEK-Ampho cells. Virus was harvested at 2-3 timepoints between 24 and 72 hours post HEK-Ampho transfection. For viral production of PresentER libraries, HEK-Ampho cells were plated and grown to ˜50% confluency on 4×15 cm round tissue culture plates prior to transfection with plasmids. For each plate, 18.75 μg of library vector and 6.25 μg of pCL-ampho packaging vector were mixed with 75 μg PEI (stock at lug/μL) in 2 mL optimum and allowed to sit for 15 minutes prior to being added dropwise to the HEK-Ampho cells. Virus was harvested as described elsewhere herein.

Generation of Mutant CD8α 1G4 constructs: The 1G4 backbone was digested with SalI HF enzyme followed by CIP enzyme. Digested backbone was purified via gel extraction. An IRES site was PCR amplified from the PresentER backbone with the following primers: CAGAGGCTAGAATTCTGCAGACGTTACTGGCCGAAGCC (SEQ ID NO: 51) and CATGGTGGCTATTATCATCGTGTTTTTCAAAGGAAAACC (SEQ ID NO: 52). Amplicons were PCR purified. CD8α expression vector was purchased from Addgene (Addgene plasmid #86050). This plasmid was mutated via Q5 mutagenesis to create the M2 and M5 CD8α mutants. The following primers were used for mutant M2: CCAGGTGCTGGGTTCCAACCCGAC (SEQ ID NO: 53) and CACTTCAGCTCCACTGTC (SEQ ID NO: 54). The following primers were used for mutant M5: GCCCACTGCGGCCGAGGGGCTGGAC (SEQ ID NO: 55) and TTGTTATGGGAGAGGTATAGGAGGAAGGTGGGACTG (SEQ ID NO: 56). CD8α wildtype and mutant constructs were amplified from the plasmid backbone with the following primers: GATGATAATAGCCACCATGGCCTTACCAG (SEQ ID NO: 57) and

ATCCCGGGCCCGCGGTACCGTTAGACGTATCTCGCCGAAAGG (SEQ ID NO: 58). Amplicons were PCR purified. Final constructs were assembled via Gibson assembly using the digested 1G4 backbone, IRES amplicon and CD8α amplicons.

Luciferase Cytotoxicity Assays: GFP/Luc positive T2 cells were pulsed with synthetically generated peptides, ordered from Genemed Synthesis Inc., for 2 hours prior to being mixed 1:1 with T cells. Cells were at 0.5-1e6 cells/mL. Cells were plated in triplicate in a 96 well flat bottom plate, with opaque sides. Each well had 200 μL final volume. 5 μL luciferin was added to each well and luminescence was measured at 24-48 hours. For anti-CD8 blocking assays, anti-CD8 antibody 3B5 (ThermoFisher Scientific #MHCD0800), OKT8 (ThermoFisher Scientific #14-0086-80) or BW135/80 (Miltenyi Biotec #130-125-858) was added to T cells at 5 g/mL for at least 1 hour prior to plating.

### Example 2: Rationally Designed Libraries Identify Multiple Off-Target Peptide-MHC's Reactive with the 1G4 TCR

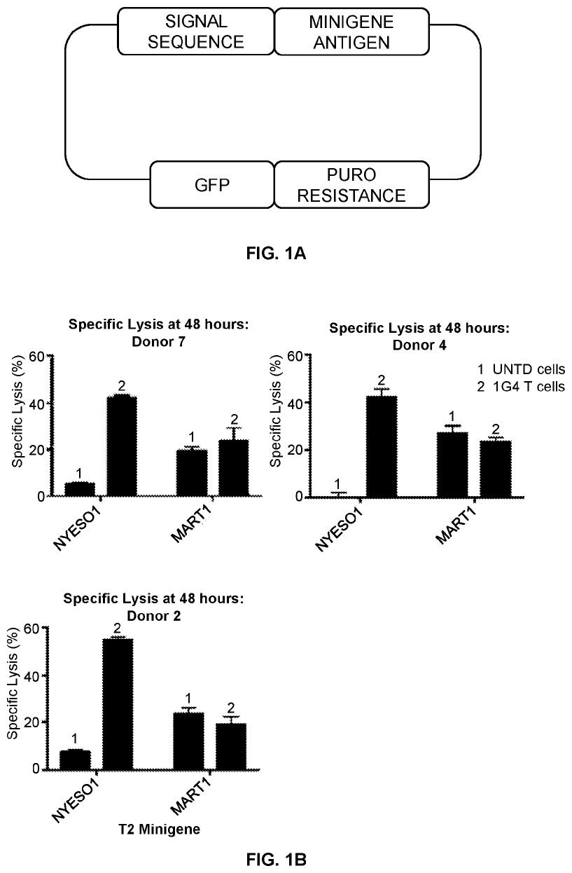

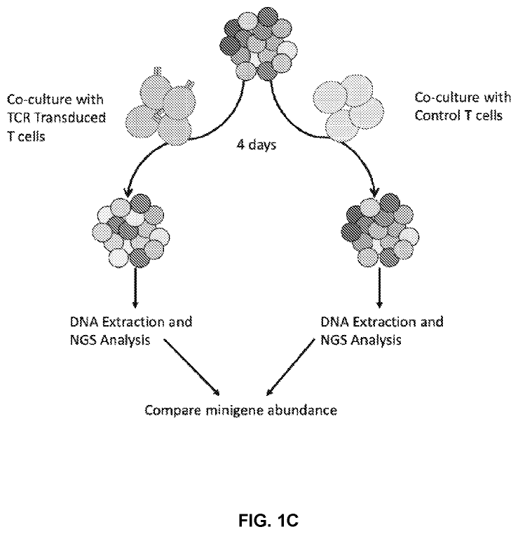

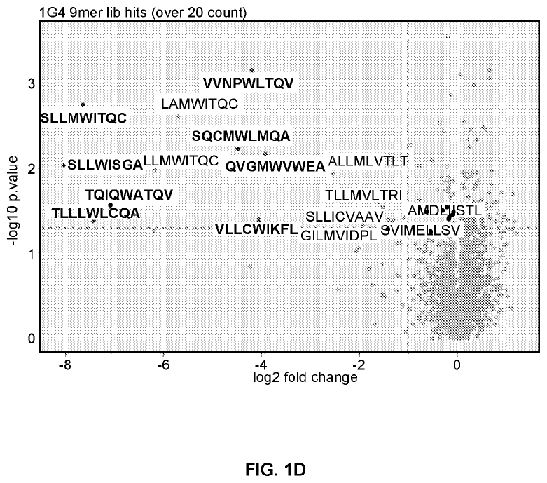

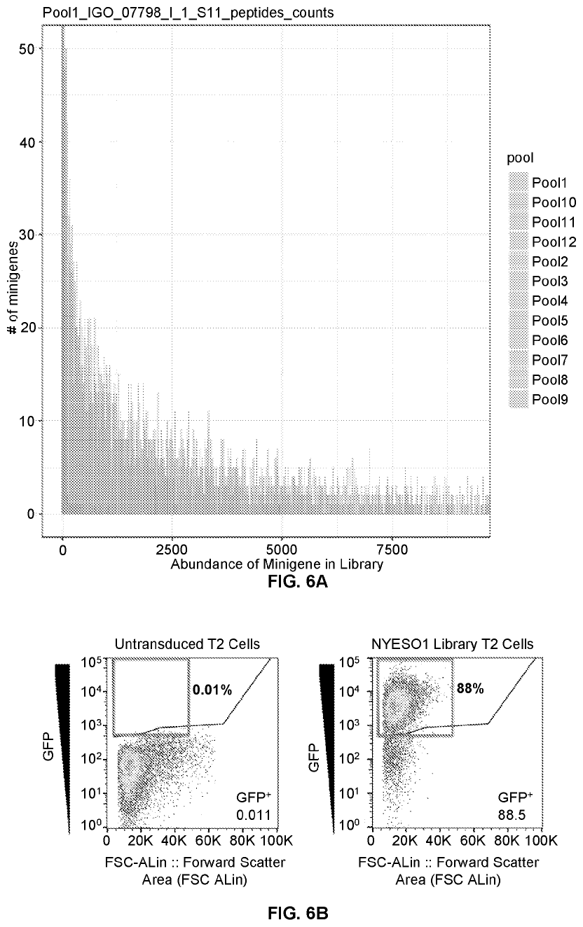

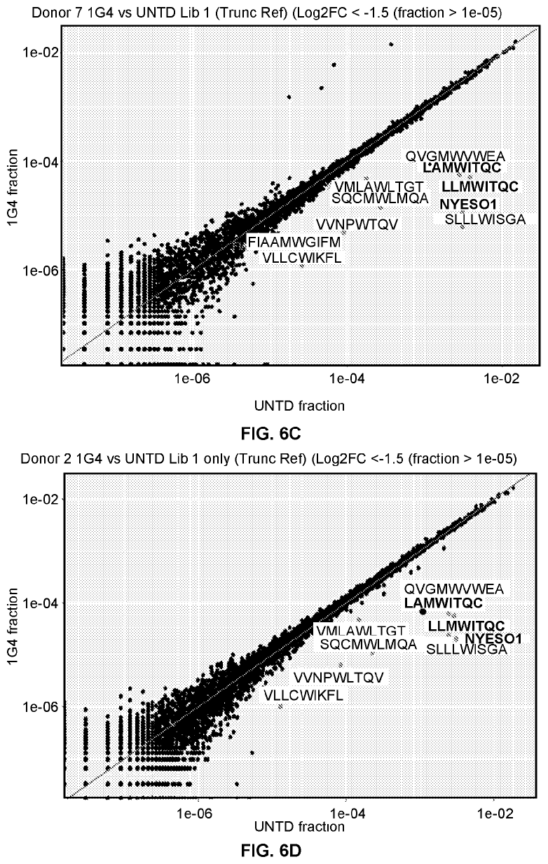

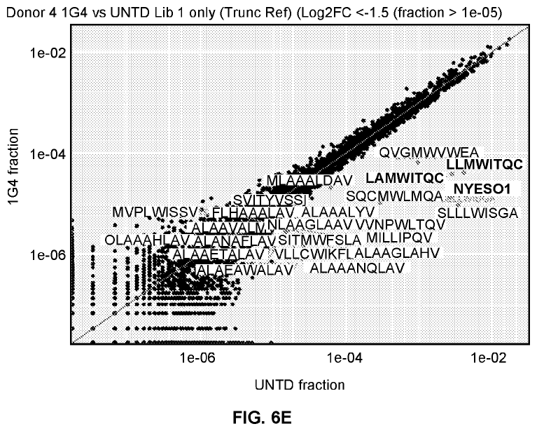

The present example describes that rationally designed libraries can identify multiple off-target peptide-MHCs reactive with a TCR (e.g., 1G4 TCR). An NY-ESO-1/A02 biased genetic expression library of peptides, derived from sequences in the human proteome, was designed to identify potentially clinically relevant off-target peptides reactive with an exemplary TCR, 1G4 TCR. The exemplary library was designed by scoring all possible 9mer peptides from the human proteome for binding to HLA-A02 using NetMHC (see, e.g., Jurtz. V et al., NetMHCpan-4.0: Improved Peptide-MHC Class I Interaction Predictions Integrating Eluted Ligand and Peptide Binding Affinity Data. J Immunol. 2017 Nov. 1; 199(9):3360-3368.). Peptides with a predicted binding affinity of under 500 nM were scored for similarity to the NY-ESO-1 peptide using the peptide: MHC binding energy covariance (PMBEC) matrix (see, e.g., Kim Y et al., Derivation of an amino acid similarity matrix for peptide: MHC binding and its application as a Bayesian prior. BMC Bioinformatics (2009) 10:394). The approximately 6,000 peptides with the highest similarly scores were included in the library. All approximately 6,000 peptides were cloned as minigenes into the PresentER plasmid, downstream of an endoplasmic reticulum (ER) signaling sequence (FIG. 1A), to facilitate peptide loading onto MHC I complexes independent of the transporter associated with antigen presentation (TAP). The exemplary PresentER plasmid used here comprises a nucleotide sequence encoding a Green Fluorescent Protein (GFP) marker, allowing for detection of transduced cells by flow cytometry. Cloning of the NY-ESO-1 based library was validated by illumina next generation sequencing (NGS) (FIG. 6A). The exemplary library was expressed in TAP deficient, HLA-A02 positive, human cell line, T2 (ATCC® CRL-1992™) (FIG. 6B). Without wishing to be bound by any one theory, use of a TAP-deficient cell line promotes presentation of minigene encoded peptides by minimizing competition for A02 binding from other cellular peptides. Approximately 3,000 of the peptides expressed in the exemplary library were transduced and represented in the T2 cells.

To minimize cross reactivity from endogenous TCRs with peptides expressed in the exemplary library, T cells from HLA-A02 negative human donors were utilized. T cells from three different donors were transduced with 1G4 TCR. To determine if 1G4 TCR expressing T cells killed their NY-ESO-1/HLA-A02 target, 1G4 TCR or untransduced (UNTD) T cells were mixed with T2 cells expressing either NY-ESO-1 (positive control) or MART-1 (AAGIGILTV, SEQ ID NO: 26) peptide by use of the PresentER system (FIG. 1B). Target cells, expressing either NY-ESO-1 or MART-1, were GFP positive and cytotoxicity was determined by flow cytometry. For all three donors utilized, specific killing of NY-ESO-1 expressing T2 cells was observed by 1G4 TCR expressing T cells, but not UNTD T cells. No significant killing of MART-1 expressing T2 cells over background was observed.

To identify 1G4 TCR off-target peptides, 1G4 TCR or UNTD T cells from each donor were mixed 1:1 with about 10 million T2 cells expressing the NY-ESO-1 biased PresentER library described above. Use of about 10 million T2 cells increases likelihood of obtaining a library coverage depth of >1000×. T2 and T cells were incubated together for about four days, distributed across 96-well round bottom plates. After incubation, the cells were harvested, DNA was extracted, and PresentER minigene coding regions were amplified by PCR for illumina NGS (FIG. 1C). This method, among other things, identifies off-target peptides in a functionally and/or clinically relevant manner (e.g., epitope targets that trigger TCR directed T cell mediated cytotoxicity are identified).

The fraction of each minigene in the 1G4 T cell screen was compared to the fraction in the UNTD T cell screen in donor matched samples (FIGS. 6C-6E). For all three donors, the positive control peptide, NY-ESO-1, along with two 8mer peptide variations, LLMWITQC (SEQ ID NO: 1), which is missing a serine at position 1 relative to NY-ESO-1 peptide, and LAMWITQC (SEQ ID NO: 2), which is missing a serine at position 1 and a leucine to alanine switch placed in position 2 relative to NY-ESO-1 peptide, were depleted. For each peptide with counts over 20 in the UNTD T cell screen, the average log 2 fold change (log 2FC) across all three donors and p-value, using a paired T-test, was calculated. Sixteen peptides were identified with a log 2FC less than −1 and a p-value of less than 0.05 (FIG. 1D, peptides labeled). Interestingly, most of the peptides with the lowest log 2FC values comprised a tryptophan (W) at position 5. Peptides with significant p-values are denoted in bold text (FIG. 1D). An additional two peptides with a tryptophan at position 5, ELSDWIHQL (SEQ ID NO: 27) and LVMQWLGQI (SEQ ID NO: 28), did not meet statistical significance, but also were robustly depleted, with average log 2FCs of −6.2 and −4.2 respectively (FIG. 8), are denoted in dark red (FIG. 1D). The motif comprising a tryptophan at position 5 was reported previously (Karapetyan A R et al., TCR Fingerprinting and Off-Target Peptide Identification. Front Immunol (2019) 10:2501.), though with a more limited set of peptides.

### Example 3: Confirmation of On-Target Cytotoxic Ability of the 1G4 T Cells by Single-Minigene Coculture Assays

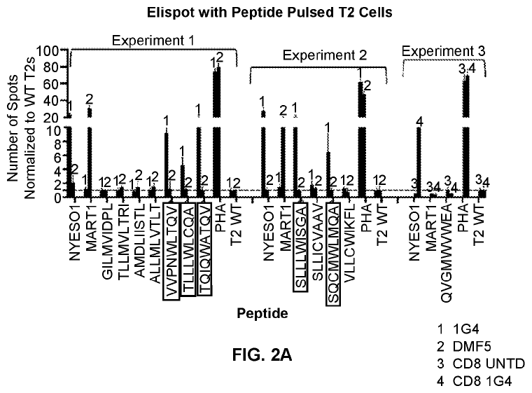

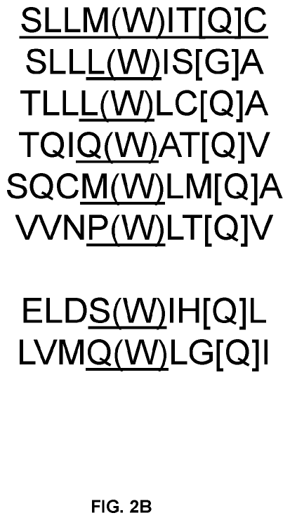

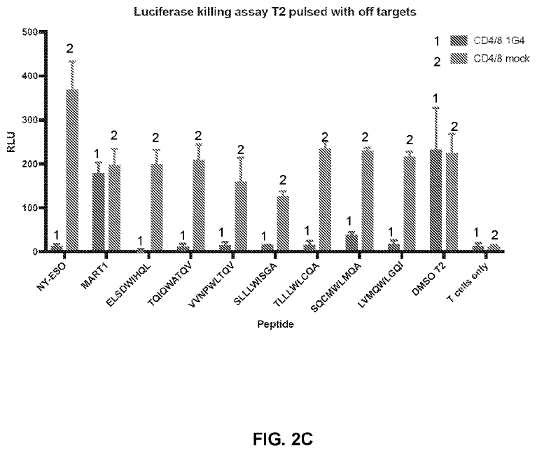

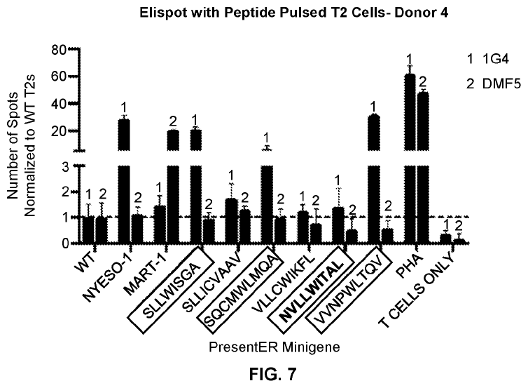

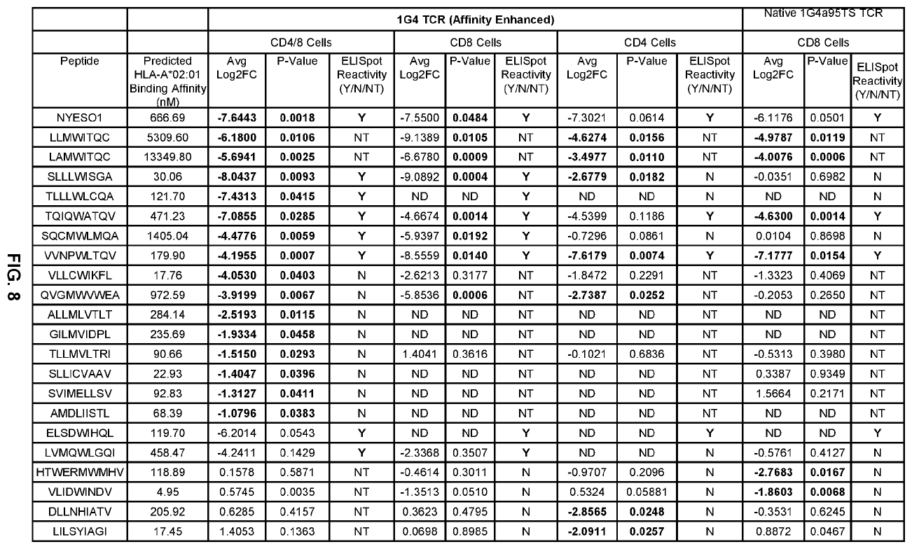

The present example describes confirmation of the ability of 1G4 TCR expressing T cells to induce on-target cytotoxicity in single-minigene coculture assays. Peptide hits were confirmed by enzyme linked immunosorobent spot (ELISpot) assay for interferon gamma (IFNγ) and cocolture killing assays where synthesized peptides were pulsed at high concentrations (e.g., 20 μg/mL) onto T2 cells or GFP/Luc T2 cells (FIGS. 2A-2B). All seven peptides with which 1G4 TCR expressing T cells reacted in these assays comprised a tryptophan at position 5 and either a glutamine (Q) or glycine (G) at position 8 (FIG. 2C). A reactive motif of the 1G4 TCR has been previously shown to include a position 5 tryptophan and a position 8 glutamine or glycine using x-scans (Karapetyan A R et al., TCR Fingerprinting and Off-Target Peptide Identification. Front Immunol (2019) 10:2501.). However, the present disclosure, among other things, identified some peptides as hits that comprise amino acids in in unexpected positions, not previously reported. Of note, VVNPWLTQV (SEQ ID NO: 29) comprises a position 4 proline (P), which was not previously reported as reactive (Karapetyan A R et al., TCR Fingerprinting and Off-Target Peptide Identification. Front Immunol (2019) 10:2501.). Additionally, the peptide NVLLWITAL (SEQ ID NO: 3), was reported as reactive with 1G4 TCR when measured by CD25 expression (Karapetyan A R et al., TCR Fingerprinting and Off-Target Peptide Identification. Front Immunol (2019) 10:2501.). Although this peptide was included in the exemplary screen described herein, it did not drop out and therefore was not identified as a functional hit. To determine if this was a false negative in the assay, potentially due to a lack of proper peptide loading and presentation through the PresentER system, peptide reactivity was assessed via ELISpot assay in which the synthesized peptide was loaded onto T2 target cells. In this assay, the peptide also did not appear as reactive (FIG. 7).

### Example 4: Cross Reactivity is not Limited to Affinity Enhanced TORs

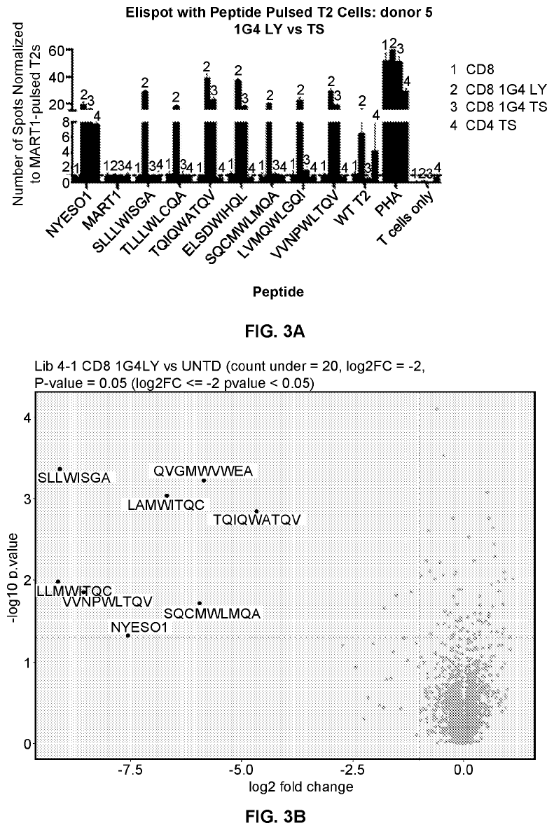

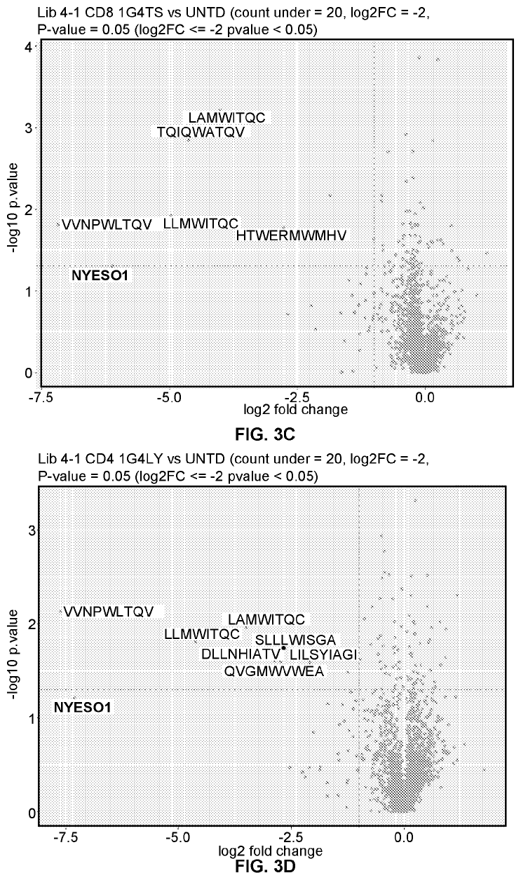

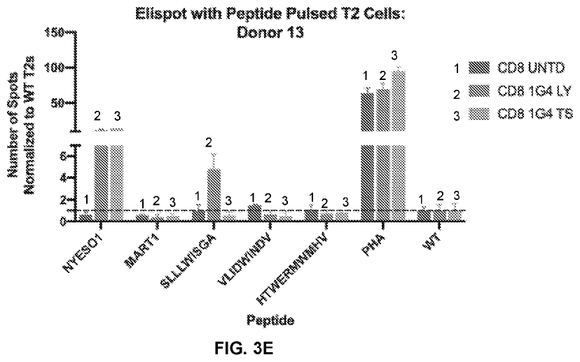

The present example describes that cross reactivity is not limited to Affinity Enhanced TCRs. Affinity Enhanced TCRs can have an increased risk of off-target reactivities. Without wishing to be bound by any one theory, it has been proposed that natural TCRs (e.g., non-Affinity Enhanced TCRs) have a higher risk of cross reactivity with self-peptides, as affinity enhancement occurs outside of the thymus and therefore bypasses thymic selection. To determine if natural TCRs also have off-target peptides from proteins found in the human proteome or if such off-targets are mainly limited to Affinity Enhanced TCRs, cross reactivities of the non-affinity enhanced, native 1G4-α95: TS TCR was assessed. Peptides previously identified as cross reactive with Affinity Enhanced 1G4 TCR were assessed for cross reactivity via ELISpot assay using T2 cells loaded with synthetic peptide. Of seven off-target peptides identified (FIG. 2B), three peptides, TQIQWATQV (SEQ ID NO: 30), ELSDWIHQL (SEQ ID NO: 27), and VVNPWLTQV (SEQ ID NO: 29) were identified as cross reactive with native 1G4-α95: TS (FIG. 3A). No correlation between predicted binding affinity to HLA-A02 or average log 2FC in the screens and peptides identified as cross reactive with native 1G4-α95: TS was observed (FIG. 8).

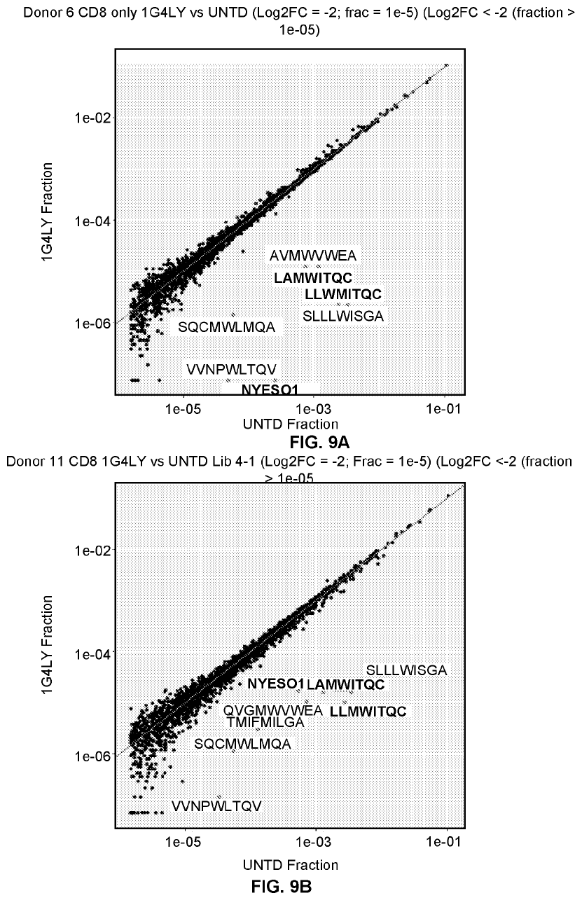

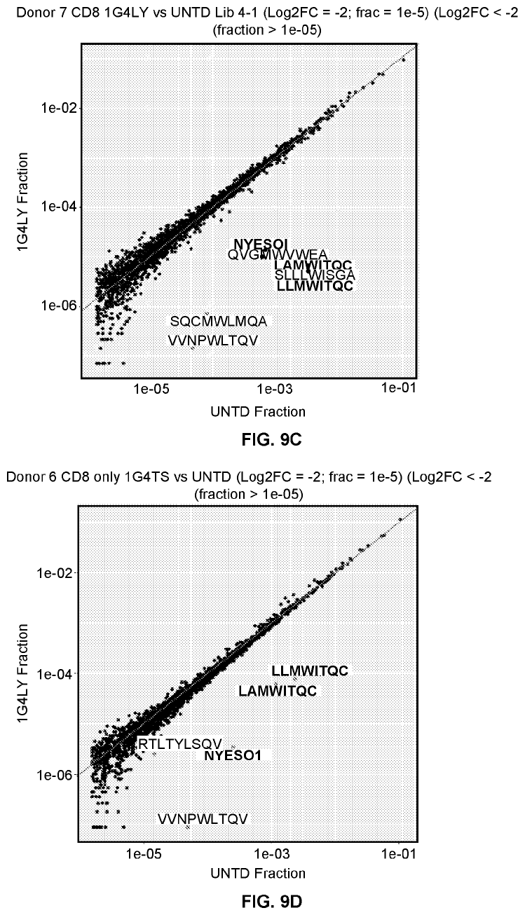

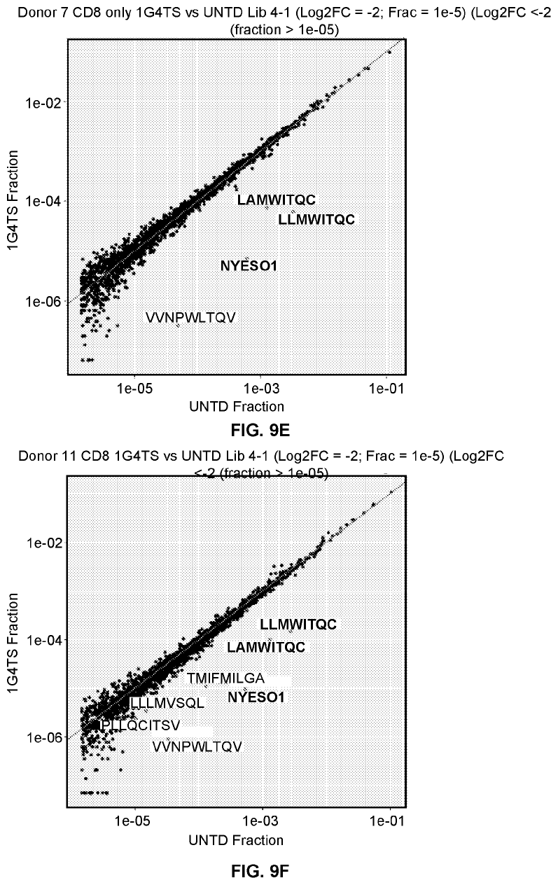

To further evaluate the cross reactivity of native 1G4-α95: TS TCR, a library screen with CD8 T cells was conducted. CD8 T cells were isolated by negative selection with anti-CD4 MACS beads and were transduced with either 1G4-α95: TS or 1G4 TCR expression constructs. Screens were performed as described previously (FIG. 1C), using T cells from three HLA-A02 negative donors. For each donor, the fractions of each peptide in the screen using CD8 TCR-T cells, either 1G4 or native 1G4-α95: TS, were compared to the screen using CD8-UNTD T cells (FIGS. 9A-9F). Screens with both CD8-1G4 and CD8-native 1G4-α95: TS cells depleted positive control peptides, NY-ESO-1, LLMWITQC (SEQ ID NO: 1) and LAMWITQC (SEQ ID NO: 2), as evident by average log 2FCs of −7.6, −9.1, and −6.7, respectively in the 1G4 screens and −6.1, −5.0, and −4.0, respectively in the native 1G4-α95: TS (FIGS. 3B-3C, FIG. 8). NY-ESO-1 expressing cells were depleted in the native 1G4-α 95: TS screens, but this depletion met a p-value of 0.0501, slightly above statistical significance (FIG. 8). In CD8-1G4 screens, there were nine additional peptides with significant p-values less than 0.05 and log 2FCs less than-1 and in CD8-native 1G4-α 95: TS there were four additional peptides (FIGS. 3B-3C), demonstrating off-targets of the 1G4 TCR were increased with affinity enhancement of the TCR. Importantly, these data demonstrated that off-target peptides derived from proteins found in the human proteome are not limited to affinity enhanced TCRs, and can be present with native TCRs.

All peptides previously identified as hits of 1G4 T cells (FIGS. 2A-2B) via coculture screens and ELISpot assay were identified as hits in screens using CD8-1G4 cells, as long as the peptides were detected by PCR (FIG. 3B, FIG. 8). Interestingly, one peptide, QVGMWVWEA (SEQ ID NO: 10), which was not previously verified by ELISpot assay (FIG. 2A), was again identified as a hit for 1G4 with an average log 2FC of −5.9 and a p-value of 0.0006. This further illustrated the difficulty in predicting off-target peptides as different assays can produce varying results.

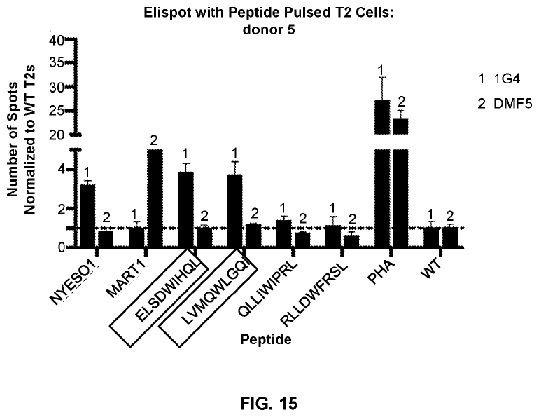

Of the peptides with P-values below 0.05 and log 2FC below −1, all five peptides, with which 1G4 T cells reacted via ELISpot assay, had a position five W and either a position 8 G or Q. In addition, peptides ELSDWIHQL (SEQ ID NO: 27) and LVMQWLGQI (SEQ ID NO: 28) also validated by ELISpot assay for IFNγ (FIG. 15).

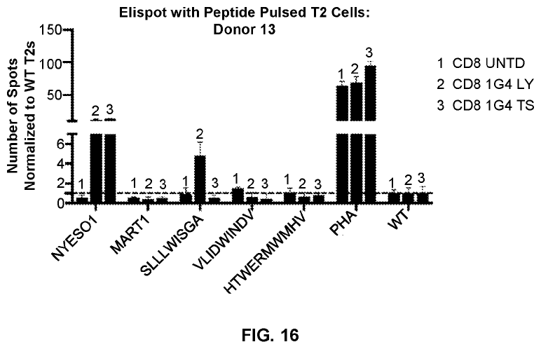

Of the off-target peptides identified by ELISpot assay for native 1G4-α 95: TS, two of the targets, TQIQWATQV (SEQ ID NO: 30) and VVNPWLTQV (SEQ ID NO: 29), were identified in the screen as hits (FIG. 3C). The third off-target ELSDWIHQL (SEQ ID NO: 27), was not detected by NGS in the library at sufficient quantities (>20 reads in the CD8-UNTD screen for all three donors) (FIG. 8). Two additional peptides were identified as possible off-targets, HTWERMWMHV (SEQ ID NO: 9) and VLIDWINDV (SEQ ID NO: 8). These peptides were further tested by ELISpot assay as previously described using synthesized peptides. Neither of these peptides were identified as reactive by ELISpot assay (FIGS. 3E and 16).

### Example 5: Cross Reactivity is Modulated by the CD8 Receptor

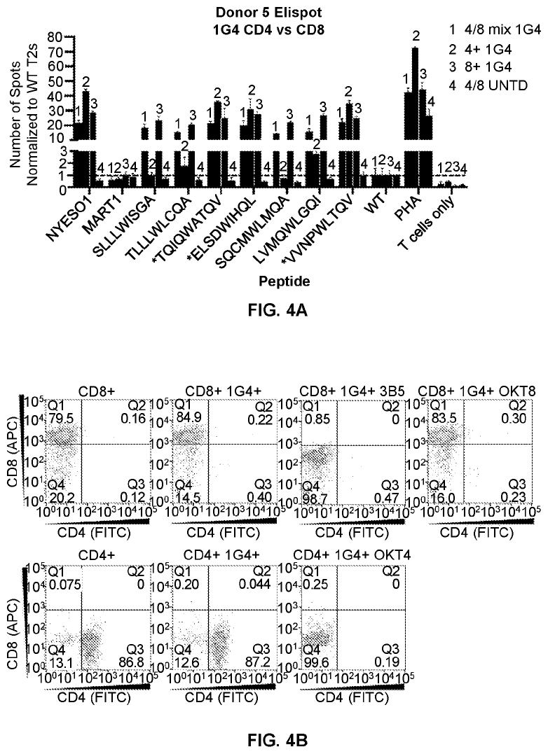

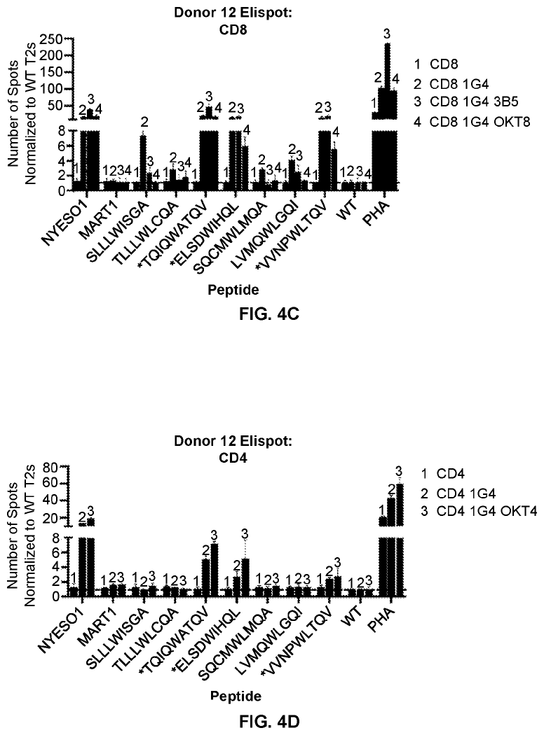

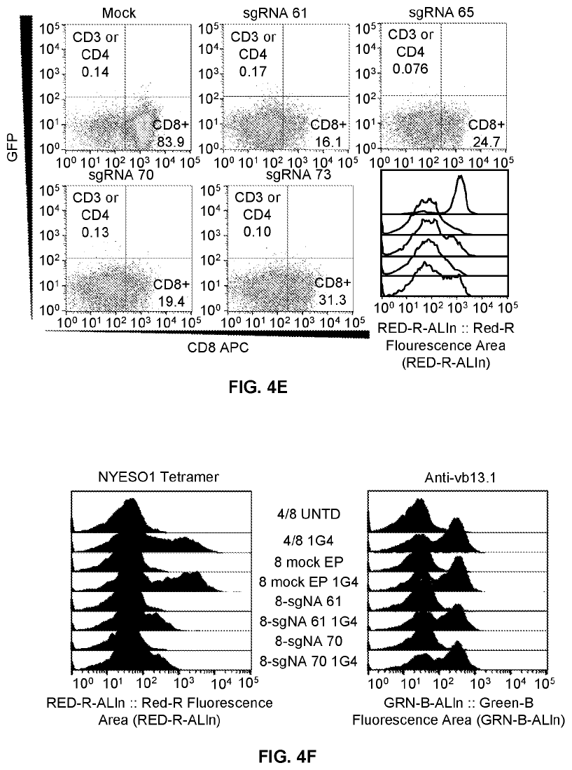

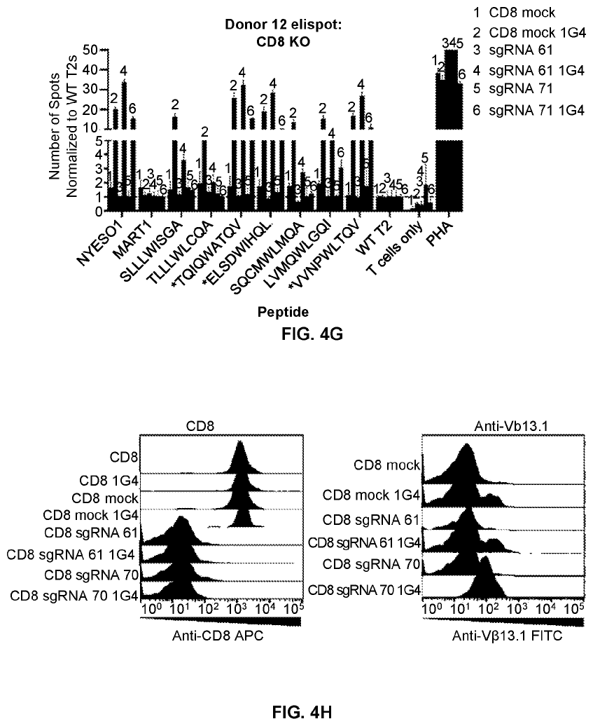

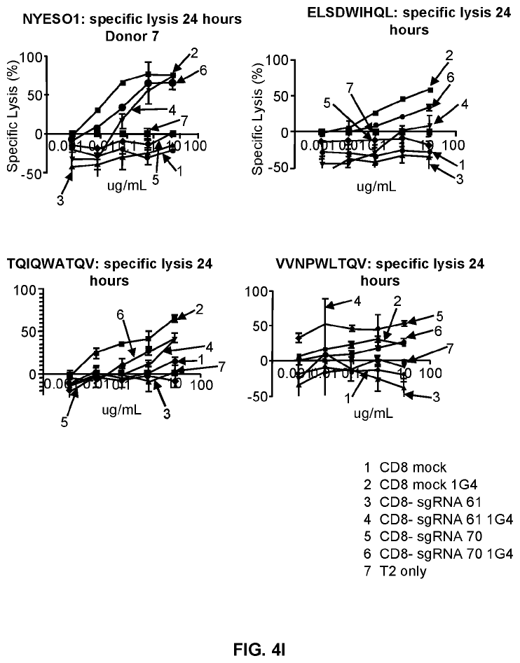

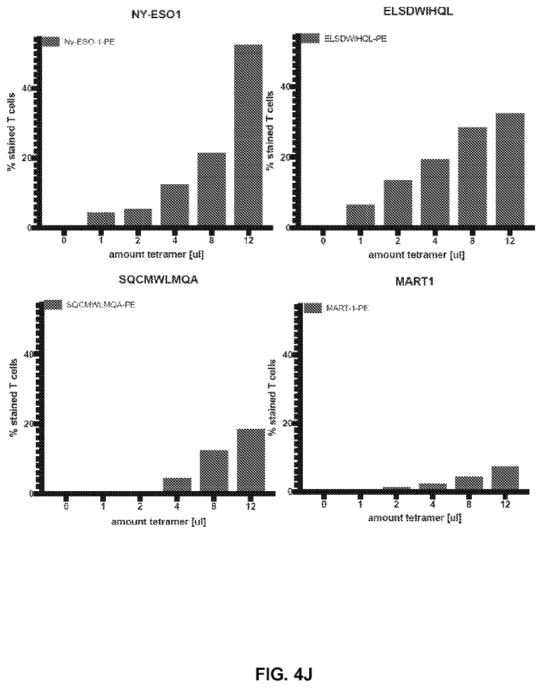

The present example demonstrates that cross reactivity is modulated by the CD8 receptor. The CD8 coreceptor can interact with MHC I on target cells to stabilize the TCR/peptide-MHC (pMHC) complex. The exemplary Affinity Enhanced 1G4 TCR utilized herein is CD8-independent (e.g., it does not rely on the CD8 coreceptor for TCR signaling upon engagement with its NY-ESO-1/HLA-A02 target). CD8-independence allows CD4-1G4 cells to also be stimulated after target engagement. However, independence can also increase the risk for off-target reactivity, especially in CD8-competent cells (Robbins P F et al., Single and Dual Amino Acid Substitutions in TCR CDRs Can Enhance Antigen-Specific T Cell Functions. J Immunol (2008) 180:6116-6131.) and, without wishing to be bound by any one theory, CD8 can stabilize additional and lower affinity TCR/pMHC interactions, further contributing to a TCR's cross-reactivity potential (Stone J D et al., Role of T Cell Receptor Affinity in the Efficacy and Specificity of Adoptive T Cell Therapies. Front Immunol (2013) 4). Therefore, differences in CD4-1G4 and CD8-1G4 reactivity were assessed by ELISpot assay using T2 cells pulsed with synthetic peptides. All peptides previously identified by the CD4/8-1G4 cells were identified as reactive with CD8-1G4-only cells (FIG. 4A). However, only three of the seven peptides previously identified, in addition to NY-ESO-1, were identified as reactive with CD4-1G4 cells: TQIQWATQV (SEQ ID NO: 30), ELSDWIHQL (SEQ ID NO: 27), and VVNPWLTQV (SEQ ID NO: 29). Therefore, the host T cell affects 1G4 TCR reactivity.

Next, the roles of CD8 and CD4 in 1G4 TCR cross reactivity were investigated using anti-CD8 and anti-CD4 antibodies, respectively. Anti-CD8 antibodies, 3B5 and OKT3, were used to block CD8 interaction with MHC I. An anti-mouse secondary antibody was used to validate anti-CD8 antibody binding (FIGS. 11A-11B). A second antibody to CD8, SK1, was also tested and its binding to T cells was blocked after prior incubation with the 3B5 antibody, indicating binding of both antibodies to sterically proximal epitopes. Interestingly, SK1 binding was not affected by preincubation with the OKT8 antibody (FIG. 4B), indicating a distinct epitope from this antibody on CD8. Despite the differences in binding, the data were consistent: after blocking with each antibody, three off-target peptides, in addition to NYESO-1, were detected by CD8-1G4: TQIQWATQV (SEQ ID NO: 30), ELSDWIHQL (SEQ ID NO: 27), and VVNPWLTQV (SEQ ID NO: 29) (FIG. 4C). Additionally, these three peptides were the same as those detected by CD4-1G4 cells (FIG. 4A).

CD4 blocking with OKT4 antibody was confirmed using a directly labeled OKT4 antibody (FIG. 4B). Binding to CD4 with anti-CD4 antibody, OKT4, had no effect on CD4-1G4 T cell cross reactivity (FIG. 4D). Taken together, these results indicated that neither CD8 nor CD4 co-receptor engagement was necessary for 1G4 cross-reactivity with NY-ESO-1, TQIQWATQV (SEQ ID NO: 30), ELSDWIHQL (SEQ ID NO: 27), or VVNPWLTQV (SEQ ID NO: 29) peptides. However, for the four peptides not recognized by CD8-blocked CD8-1G4 or CD4-1G4 cells SLLLWISGA (SEQ ID NO: 7), TLLLWLCQA (SEQ ID NO: 31), SQCMWLMQA (SEQ ID NO: 32), and LVMQWLGQI (SEQ ID NO: 28), CD8 coreceptor engagement was required for cross reactivity.

To confirm these results, cross reactivity was assessed after CD8 protein knock-out with clustered regularly interspaced short palindromic repeats (CRISPR)/CRISPR associated protein 9 (Cas9). Multiple small guide RNAs (sgRNAs) targeting CD8α were tested, as both the CD8αα homodimer and CD8αβ heterodimer are naturally stable on the cell surface, but the CD8ββ homodimer is not (Sanders S K et al., Mutations in CD8 that affect interactions with HLA class I and monoclonal anti-CD8 antibodies. J Exp Med (1991) 174:371-379; Cheroutre H et al., Doubting the TCR Coreceptor Function of CD8αα. Immunity (2008) 28:149-159.). Therefore, knock-out CD8α should eliminate all surface CD8 expression. Four sgRNAs were tested targeting CD8α, sgRNA 61, 65, 70, and 73. mRNA encoding the sgRNA and Cas9 were electroporated into CD8 T cells. CD8 expression was analyzed by flow cytometry about five days later. sgRNAs 61 and 70 diminished CD8 surface expression most efficiently, 67.8% and 64.5%, respectively, and were therefore used in future experiments (FIG. 4E). To create a completely CD8^{-/-} T cell population, CD8 MACS beads were used to remove any remaining CD8-positive cells. CD8^{-/-} 1G4 cells displayed the same cross reactivities as CD8-blocked CD8-IG4 cells and CD4-IG4 cells, as assessed by ELISpot (FIGS. 4F-4G), therefore confirming the role of CD8 in 1G4 cross reactivities by use of an orthogonal approach.

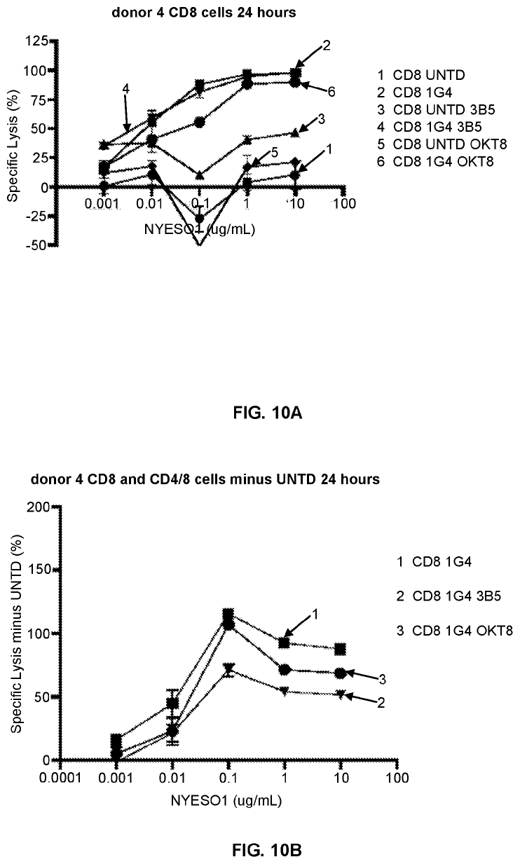

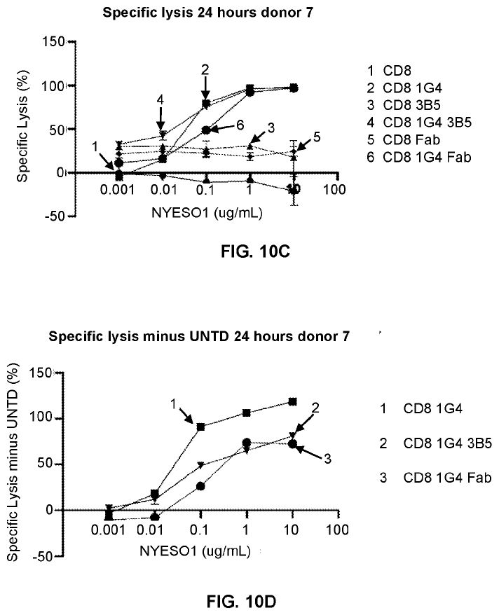

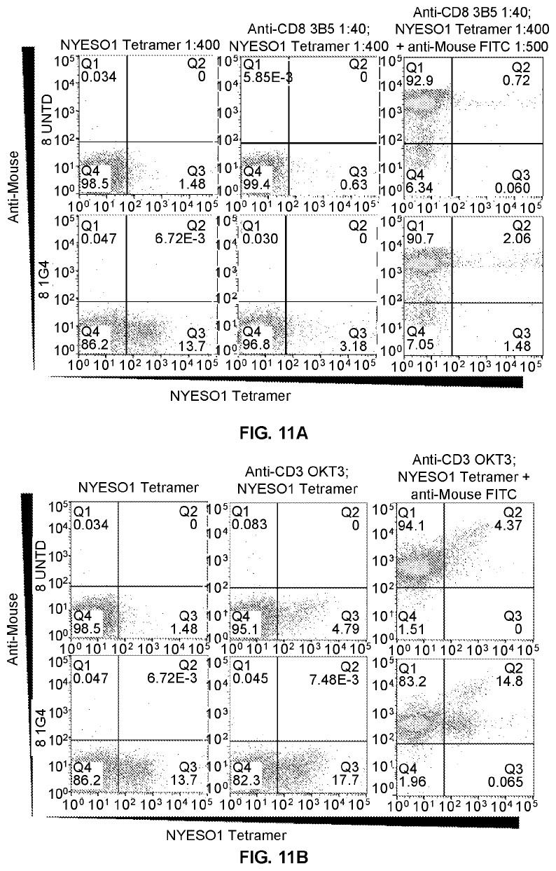

As reduction of cross reactivity of IG4 TCR may improve its specificity, but at the same time reduce its on-target efficacy, prevention of CD8 engagement and its effect on the cytotoxic abilities of these T cells was analyzed. Cytotoxicity assays were performed in GFP/Luc T2 cells, pulsed with 10-fold dilutions of the NY-ESO-1 peptide, cocultured with CD8-1G4 cells with or without anti-CD8 antibodies, 3B5 or OKT8. At 24 hours, the total killing by CD8-1G4 was the same, whether or not CD8 was blocked. However, background killing in the CD8-UNTD cells was higher in the presence of anti-CD8 antibodies, making the net cytotoxicity less (FIGS. 10A-10B). An increase in background killing and a decrease in net cytotoxicity was also observed when CD8 was blocked with an antigen binding fragment (Fab) (FIGS. 10C-10D).

One confounding explanation for these data, without wishing to be bound by any theory, is that the 1G4 transduced cells were already killing at maximum capacity whether or not CD8 is blocked, but CD8-UNTD cells were artificially stimulated by anti-CD8 antibodies and Fabs. Therefore, to further assess if blocking of CD8 did decrease overall cytotoxicity, the cytotoxic ability of CD8^{-/-} 1G4 cells was assessed. Transduction percentages were determined using the H131 antibody targeting the TCR beta chain of 1G4, Vb13.1 (FIG. 4H), as NY-ESO-1 tetramer binding was diminished in the absence of CD8, evident through diminished tetramer staining in the presence of anti-CD8 antibody, 3B5 (FIG. 11A). The three peptide hits identified as CD8-independent, VVNPWLTQV (SEQ ID NO: 29), TQIQWATQV (SEQ ID NO: 30), and ELSDWIHQL (SEQ ID NO: 27), along with the on-target peptide, NY-ESO-1, were pulsed onto GFP/Luciferase T2 cells in 10-fold serial dilutions for coculture cytotoxicity assays. For all four peptides, CD8−/− 1G4 cells killed less robustly than CD8 1G4 cells (FIG. 4I). Therefore, the beneficial effects of reductions of cross-reactivity by reducing CD8 function, were confounded by the detriment of reductions in potency.

To investigate if the ability of peptides to act as a CD8-dependent or CD8-independent off-target is based on the affinity of the 1G4 TCR for the pMHC complex, we conducted tetramer titration assays using the previously identified CD8-independent peptide, ELSDWIHQL (SEQ ID NO: 27), and the CD8-dependent peptide, SQCMWLMQA (SEQ ID NO: 32), as well as NY-ESO-1 and MART-1 peptides as positive and negative controls respectively. We saw minimal binding of the negative control MART-1/A02 tetramer at all concentrations and binding of the positive control NYESO-1/A02 tetramer at the lowest tetramer concentration. The lowest two concentrations of the ELSDWIHQL (SEQ ID NO: 27)/A02 tetramer stained 1G4 TCR transduced cells while the lowest two concentrations of the SQCMWLMQA (SEQ ID NO: 32)/A02 tetramer did not (FIG. 4J). This indicated that the ELSDWIHQL (SEQ ID NO: 27)/A02 tetramer has a higher binding affinity for the 1G4 TCR compared to the SQCMWLMQA (SEQ ID NO: 32)/A02 tetramer and could be a mechanism explaining the CD8 coreceptor requirement for SQCMWLMQA (SEQ ID NO: 32) reactivity.

Removing or blocking the CD8 coreceptor to prevent engagement with MHC I decreased off-target reactivity of the 1G4 TCR and additionally decreased reactivity with CD8-independent-peptide pulsed cells (FIG. 4I). These data prompt the hypothesis, without wishing to be bound by any one theory, that for cancer associated antigens such as the cancer germline antigen, NY-ESO-1, which are over expressed on cancer tissues relative to normal tissues, CD8 can be leveraged for TCRs that react in a CD8-independent manner to alter the therapeutic window of the TCR. Knocking out CD8 could decrease off-target potential while maintaining on-target killing if the peptide is expressed in high enough amounts on the target cells. This is in contrast to TCR-T cells directed to truly cancer specific antigens, which are very rare.

Exemplary CD8 receptor sequences utilized herein are shown in Tables 1 and 2.

TABLE 1
Exemplary CD8α nucleic acid sequences

| SEQ ID NO: | Nucleic acid sequence |
|---|---|
| SEQ ID NO: | ATGGCCTTACCAGTGACCGCCTTGCTCCTGCCGCTGGC |
| 33 | CTTGCTGCTCCACGCCGCCAGGCCGAGCCAGTTCCGGG |
| (CD8α WT) | TGTCGCCGCTGGATCGGACCTGGAACCTGGGCGAGACA |
| | GTGGAGCTGAAGTGCCAGGTGCTGCTGTCCAACCCGAC |
| | GTCGGGCTGCTCGTGGCTCTTCCAGCCGCGCGGCGCCG |
| | CCGCCAGTCCCACCTTCCTCCTATACCTCTCCCAAAAC |
| | AAGCCCAAGGCGGCCGAGGGGCTGGACACCCAGCGGTT |
| | CTCGGGCAAGAGGTTGGGGGACACCTTCGTCCTCACCC |
| | TGAGCGACTTCCGCCGAGAGAACGAGGGCTACTATTTC |
| | TGCTCGGCCCTGAGCAACTCCATCATGTACTTCAGCCA |
| | CTTCGTGCCGGTCTTCCTGCCAGCGAAGCCCACCACGA |
| | CGCCAGCGCCGCGACCACCAACACCGGCGCCCACCATC |
| | GCGTCGCAGCCCCTGTCCCTGCGCCCAGAGGCGTGCCG |
| | GCCAGCGGCGGGGGGCGCAGTGCACACGAGGGGGCTGG |
| | ACTTCGCCTGTGATATCTACATCTGGGCGCCCTTGGCC |
| | GGGACTTGTGGGGTCCTTCTCCTGTCACTGGTTATCAC |
| | CCTTTACTGCAACCACAGGAACCGAAGACGTGTTTGCA |
| | AATGTCCCCGGCCTGTGGTCAAATCGGGAGACAAGCCC |
| | AGCCTTTCGGCGAGATACGTCTAA |
| SEQ ID NO: | ATGGCCTTACCAGTGACCGCCTTGCTCCTGCCGCTGGC |
| 34 | CTTGCTGCTCCACGCCGCCAGGCCGAGCCAGTTCCGGG |
| (CD8α M2) | TGTCGCCGCTGGATCGGACCTGGAACCTGGGCGAGACA |
| | GTGGAGCTGAAGTGCCAGGTGCTGGGTTCCAACCCGAC |
| | GTCGGGCTGCTCGTGGCTCTTCCAGCCGCGCGGCGCCG |
| | CCGCCAGTCCCACCTTCCTCCTATACCTCTCCCAAAAC |
| | AAGCCCAAGGCGGCCGAGGGGCTGGACACCCAGCGGTT |
| | CTCGGGCAAGAGGTTGGGGGACACCTTCGTCCTCACCC |
| | TGAGCGACTTCCGCCGAGAGAACGAGGGCTACTATTTC |
| | TGCTCGGCCCTGAGCAACTCCATCATGTACTTCAGCCA |
| | CTTCGTGCCGGTCTTCCTGCCAGCGAAGCCCACCACGA |
| | CGCCAGCGCCGCGACCACCAACACCGGCGCCCACCATC |
| | GCGTCGCAGCCCCTGTCCCTGCGCCCAGAGGCGTGCCG |
| | GCCAGCGGCGGGGGGCGCAGTGCACACGAGGGGGCTGG |
| | ACTTCGCCTGTGATATCTACATCTGGGCGCCCTTGGCC |
| | GGGACTTGTGGGGTCCTTCTCCTGTCACTGGTTATCAC |
| | CCTTTACTGCAACCACAGGAACCGAAGACGTGTTTGCA |
| | AATGTCCCCGGCCTGTGGTCAAATCGGGAGACAAGCCC |
| | AGCCTTTCGGCGAGATACGTCTAA |
| SEQ ID NO: | ATGGCCTTACCAGTGACCGCCTTGCTCCTGCCGCTGGC |
| 35 | CTTGCTGCTCCACGCCGCCAGGCCGAGCCAGTTCCGGG |
| (CD8α M5) | TGTCGCCGCTGGATCGGACCTGGAACCTGGGCGAGACA |
| | GTGGAGCTGAAGTGCCAGGTGCTGCTGTCCAACCCGAC |
| | GTCGGGCTGCTCGTGGCTCTTCCAGCCGCGCGGCGCCG |
| | CCGCCAGTCCCACCTTCCTCCTATACCTCTCCCATAAC |
| | AAGCCCACTGCGGCCGAGGGGCTGGACACCCAGCGGTT |
| | CTCGGGCAAGAGGTTGGGGGACACCTTCGTCCTCACCC |
| | TGAGCGACTTCCGCCGAGAGAACGAGGGCTACTATTTC |
| | TGCTCGGCCCTGAGCAACTCCATCATGTACTTCAGCCA |
| | CTTCGTGCCGGTCTTCCTGCCAGCGAAGCCCACCACGA |
| | CGCCAGCGCCGCGACCACCAACACCGGCGCCCACCATC |
| | GCGTCGCAGCCCCTGTCCCTGCGCCCAGAGGCGTGCCG |
| | GCCAGCGGCGGGGGGCGCAGTGCACACGAGGGGGCTGG |
| | ACTTCGCCTGTGATATCTACATCTGGGCGCCCTTGGCC |
| | GGGACTTGTGGGGTCCTTCTCCTGTCACTGGTTATCAC |
| | CCTTTACTGCAACCACAGGAACCGAAGACGTGTTTGCA |
| | AATGTCCCCGGCCTGTGGTCAAATCGGGAGACAAGCCC |
| | AGCCTTTCGGCGAGATACGTCTAA |

TABLE 2
Exemplary CD8α amino acid sequences

| SEQ ID | |
|---|---|
| NO: | Amino Acid Sequence |
| SEQ ID | MALPVTALLLPLALLLHAARPSQFRVSPLDRTWNLGETVEL |
| NO: 36 | KCQVLLSNPTSGCSWLFQPRGAAASPTFLLYLSQNKPKAAE |
| (CD8α | GLDTQRFSGKRLGDTFVLTLSDFRRENEGYYFCSALSNSIM |
| WT) | YFSHFVPVFLPAKPTTTPAPRPPTPAPTIASQPLSLRPEAC |
| | RPAAGGAVHTRGLDFACDIYIWAPLAGTCGVLLLSLVITLY |
| | CNHRNRRRVCKCPRPVVKSGDKPSLSARYV |
| SEQ ID | MALPVTALLLPLALLLHAARPSQFRVSPLDRTWNLGETVEL |
| NO: 37 | KCQVLGSNPTSGCSWLFQPRGAAASPTFLLYLSQNKPKAAE |
| (CD8α | GLDTQRFSGKRLGDTFVLTLSDFRRENEGYYFCSALSNSIM |
| M2) | YFSHFVPVFLPAKPTTTPAPRPPTPAPTIASQPLSLRPEAC |
| | RPAAGGAVHTRGLDFACDIYIWAPLAGTCGVLLLSLVITLY |
| | CNHRNRRRVCKCPRPVVKSGDKPSLSARYV |
| SEQ ID | MALPVTALLLPLALLLHAARPSQFRVSPLDRTWNLGETVEL |
| NO: 38 | KCQVLLSNPTSGCSWLFQPRGAAASPTFLLYLSHNKPTAAE |
| (CD8α | GLDTQRFSGKRLGDTFVLTLSDFRRENEGYYFCSALSNSIM |
| M5) | YFSHFVPVFLPAKPTTTPAPRPPTPAPTIASQPLSLRPEAC |
| | RPAAGGAVHTRGLDFACDIYIWAPLAGTCGVLLLSLVITLY |
| | CNHRNRRRVCKCPRPVVKSGDKPSLSARYV |
| SEQ ID | SQFRVSPLDRTWNLGETVELKCQVLLSNPTSGCSWLFQPRG |
| NO: 47 | AAASPTFLLYLSQNKPKAAEGLDTQRFSGKRLGDTFVLTLS |
| (CD8α | DFRRENEGYYFCSALSNSIMYFSHFVPVFLPAKPTTTPAPR |
| WT) | PPTPAPTIASQPLSLRPEACRPAAGGAVHTRGLDFACDIYI |
| | WAPLAGTCGVLLLSLVITLYCNHRNRRRVCKCPRPVVKSGD |
| | KPSLSARYV |
| SEQ ID | SQFRVSPLDRTWNLGETVELKCQVLGSNPTSGCSWLFQPRG |
| NO: 48 | AAASPTFLLYLSQNKPKAAEGLDTQRFSGKRLGDTFVLTLS |
| (CD8α | DFRRENEGYYFCSALSNSIMYFSHFVPVFLPAKPTTTPAPR |
| M2) | PPTPAPTIASQPLSLRPEACRPAAGGAVHTRGLDFACDIYI |
| | WAPLAGTCGVLLLSLVITLYCNHRNRRRVCKCPRPVVKSGD |
| | KPSLSARYV |
| SEQ ID | SQFRVSPLDRTWNLGETVELKCQVLLSNPTSGCSWLFQPRG |
| NO: 49 | AAASPTFLLYLSHNKPTAAEGLDTQRFSGKRLGDTFVLTLS |
| (CD8α | DFRRENEGYYFCSALSNSIMYFSHFVPVFLPAKPTTTPAPR |
| M5) | PPTPAPTIASQPLSLRPEACRPAAGGAVHTRGLDFACDIYI |
| | WAPLAGTCGVLLLSLVITLYCNHRNRRRVCKCPRPVVKSGD |
| | KPSLSARYV |

### Example 6: CD4-1G4 Cells have a Different Set of Off-Target Peptides than CD8-1G4 Cells

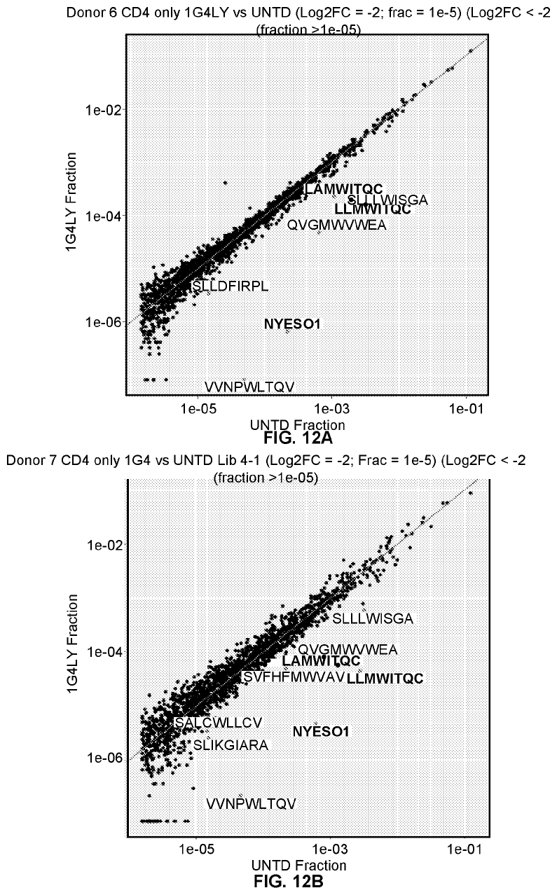

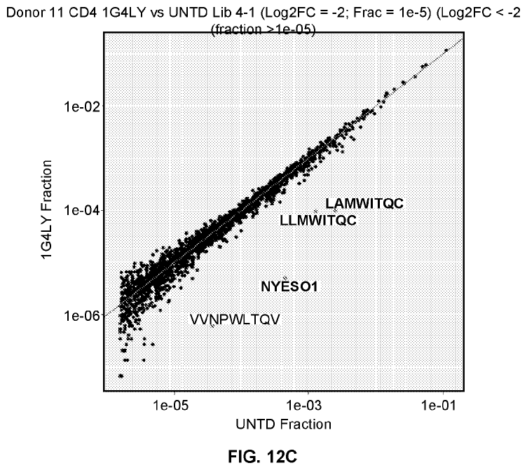

The present example demonstrates CD4-1G4 cells have a different set of off-target peptides than CD8-1G4 cells. Thus far, all identified off-targets of the 1G4 TCR have been off-targets of CD8-1G4 cells, while only three of the peptides have been identified as off-targets of CD4-1G4 cells. To assess if there are additional peptides targeted only by CD4 cells, we performed a library screen using CD4-1G4 cells (FIG. 3D, FIGS. 12A-12C). Of note, NY-ESO-1 expressing cells were depleted, with an average log 2FC of −7.3. The previously identified CD8-independent off-target of 1G4, VVNPWLTQV (SEQ ID NO: 29), was also depleted, with an average log 2FC of −7.6. Peptides SLLLWISGA (SEQ ID NO: 7) and QVGMWVWEA (SEQ ID NO: 10) were also depleted with average log 2FCs of −2.7 (FIG. 8). Peptide, SLLLWISGA (SEQ ID NO: 7) was identified as a CD8-dependent off-target of 1G4; there was no detectable reactivity over background by CD4-1G4 cells or CD8-blocked CD8-1G4 cells by ELISpot assay (FIGS. 4A and 4C). Peptide, QVGMWVWEA (SEQ ID NO: 10), was depleted in library screens using CD4/8-1G4 cells, CD8-1G4 cells alone, and CD4-1G4 cells alone, but was not a hit in other non-cytotoxicity assays. CD4-1G4 screen data for individual donors showed QVGMWVWEA (SEQ ID NO: 10) expressing cells depleted in all screens, with log 2FCs of −3.8, −3.3, and −1.2 (FIGS. 8A-8C). Therefore, QVGMWVWEA (SEQ ID NO: 10)/HLA-A02 appears to be a CD8-independent target of 1G4.

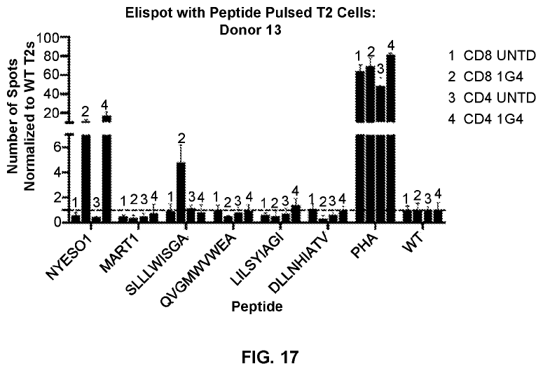

Three newly identified peptides, based on an average log 2FC less than-1 and p-value less than 0.05 also were identified as hits in screens with CD4-1G4 cells: DLLNHIATV (SEQ ID NO: 6), LILSYIAGI (SEQ ID NO: 11), and SAFEFLSSA (SEQ ID NO: 39). Peptides DLLNHIATV (SEQ ID NO: 6) and LILSYIAGI (SEQ ID NO: 11), with average log 2FC less than-2, were tested for reactivity with 4-1G4 cells by ELISpot assay. Neither of these peptides were confirmed as reactive through this method (FIG. 17). In summary, the cross-reactive peptides reactive with CD8 or CD4 T cells bearing the same TCR can differ, whereby the cross-reactive peptides of CD4 1G4 T cells are a subpool of the cross-reactive peptides of CD8 1G4 T cells.

### Example 7: PresentER Libraries Looking Forward and Back

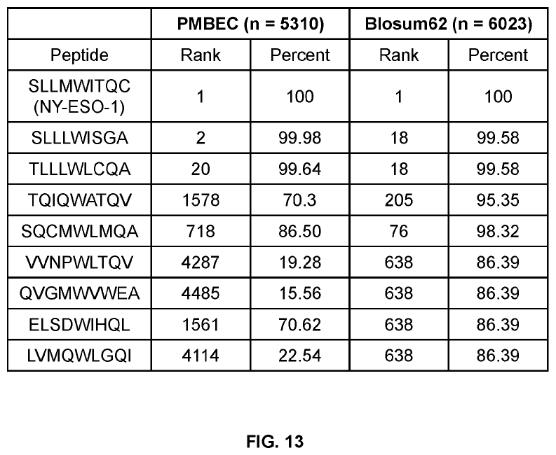

The present example demonstrates bias assessment of the exemplary PresentER libraries utilized as described herein. In an effort to understand the bias in the exemplary PresentER libraries utilized herein, and to increase the number of peptides most likely to cross-react in future library designs, use of the scoring algorithms, Blosum62 and PMBEC, to identify improved peptide sequences was assessed. One benefit of Blosum62 is that it allows comparisons of peptide similarities across different sequence lengths, by incorporated gap and extension penalties. For these analyses, a “gap penalty” of 1 and an “extension penalty” of 4 was utilized. Of the hits already identified and confirmed with ELISpot assay in the experiments described thus far, the Blosum62 algorithm scored reactive peptides more accurately than PMBEC (FIG. 13). Of note were peptides VVNPWLTQV (SEQ ID NO: 29) and LVMQWLGQI (SEQ ID NO: 28), which were at 19.28% and 22.54% (ranked at 4287 and 4114) respectively by PMBEC, but both at 86.39% (ranked at 638) by Blosum62 scoring. Additionally, peptide QVGMWVWEA (SEQ ID NO: 10), which was identified as a hit in numerous screens but did not verify by ELISpot, was at 15.56% (ranked at 4485) by PMBEC and at 86.39% (ranked at 638) by Blosum62 scoring (FIG. 13). Therefore, use of the Blosum62 scoring algorithm to score peptides for library design may be particularly useful, including, for example, for the 1G4 TCR/NY-ESO-1 peptide. However, these algorithm comparisons may be specific for the 1G4 TCR/NY-ESO-1 peptide. As additional PresentER library screens are performed, biased towards different peptides, additional analyses will need to be done to ensure the best library designs are used.

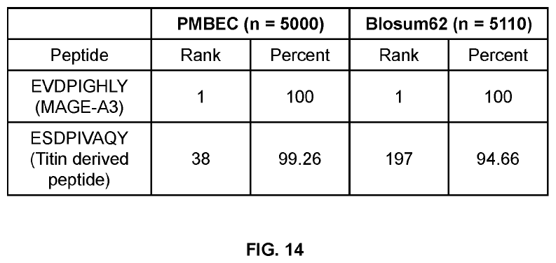

To assess if screens described herein could detect the cross-reactivity of the MAGE-A3 TCR with a peptide from titin, we designed a biased library based on the MAGE-A3 peptide, EVDPIGHLY (SEQ ID NO: 4), and HLA-A*01:01 (A01) (Linette G P et al., Cardiovascular toxicity and titin cross-reactivity of affinity-enhanced T cells in myeloma and melanoma. Blood. (2013) 122:863-871, Cameron B J et al., Identification of a titinderived HLA-A1-presented peptide as a cross-reactive target for engineered MAGE A3-directed T cells. Sci Transl Med (2013) 5:197ra103-197ra103.). Similar to how the NY-ESO-1 library was designed, all 9mer peptides in the human proteome were scored for binding to HLA-A01 by NetMHC 4.0. All peptides with binding below 500 nM were then scored for similarity to the MAGE-A3 peptide using either PMBEC or Blosum62. Both scoring methods included the titin derived peptide, ESDPIVAQY (SEQ ID NO: 5) (Cameron B J et al., Identification of a titinderived HLA-A1-presented peptide as a cross-reactive target for engineered MAGE A3-directed T cells. Sci Transl Med (2013) 5:197ra103-197ra103.) in library design (FIG. 14). PMBEC scoring placed the titin derived peptide at 99.26%, ranked at 38 out of 5000 peptides that would have been included. Blosum62 scoring with a “gap penalty” of 1 and an “extension penalty” of 4 identified the titin derived peptide 94.66%, ranked at 197 out of 5110 peptides that would have been included. Therefore, we believe that the PresentER library screening method may have been able to predict the toxicities seen with the MAGE-A3 TCR.

### Example 8: Expression of an Exogenous Mutant CD8α Chain in CD8^{-/-} 1G4 Cells Decreases Cross-Reactivity while Maintaining Specificity and Cytotoxic Potential

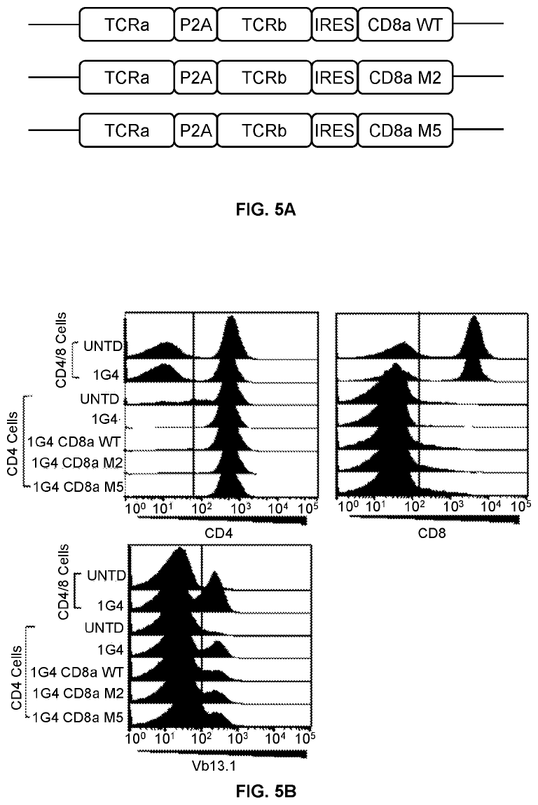

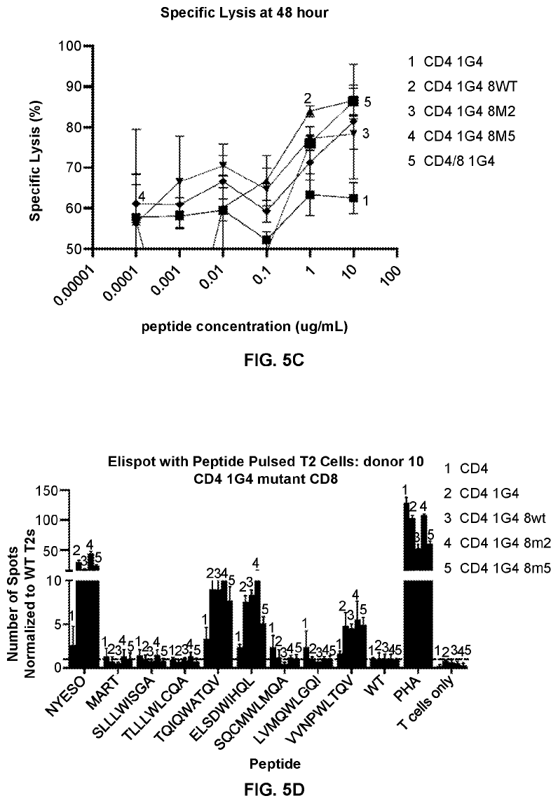

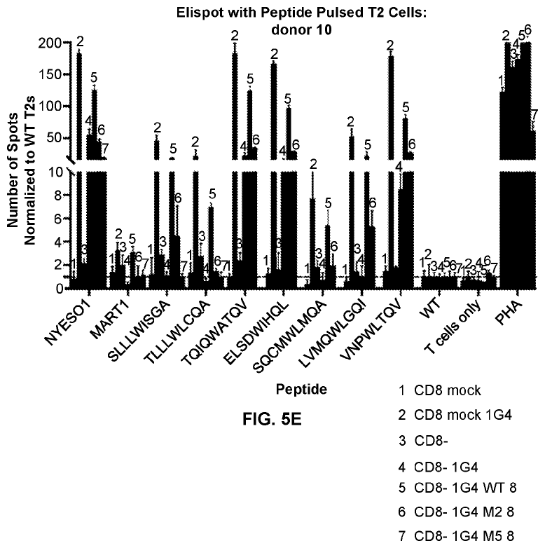

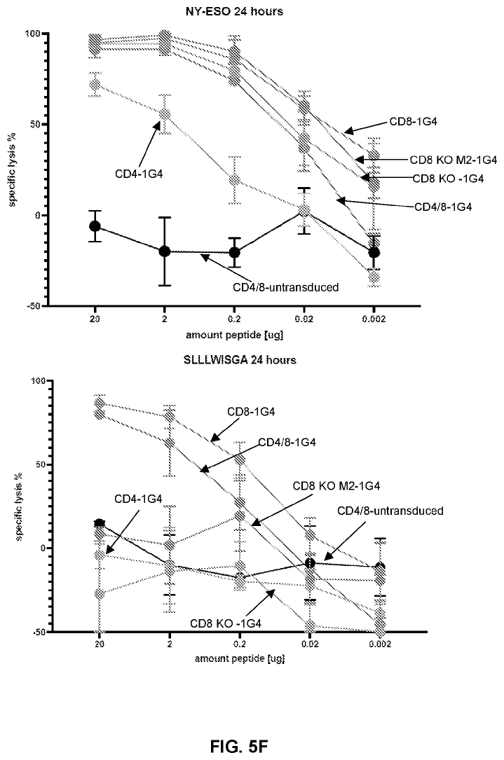

The present example demonstrates that mutant CD8αα homodimers increased cytotoxic potency of CD4 cells while maintaining specificity. Three different CD8α chains, wild type (WT) and two mutants (M2 and M5), were cloned downstream of the 1G4 TCR, separated by an internal ribosomal entry site (IRES) (FIG. 5A). The CD8α chain mutants, M2 and M5, were previously predicted to have decreased interaction with HLA class I (Sanders S K et al., Mutations in CD8 that affect interactions with HLA class I and monoclonal anti-CD8 antibodies. J Exp Med (1991) 174:371-379).

The CD8α chains can form a homodimer when expressed in CD4 cells. While CD8αα homodimers have been observed naturally, their role is poorly understood. CD4-1G4 cells expressing WT or either mutant CD8α chain, M2 or M5, had increased cytotoxicity compared to CD4-1G4 cells (FIGS. 5B-C). Importantly, there was not an increase in off-target reactivity as assayed by ELISpot, even with the CD4-1G4 CD8α WT cells (FIG. 5D). This was unexpected as there is evidence that the CD8αα homodimer can interact with HLA class I molecules. However, these data indicated that while the CD8αα homodimer increased cytotoxicity, it does not contribute to off-target reactivity in CD4 cells in the same way the CD8αβ heterodimer does in CD8 cells.

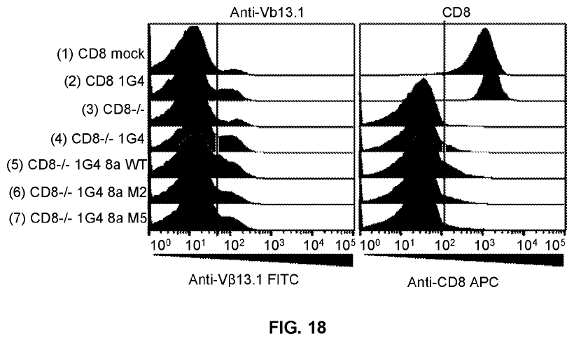

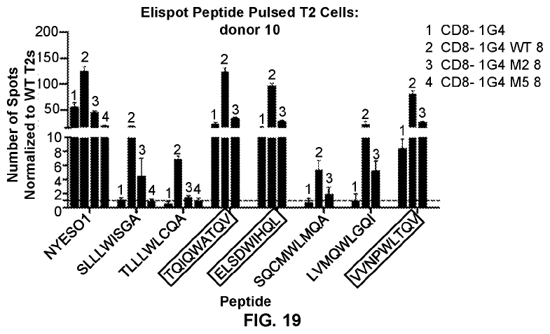

To determine if decreasing off-target reactivity while increasing cytotoxicity could be achieved in CD8 T cells, wild type and mutant CD8α with 1G4 constructs were expressed in CD8α T cells. In this context, wild type and mutant CD8α chains could dimerize with wild type CD8β chain to form a CD8αβ heterodimer. First, a population of CD8α^{-/-} T cells from HLA-A02 negative donors were generated and isolated using CRISPR/Cas9 and CD4 MACS beads, as described elsewhere herein. After four days, T cells were re-stimulated with anti-CD2/CD3/CD28 antibodies to trigger division and allow for increased retroviral transduction. Two days after re-stimulation and six days after CD8α-targeted CRISPR/Cas9, CD8^{-/-} cells were isolated by negative selection using CD8 MACS beads. Cells were then transduced with retroviral constructs encoding either 1G4 or 1G4 CD8α WT, M2, or M5 (FIG. 18). CD8^{-/-} 1G4-CD8α⁺ WT cells reacted with all peptides to which CD8^{-/-} 1G4 cells reacted. CD8^{-/-} 1G4-CD8α⁺ M2 or M5 cells only showed off-target reactivity with peptides identified as CD8-independent targets of 1G4 (FIG. 5E and FIG. 19). These findings indicated that CD8α WT with CD8β maintained CD8's interaction with HLA class I and therefore would not decrease the off-target potential of the 1G4 TCR. However, the CD8α M2 and M5 mutants in conjunction with CD8β did not contribute to cross reactivity.

We next looked to determine if expression of the mutant CD8α constructs in CD8^{-/-} cells would restore some of the cytotoxicity that is lost upon knock out of CD8α. To do this we used CRISPR/Cas9 to knock out CD8α. We next transduced the cells with constructs encoding either 1G4 or 1G4 CD8α M2. The CD8α M2 variant was used as that mutant has been reported to have the least interaction with HLAI (Sanders S K et al. J Exp Med (1991) 174:371-379). These cells were used in a T2 coculture killing assay, as described previously, using the on-target peptide NY-ESO-1 as well as the previously identified CD8-dependent peptide SLLLWISGA (SEQ ID NO: 7). The 1G4 CD8α^{-/-} as well as 1G4 CD8α^{-/-} M2 cells were able to kill T2 cells pulsed with NY-ESO-1 peptide as efficiently as CD8-G4 cells (FIG. 5F). Interestingly, the CD8α^{-/-} M2 1G4 T cells did not show cytotoxicity against the CD8 dependent off target SLLLWISGA (SEQ ID NO: 7) (FIG. 5F) confirming that exogenous mutant CD8α^{-/-} M2 in conjunction with endogenous CD8β is able to decrease off-target reactivity while maintaining on target cytotoxicity.

### Example 9: CD4 and CD8 Cells Expressing a Different TCR, DMF5, have Different Off Target Repertoires

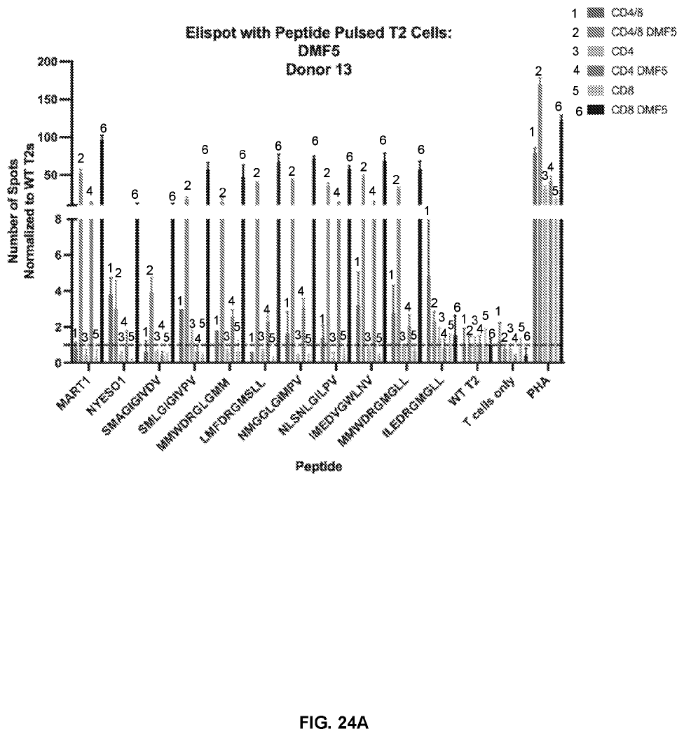

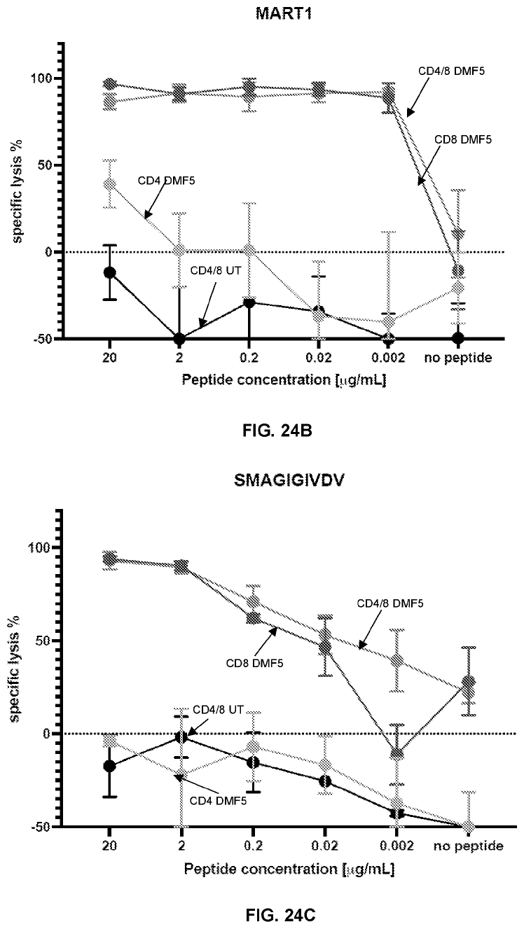

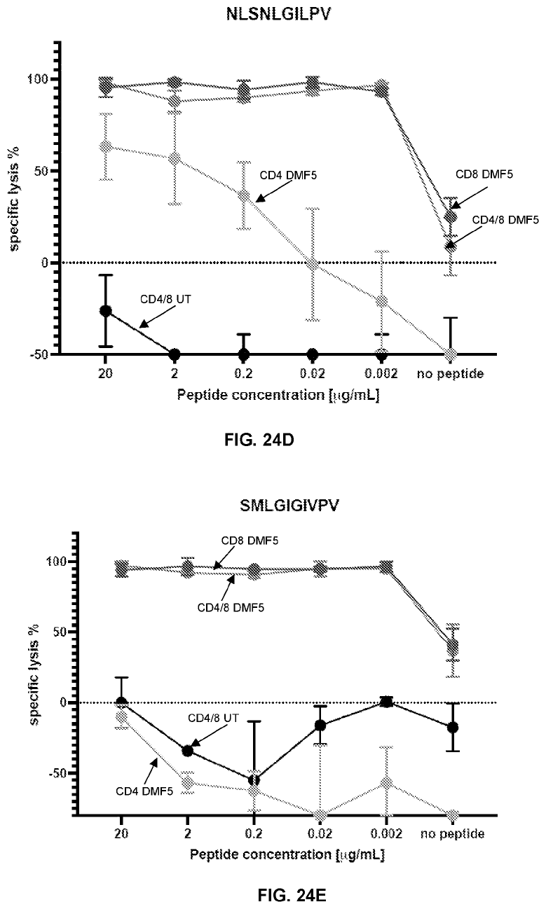

Thus far we have demonstrated that CD8 and CD4 T cells expressing the MHCI restricted TCR 1G4 have a different repertoire of off targets. We next investigated whether this phenomenon is limited to the 1G4 TCR or whether the concept is generalizable and also can be applied to other CD8-independent TCRs. The DMF5 TCR has previously been reported to be reactive in a CD8-independent manner with its MART-1 peptide target (AAGIGILTV, SEQ ID NO: 26) in the context of A02 (DOI: 10.4049/jimmunol. 177.9.6548). Numerous off-target peptides for MART-1 were previously reported (DOI: 10.1038/s41589-018-0130-4). These off targets were tested for CD8-independence. T cells were separated for CD4 or CD8 using negative selection as done previously. A mixture of CD4/CD8 cells, purified CD4 cells, or purified CD8 cells each expressing the DMF5 TCR were tested for reactivity with MART-1 peptide along with the nine previously identified peptide targets via ELISpot assay for IFNγ. MART-1 peptide and 8 of the previously identified peptide targets were reactive with both CD4/8-DMF5 and CD8-DMF5 cells. However, only MART-1 peptide and two additional peptides, NLSNLGILPV (SEQ ID NO: 44) and IMEDVGWLNV (SEQ ID NO: 46), were identified as reactive with CD4-DMF5 cells, making these peptides (D) 8-independent targets of the DMF5 TCR (FIG. 24A). This was confirmed by cytotoxic killing assays using the CD8 independent targets MART-1 peptide and NLSNLGILPV (SEQ ID NO: 44) and the CD8 dependent targets SMAGIGIVDV (SEQ ID NO: 43) and SMLGIGIVPV (SEQ ID NO: 45) (FIGS. 24B-24E). Indeed, CD4 DMF5 expressing T cells only showed cytotoxicity for the CD8 independent target NLSNLGILPV (SEQ ID NO: 44) and the on-target MART-1 peptide itself, though with less potency than CD8 DMF5.

### Example 10: Eliminating Formation of CD8β in TOR Transduced T Cells Results in Reduced Off-Target Reactivity

The present example demonstrates that by eliminating the formation of CD8β in TCR transduced T cells, the off-target reactivity to peptides can be reduced, while on-target reactivity is maintained.

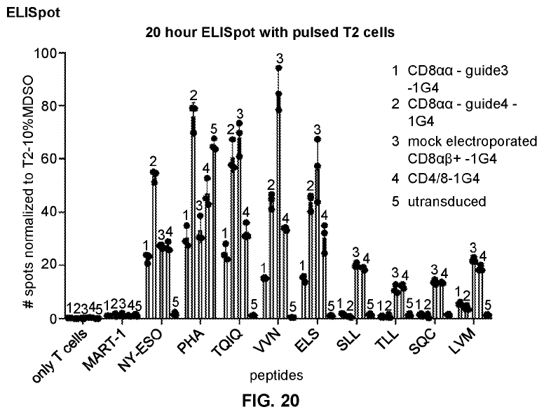

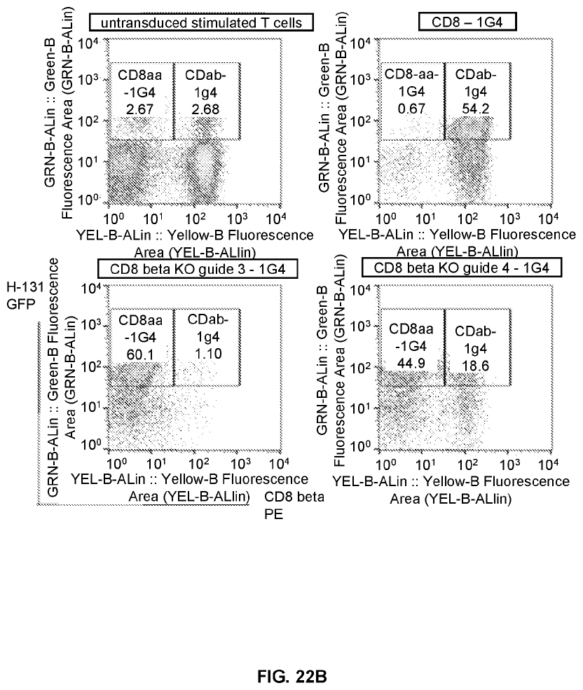

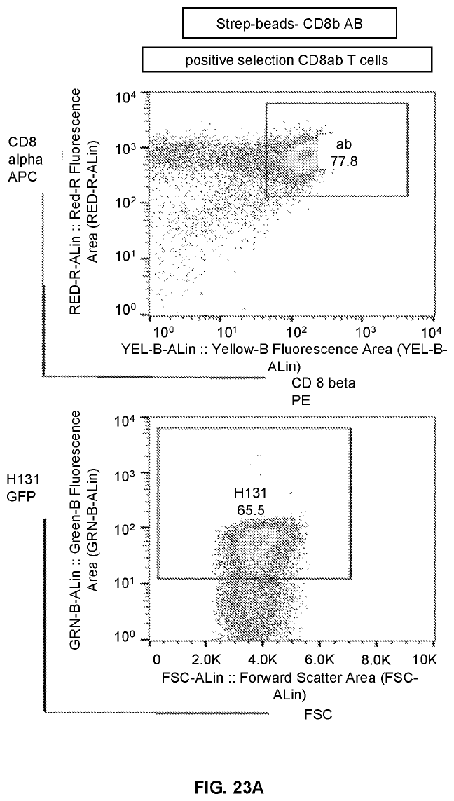

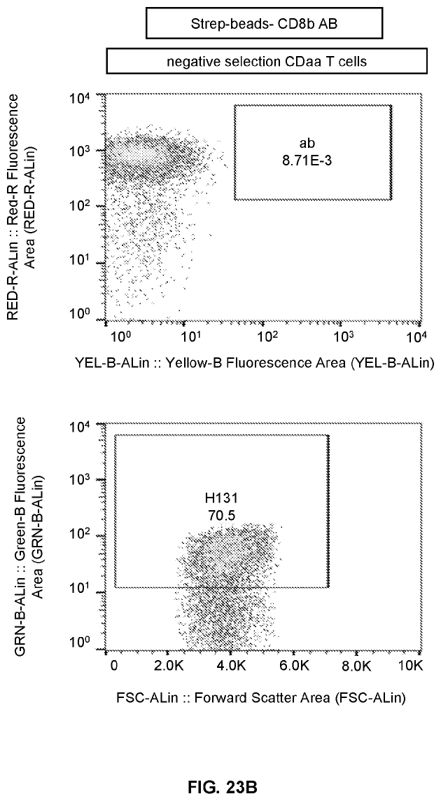

All peptides previously identified as off-targets of the CD4/CD8^{-/-} 1G4 cells by ELISpot assay using T2 cells pulsed with synthetic targets and CD8β−/− CD8αα homodimer expressing CD4 negative 1G4 T cells were tested (FIG. 20). Two different CRISPR sgRNAs to achieve the knockout of CD8β, guide 3 and guide 4, were utilized (FIGS. 22A-22B). Use of guide 4 resulted in a knockout efficiency of 72% and therefore the cells were further processed for negative MACS separation using an antibody against CD8β, resulting in a population of >99% CD8β negative T cells (FIGS. 23A-23B). Guide 3 resulted in a knockout efficiency of >95% and these cells therefore were not further separated by MACS.

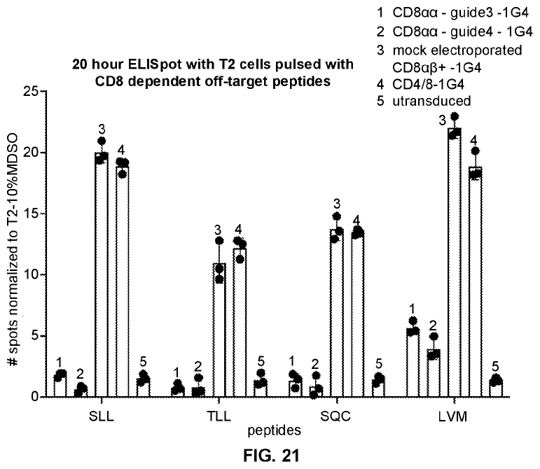

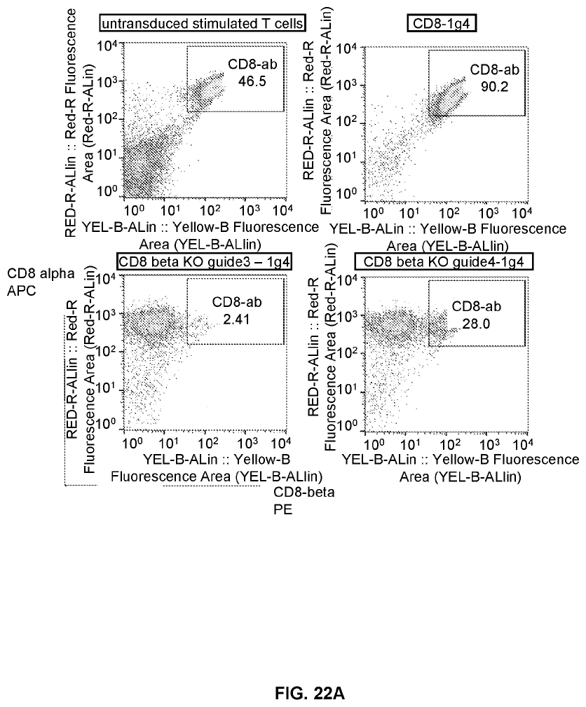

All peptides previously identified as CD8-dependent showed no reactivity with CD8αα-1G4-T-cells (FIG. 21). Interestingly, the CD8αα 1G4 T cells not purified by MACS showed slightly more reactivity to the CD8-dependent off-targets than the MACS separated CD8αα-1G4-T cells, indicating that even a small percentage of CD8αβ heterodimer expressing T cells (<5%) is enough to elicit reactivity to peptides, whereas the reactivity in purified CD8αα expressing T cells is virtually eliminated. It is important to note that the reactivity to the on-target peptide SLLMWITQC (SEQ ID NO: 40) in these cells did not significantly change, compared to CD8αβ expressing mock electroporated 1G4 transduced T cells.

Thus, eliminating the formation of CD8β in TCR transduced T cells, the off-target reactivity to peptides can be reduced, while at the same time the on-target reactivity is maintained. Surprisingly, these studies reveal that a change in TCR reactivity can be made by modulating TCR co-receptors without altering the TCR itself which will have important implications for understanding the mechanisms of TCR binding and will allow modulation of TCR reactivity to reduce potential toxicity, while maintain on-target activity.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

1. An engineered cytotoxic T cell that
a. comprises a T cell receptor (TCR) that binds to a target antigen and/or a nucleic acid encoding the T cell receptor;
b. lacks detectable expression or activity of a wild-type CD8 alpha polypeptide comprising the amino acid sequence of SEQ ID NO: 36 or SEQ ID NO: 47; and
c. comprises a non-endogenous expression vector that includes a nucleic acid sequence encoding a mutant CD8 alpha polypeptide comprising the amino acid sequence of SEQ ID NO: 37, SEQ ID NO: 38, SEQ ID NO: 48, or SEQ ID NO: 49, optionally wherein the nucleic acid sequence of the mutant CD8 alpha polypeptide includes SEQ ID NO: 34 or SEQ ID NO: 35 and/or is operably linked to an expression control sequence.

2. The engineered cytotoxic T cell of claim 1, wherein the TCR is a native TCR, a non-native TCR, or a mimic TCR or wherein the TCR is IG4, DMF5, or A6.

3. (canceled)

4. The engineered cytotoxic T cell of claim 1, wherein the mutant CD8 alpha polypeptide exhibits reduced binding to major histocompatibility complex (MHC) relative to the wild-type CD8 alpha polypeptide.

5. The engineered cytotoxic T cell of claim 1, wherein the engineered cytotoxic T cell comprises a deletion, an inversion, a missense mutation, a nonsense mutation, or a frameshift mutation in a nucleic acid sequence encoding the wild-type CD8 alpha polypeptide, optionally wherein the nucleic acid sequence encoding the wild-type CD8 alpha polypeptide is SEQ ID NO: 33.

6. The engineered cytotoxic T cell of claim 1, wherein the engineered cytotoxic T cell comprises an inhibitory nucleic acid that specifically targets and inhibits the expression of a nucleic acid sequence encoding the wild-type CD8 alpha polypeptide, optionally wherein the nucleic acid sequence encoding the wild-type CD8 alpha polypeptide is SEQ ID NO: 33 or optionally wherein the inhibitory nucleic acid is an antisense oligonucleotide, a siRNA, a sgRNA or a shRNA.

7. (canceled)

8. The engineered cytotoxic T cell of claim 1, wherein the engineered cytotoxic T cell is derived from an autologous donor or an allogeneic donor.

9. An engineered CD4+ helper T cell that
a. comprises a T cell receptor that binds to a target antigen and/or a nucleic acid encoding the T cell receptor; and
b. comprises a non-endogenous expression vector that includes a nucleic acid sequence encoding a CD8 alpha polypeptide comprising the amino acid sequence of SEQ ID NO: 36, SEQ ID NO: 37, SEQ ID NO: 38, SEQ ID NO: 47, SEQ ID NO: 48, or SEQ ID NO: 49, optionally wherein the nucleic acid sequence of the CD8 alpha polypeptide includes SEQ ID NO: 33, SEQ ID NO: 34 or SEQ ID NO: 35 and/or is operably linked to an expression control sequence.

10. The engineered CD4+ helper T cell of claim 9, wherein the TCR is a native TCR, a non-native TCR, or a mimic TCR or wherein the TCR is IG4, DMF5, or A6.

11. (canceled)

12. The engineered CD4+ helper T cell of claim 9, wherein the engineered cytotoxic T cell is derived from an autologous donor or an allogeneic donor.

13. The engineered cytotoxic T cell of claim 1, wherein the non-endogenous expression vector is a plasmid, a cosmid, a bacmid, a bacterial artificial chromosome (BAC), a yeast artificial chromosome (YAC), a viral vector, or a retroviral vector; or
wherein the expression control sequence is an inducible promoter, a constitutive promoter, a native promoter, or a heterologous promoter; or
wherein the target antigen comprises a tumor antigen.

14. The engineered CD4+ helper T cell of claim 9, wherein the expression control sequence is an inducible promoter, a constitutive promoter, a native promoter, or a heterologous promoter; or
wherein the non-endogenous expression vector is a plasmid, a cosmid, a bacmid, a bacterial artificial chromosome (BAC), a yeast artificial chromosome (YAC), a viral vector, or a retroviral vector; or
wherein the target antigen comprises a tumor antigen.

15. (canceled)

16. A composition comprising an effective amount of the engineered cytotoxic T cell of claim 1, and a pharmaceutically acceptable carrier.

17. A kit comprising
a. an expression vector that includes a nucleic acid sequence encoding a CD8 alpha amino acid sequence of SEQ ID NO: 36, SEQ ID NO: 37 SEQ ID NO: 38, SEQ ID NO: 47, SEQ ID NO: 48, or SEQ ID NO: 49, optionally wherein the nucleic acid sequence is any one of SEQ ID NO: 33, SEQ ID NO: 34, or SEQ ID NO: 35, and instructions for transducing CD4+ helper T cells with the expression vector; or
b. an expression vector that includes a nucleic acid sequence encoding a mutant CD8 alpha amino acid sequence of SEQ ID NO: 37, SEQ ID NO: 38, SEQ ID NO: 48 or SEQ ID NO: 49, optionally wherein the nucleic acid sequence is SEQ ID NO: 34 or SEQ ID NO: 35, and instructions for transducing cytotoxic T cells with the expression vector,
optionally wherein the kit further comprises a vector encoding an engineered T-cell receptor (TCR) that binds to a target antigen.

18. (canceled)

19. (canceled)

20. A method for treating cancer or inhibiting tumor growth in a subject in need thereof or for mitigating off-target reactivity/toxicity in a subject receiving adoptive T cell therapy comprising administering to the subject an effective amount of the engineered cytotoxic T cell of claim 1.

21. A method for treating cancer or inhibiting tumor growth in a subject in need thereof or mitigating off-target reactivity/toxicity in a subject receiving adoptive T cell therapy comprising administering to the subject an effective amount of the engineered CD4+ helper T cell of claim 9, optionally wherein the subject suffers from or is diagnosed with cancer.

22. (canceled)

23. The method of claim 20, wherein the cancer or tumor is selected from the group consisting of adrenal cancers, bladder cancers, blood cancers, bone cancers, brain cancers, breast cancers, carcinoma, cervical cancers, colon cancers, colorectal cancers, corpus uterine cancers, ear, nose and throat (ENT) cancers, endometrial cancers, esophageal cancers, gastrointestinal cancers, head and neck cancers, Hodgkin's disease, intestinal cancers, kidney cancers, larynx cancers, acute and chronic leukemias, liver cancers, lymph node cancers, lymphomas, lung cancers, melanomas, mesothelioma, myelomas, nasopharynx cancers, neuroblastomas, non-Hodgkin's lymphoma, oral cancers, ovarian cancers, pancreatic cancers, penile cancers, pharynx cancers, prostate cancers, rectal cancers, sarcoma, seminomas, skin cancers, stomach cancers, teratomas, testicular cancers, thyroid cancers, uterine cancers, vaginal cancers, vascular tumors, and metastases thereof.

24. The method of claim 20, wherein the engineered cytotoxic T cell is administered pleurally, intravenously, subcutaneously, intranodally, intratumorally, intrathecally, intrapleurally or intraperitoneally.

25. A method of preparing T cells for adoptive cell therapy comprising:
(A)
isolating cytotoxic T cells from a donor subject;
inactivating expression and/or activity of a wild-type CD8 alpha polypeptide comprising the amino acid sequence of SEQ ID NO: 36 or SEQ ID NO: 47 in the cytotoxic T cells;
transducing the cytotoxic T cells with a non-endogenous expression vector that includes a nucleic acid sequence encoding a mutant CD8 alpha polypeptide comprising the amino acid sequence of SEQ ID NO: 37, SEQ ID NO: 38, SEQ ID NO: 48, or SEQ ID NO: 49, optionally wherein the nucleic acid sequence of the mutant CD8 alpha polypeptide includes SEQ ID NO: 34 or SEQ ID NO: 35; and
administering the transduced cytotoxic T cells to a recipient subject; or
(B)
isolating CD4+ helper T cells from a donor subject;
transducing the CD4+ helper T cells with a non-endogenous expression vector that includes a nucleic acid sequence encoding a CD8 alpha polypeptide comprising the amino acid sequence of SEQ ID NO: 36, SEQ ID NO: 37, SEQ ID NO: 38, SEQ ID NO: 47, SEQ ID NO: 48, or SEQ ID NO: 49, optionally wherein the nucleic acid sequence of the CD8 alpha polypeptide includes SEQ ID NO: 33, SEQ ID NO: 34 or SEQ ID NO: 35; and
administering the transduced CD4+ helper T cells to a recipient subject.

26. (canceled)

27. The method of claim 25, wherein the donor subject and the recipient subject are the same or different.

28. The method of claim 25, wherein the T cells comprise a native T cell receptor (TCR), a non-native TCR, or a mimic TCR.
